(19) United States
(12) Patent Application Publication
(10) Pub. No.: US 2026/0357279 A1
(43) Pub. Date: Nov. 12, 2026

(54) METHOD OF PRODUCING POLYPHENOLS

(71) Applicant: NICOVENTURES TRADING LIMITED, London (GB)

(72) Inventors: Omer BAYAZEID, Winchester (GB); Anupriya THIND, London (GB); Sara BEN KHALED, Southampton (GB)

(21) Appl. No.: 19/479,063

(22) PCT Filed: Apr. 26, 2024
(86) PCT No.: PCT/GB2024/051093

§ 371 Date: Oct. 27, 2025

(30) Foreign Application Priority Data
Apr. 28, 2023 (GB) 2306351.4

(51) Int. Cl. C12N 15/82; C07D 311/30; C07H 17/07

(52) U.S. Cl. CPC C12N 15/8243; C07D 311/30; C07H 17/07

(57) ABSTRACT

The present disclosure relates to a composition, cells transformed with the composition, use of the composition, methods of using the composition, and products obtainable from the method.

### FIELD OF THE INVENTION

The present disclosure relates to a composition, cells transformed with the composition, use of the composition, methods of using the composition, and products obtainable from the method.

### BACKGROUND OF THE INVENTION

Polyphenols represent a diverse class of plant compounds that are thought to offer various health benefits. They are found naturally in plant foods and can act as antioxidants, thus protecting cells from damage by free radicals and reducing the risk of conditions such as cancer, diabetes and heart disease in those that consume them (Pham-Huy L A et al., Int J Biomed Sci. 2018, 4(2): 89-96). In addition, polyphenols are also thought to reduce inflammation and thus, may be beneficial in the management of many chronic illnesses.

Currently, more than 8000 phenolic structures have been identified. As a group, polyphenols are highly diverse and contain several sub-groups including phenolic acids, flavonoids, polyphenolic amides and other polyphenols (including resveratrol, lignans, curcumin, rosmarinic acid and ellagic acid). The largest sub-group is that of flavonoid compounds, which represent over 4000 of the 8000 currently identified polyphenol compounds.

Due to their many identified health benefits, polyphenols present a diverse class of bioactive compounds with potential nutritional and pharmaceutical uses (Williamson, Nutr Bull. 2017, 42(3): 226-235).

### SUMMARY OF THE INVENTION

In one aspect, there is provided a composition for promoting the expression of one or more polyphenols in a cell, wherein the composition comprises:
a) one or more first vector components encoding one or more transcription factors present in the flavonoid pathway; and optionally
b) one or more second vector components encoding one or more enzymes present in the flavonoid pathway.

In one embodiment, the one or more first vector components are provided in a first vector and the one or more second vector components are provided in a second vector.

In one embodiment, the one or more first vector components and the one or more second vector components are provided in a single vector.

In one aspect, there is provided a plant, plant part or plant cell transformed with the vector components according to any one of the composition embodiments described herein.

In one aspect, there is provided use of the composition according to any one of the composition embodiments described herein for promoting the expression of one or more polyphenols in algae, moss, or any plant, plant part, or plant cells.

In one aspect, there is provided a method of promoting the expression of one or more polyphenols in a cell, comprising the step of:
a) introducing the composition according to any one of the composition embodiments described herein into a cell.

Optionally, the method further comprises the steps of:
b) extracting the one or more polyphenols from the cell to produce an extract;
c) detecting the one or more polyphenols in the extract; and
d) purifying the one or more polyphenols from the extract.

In one aspect, there is provided a polyphenol obtainable from the method according to any one of the method embodiments described herein.

### BRIEF DESCRIPTION OF THE DRAWINGS

Embodiments of the invention will now be described, by way of example only, with reference to accompanying drawings, in which:

FIGS. 1A-C show Nicotiana tabacum leaves on day 3 and day 7 following transfection with a composition comprising flavonoid pathway transcription factors according to the invention disclosed herein.

FIGS. 2A and 2B show HPTLC results for set 3 and set 9 of FIGS. 1A-C against a Quercetin standard (2A) and Kaempferol standard (2B). FIG. 2A also shows the results for set 10 (control). Stationary Phase; Merck, HPTLC Silica gel 60 F₂₅₄, Mobile phase; Chloroform:Methanol 9:1 (v/v), Chloroform:Methanol:Water 80:20:2 (v/v/v), Chloroform:Methanol:Water 61:32:5 (v/v/v), Toluene:Ethyl acetate:Acetic acid:Chloroform 5:5:1:2 (v/v/v/v). All samples dissolved in Methanol.

FIG. 3 schematically shows a customized pCAmbia2300 vector encoding AmDEL and AmROS1 for plant cell lines and BY2 cells.

FIG. 4 schematically shows a customized pCAmbia2300 vector encoding AtMYB12 for plant cell lines and BY2 cells.

FIG. 5 schematically shows a customized pCAmbia2300 vector encoding AmDEL, AmROS1 and AtMYB12 for plant cell lines and BY2 cells FIG. 6 schematically shows a customized pMBL5DL108 vector encoding AmDEL and AmROS1 for moss.

FIG. 7 schematically shows a customized pUC-GW-Amp-Cat174-nohygr vector encoding AtMYB12 for algae.

FIG. 8 schematically shows a customized pUC-GW-Amp vector encoding AmDEL and AmROS1 for algae.

FIG. 9 schematically shows a customized pCAmbia2300 vector encoding NtAN2 for BY2 cells and Nicotiana species.

FIG. 10 schematically shows a customized pCAmbia2300 vector encoding AtTT8 for BY2 cells and Nicotiana species.

FIGS. 11A-Q show the synthesis pathways of various polyphenols, where transcription factors and/or enzymes present in the flavonoid pathway may modulate expression. The figures do not provide an exhaustive list of the polyphenols that may be derived. The skilled person will understand that further polyphenols may be expressed with additional or alternative enzyme or transcription factor combinations. FIGS. 11A and 11B show the phenylpropanoid pathway, which is the first stage of polyphenol synthesis in the flavonoid pathway. FIG. 11C follows on from FIG. 11B showing the downstream pathway of pinocembrin chalcone. FIGS. 11D and 11E show two of the downstream pathways of p-coumaroyl-CoA of FIG. 11A. FIG. 11D shows the conversion of p-coumaroyl-CoA to isoliquiritigenin and the subsequent products, whilst 11E shows the conversion of p-coumaroyl-CoA to naringenin chalcone and the pathway leading to eriodictyol (1), apigenin (2), dihydrokaempferol (3), and dihydroquercetin (4). FIGS. 11F and 11G show downstream pathways of (1) from FIG. 11E. FIG. 11H shows a downstream pathway of (2) from FIG. 11E, whilst FIG. 11I shows a downstream pathway that both (1) and (2) from FIG. 11E can feed into. FIGS. 11J and 11K show downstream pathways of (3) from FIG. 1E. FIG. 11L shows a downstream pathway of (4) from FIG. 1E. FIGS. 11M-O show downstream pathways for (3) and (4) from FIG. 11E and Dihydromyricetin from FIG. 11K, whilst FIGS. 11P and 11Q show alternative downstream pathways for the same molecules. For all figures, the boxes (both solid and dashed lines) denote the transcription factors that may be present. Enzymes are denoted on the relevant arrows that indicate the various conversion steps of the molecules. Enzymes: Phenylalanine ammonia-lyase (PAL), cinnamate 4-hydroxylase (C4H), caffeic acid O-methyltransferase (COMT), ferulate-5 hydroxylase (F5H), 4-coumarate:CoA ligase (4CL), cinnamate-CoA ligase (CNL), hydroxycinnamoyltransferase (HCT), p-coumaroyl shikimate 3′-hydroxylase (C3′H), caffeoyl shikimate esterase (CSE), caffeoyl-CoA O-methyltransferase (CCoAOMT), chalcone synthase (CHS), chalcone isomerase (CHI), flavone synthase II (FNS II), O-methyltransferase (OMT), Flavanone 3-hydroxylase/Flavanone 3p-hydroxylase (F3H), NADPH, c-UDP-glycosyltransferase (C-UGT), O-glucuronosyltransferase (OGT), .UDP-glycosyltransferase (UGT), chalcone reductase (CHR), flavanone 2-hydroxylase (F2H), flavonoid 3′-hydroxylase (F3′H), isoflavones synthase (IFS), 2-hydroxyisoflavanone dehydratase (HID), flavone synthase (FNS), UTP-glucose-1-phosphate (UGP-sugar type*), c-UTP-glucose-1-phosphate (C-UGP-sugar type*), flavonol synthase (FLS), flavonoid 3′,5′-hydroxylase (F3′,5′H), dihydroflavonol 4-reductase (DFR), anthocyanidin synthase (ANS), 0-malonyltransferase (OMNT), leucoanthocyanidin reductase (LAR). Transcription factors (TFs): Del, Ros, MYB12, AN2, TT8, NtAn1b, NtAn11-1, NtMYB330, AtPAP1, AtTT2, MtMYB14, VvMYBPA1, CsMYB5b, CsMYB75, CsMYB5b. * sugar type may be glucose, rutinose, xylose, galactose, rhamnose.

FIG. 12 shows HPTLC results for insertion of eight different combinations of genes and transcription factors inserted into the leaves of Nicotiana tabacum or Nicotiana benthamiana, against (+)Catechin, (−)Epicatechin and (−)Epigallocatechin standards. Stationary phase: Merck, HPTLC Silica gel 60 F₂₅₄. Mobile phase: Cyclopentyl Methyl Ether, Tetrahydrofuran, Water, Formic acid 40:24:1:1 (VN). Derivatization reagent: 10% H₂SO₄ in Methanol. All samples dissolved in 100% Methanol. NT=Nicotiana tabacum, NB=Nicotiana benthamiana. Gene and transcription factor combinations were as follows: Set 1: anthocyanidin reductase (ANR) & leucoanthocyanidin reductase (LAR); Set 2: ANR, LAR, DEL, & ROS; Set 3: ANR, LAR, DEL, ROS, & MYB330; Set 4: ANR, LAR, DEL, ROS, & MYB12; Set 5: ANR, LAR, DEL, ROS, & MYB5a; Set 6: ANR, LAR, DEL, ROS, &AN2; Set 7: ANR, LAR, DEL, ROS1, MYB330, MYB12, MYB5a, &AN2; Set 8: GFP.

FIG. 13 shows HPTLC results for three different combinations of genes and transcription factors inserted into the leaves of Nicotiana tabacum or Nicotiana benthamiana, against an Apigenin-7-O-glucoside standard. Stationary phase: Merck, HPTLC Silica gel 60 F₂₅₄. Mobile phase: Ethanol, Dichloromethne, Water, Formic acid 16:16:4:1 (VN). Derivatization reagent: 10% H₂SO₄ in Methanol. All samples dissolved in 100% Methanol. NT=Nicotiana tabacum, NB=Nicotiana benthamiana. Gene and transcription factor combinations were as follows: Set 1: AtMYB12, soluble flavone synthase (FNSI), UGT84A23, & UGT84A24; Set 2: AtMYB12, FNSI, UGT84A23, UGT84A24, DEL, & ROS1; Set 3: GFP.

FIGS. 14A and 14B show HPTLC results for eight different combinations of genes and transcription factors inserted into the leaves of Nicotiana tabacum (14A) or Nicotiana benthamiana (14B), against green tea, (+)catechin, (−)catechin, (−)epicatechin, (−)epigallocatechin and pelargonidin standards. Stationary phase: Merck, HPTLC Silica gel 60 F₂₅₄. Mobile phase: Cyclopentyl Methyl Ether, Tetrahydrofuran, Water, Formic acid 40:24:1:1 (VN). Derivatization reagent: 10% H₂SO₄ in Methanol. All samples dissolved in 100% Methanol. Gene and transcription factor combinations were as follows: Set 1: ANR, LAR, DEL, & ROS; Set 2: MYB330; Set 3: MYB330 & MYB12; Set 4: MYB330, MYB12, & MYB5a; Set 5: MYB330, MYB12, MYB5a, & AN2; Set 6: ANR, LAR, MYB330, MYB12, MYB5a, & AN2; Set 7: DEL, ROS, MYB330, MYB12, MYB5a, & AN2; Set 8: ANR, LAR, DEL, ROS1, MYB330, MYB12, MYB5a, & AN2.

### BRIEF DESCRIPTION OF THE SEQUENCES

A summary of sequence identifiers used throughout the subject specification and the corresponding sequence listing is provided wherein:

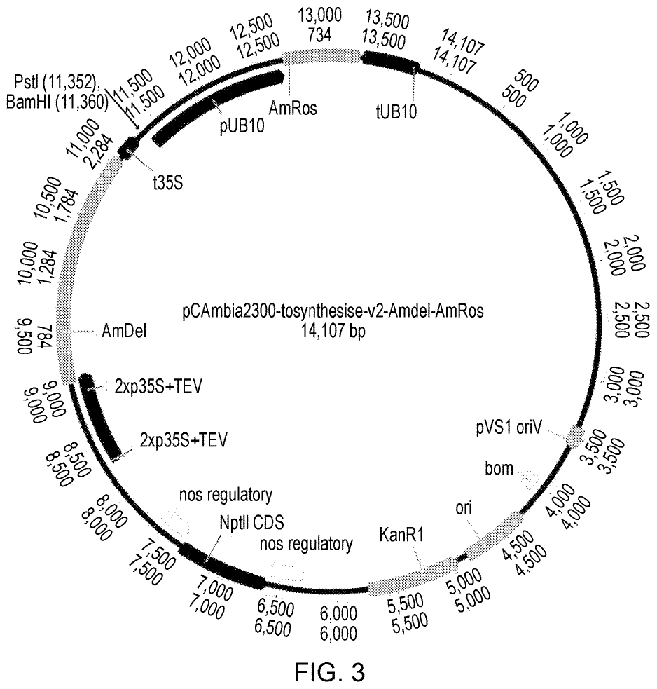

SEQ ID NO. 1 corresponds to the nucleic acid sequence of the pCAmbia2300 vector of FIG. 3, which encodes AmDEL and AmROS1.

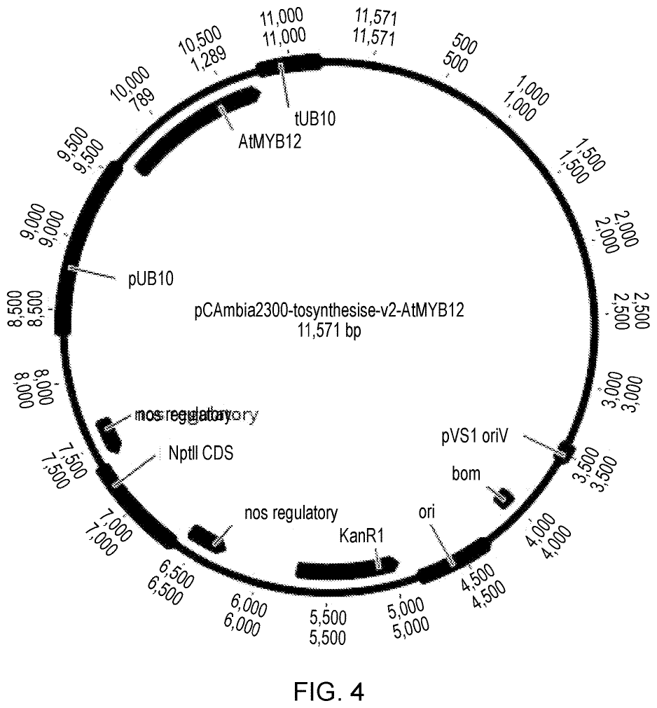

SEQ ID NO. 2 corresponds to the nucleic acid sequence of the pCAmbia2300 vector of FIG. 4, which encodes AtMYB12.

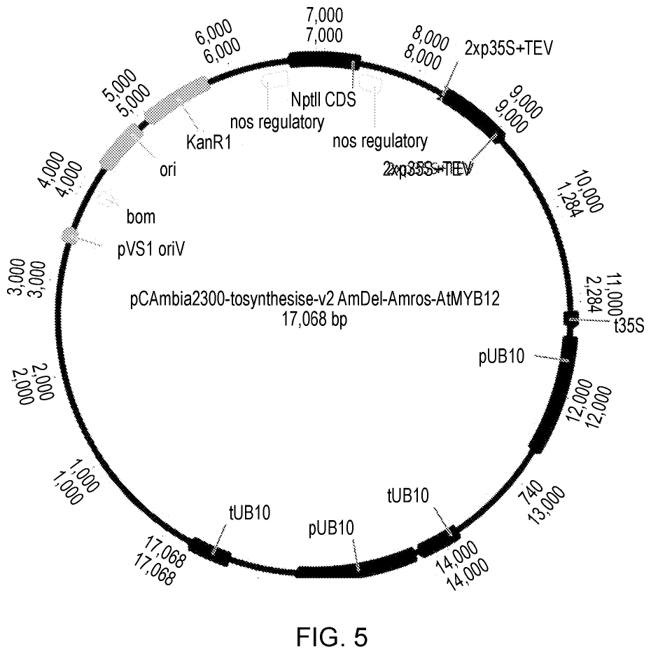

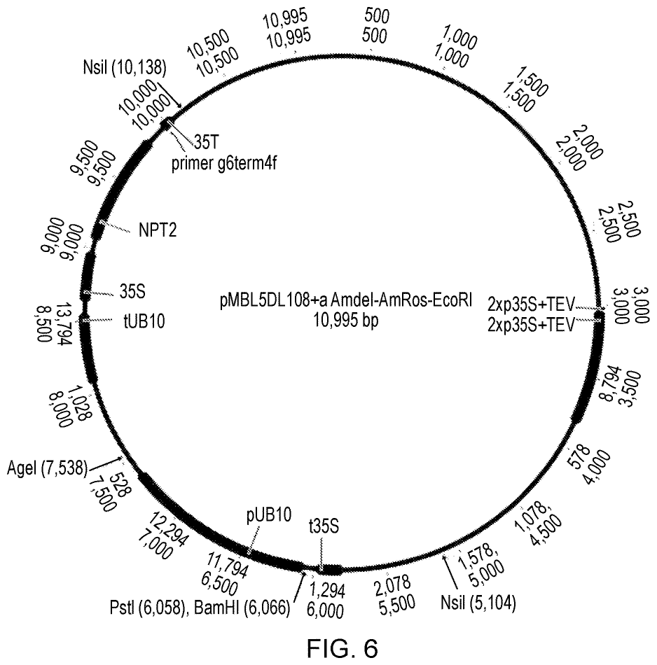

SEQ ID NO. 3 corresponds to the nucleic acid sequence of the pCAmbia2300 vector of FIG. 5, which encodes AmDEL, AmROS1 and AtMYB12.

SEQ ID NO. 4 corresponds to the nucleic acid sequence of the pMBL5DL108 vector of FIG. 6, which encodes AmDEL and AmROS1.

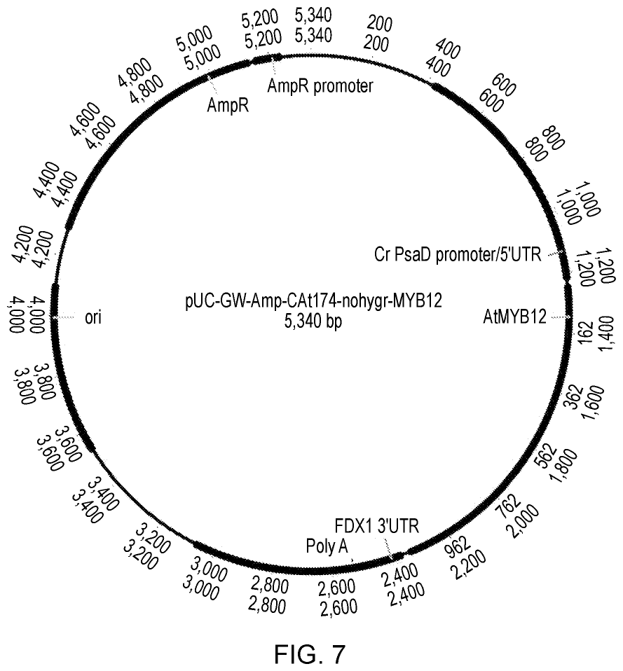

SEQ ID NO. 5 corresponds to the nucleic acid sequence of the pUC-GW-Amp-Cat174-nohygr vector of FIG. 7, which encodes AtMYB12.

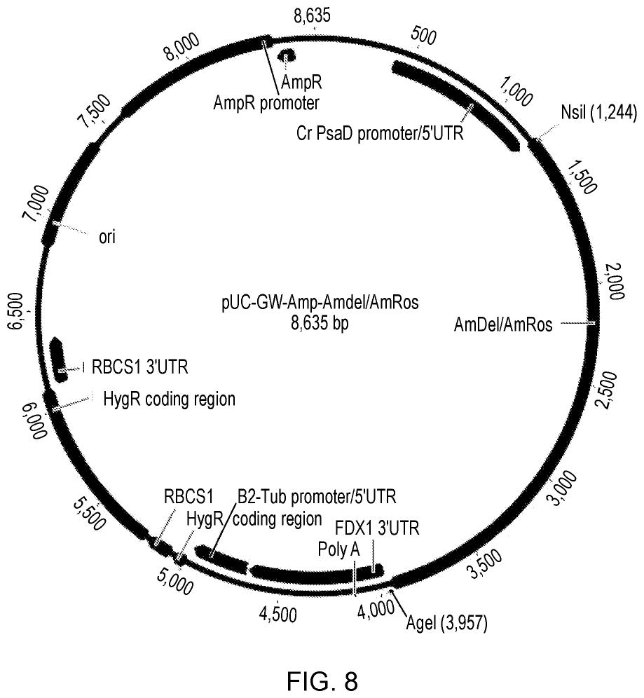

SEQ ID NO. 6 corresponds to the nucleic acid sequence of the pUC-GW-Amp vector of FIG. 8, which encodes AmDEL and AmROS1.

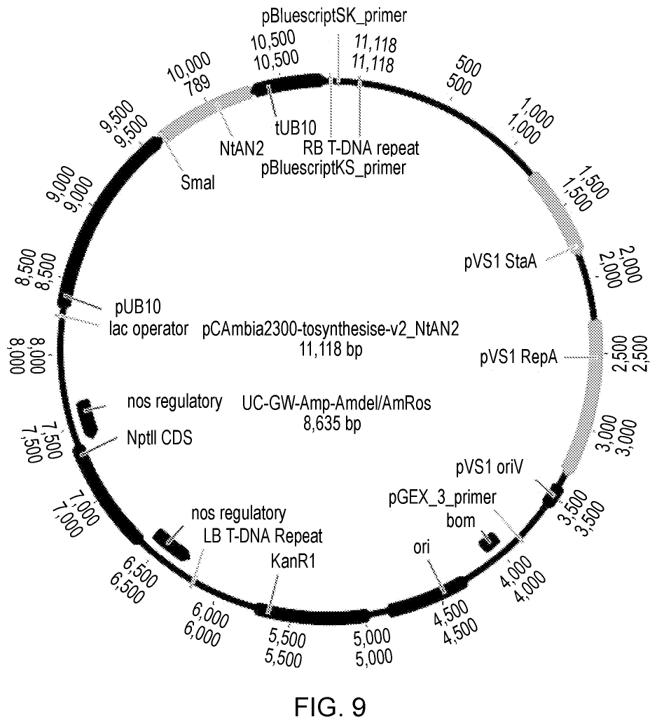

SEQ ID NO. 7 corresponds to the nucleic acid sequence of the pCAmbia2300 vector of FIG. 9, which encodes AtAN2.

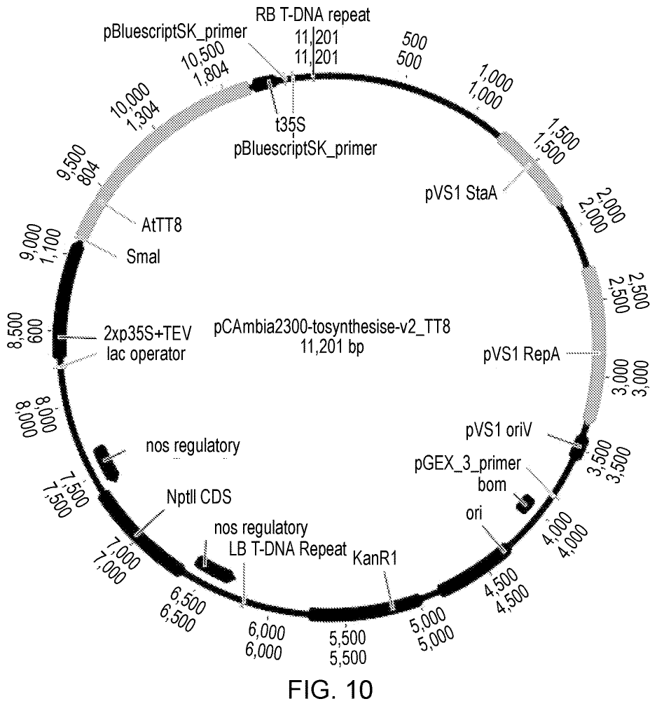

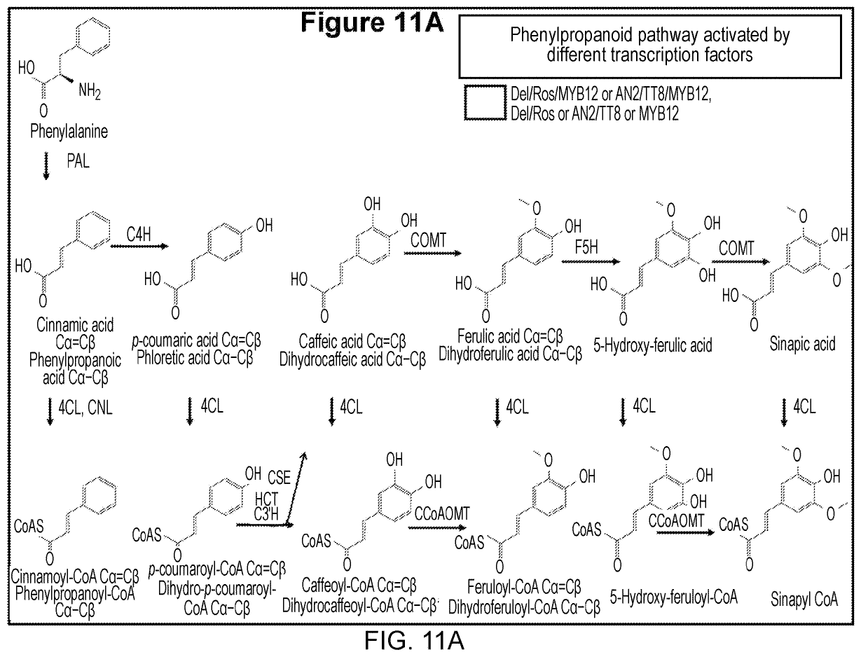

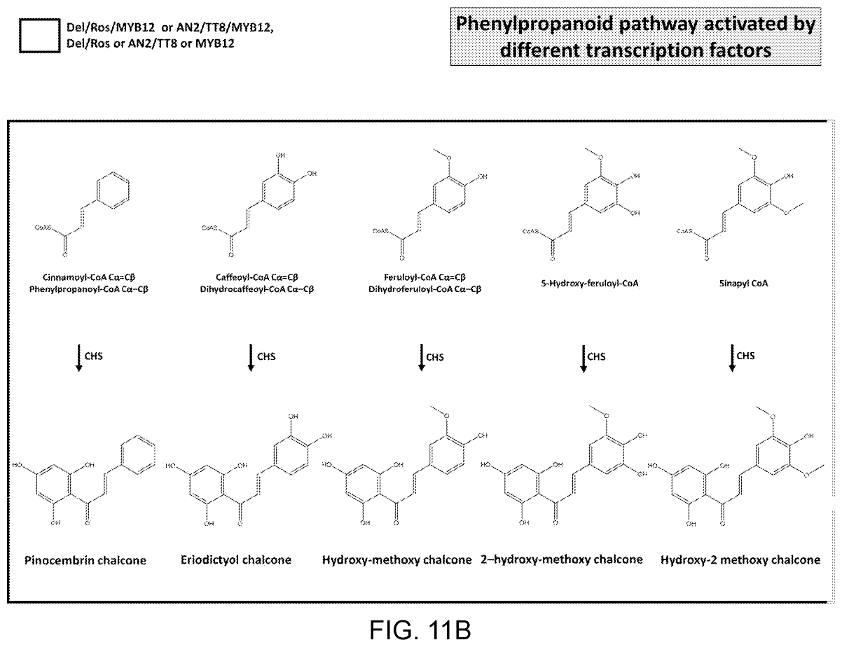

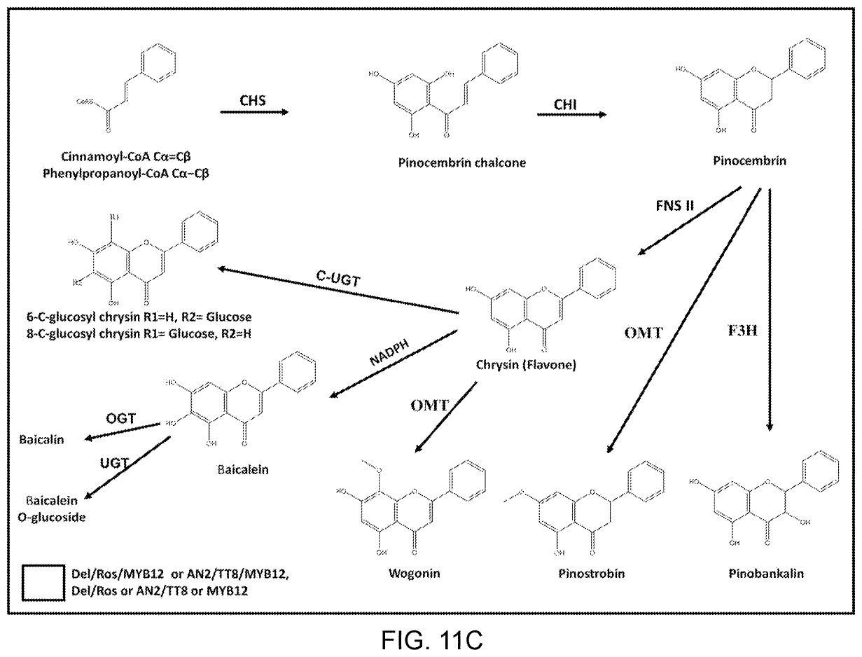

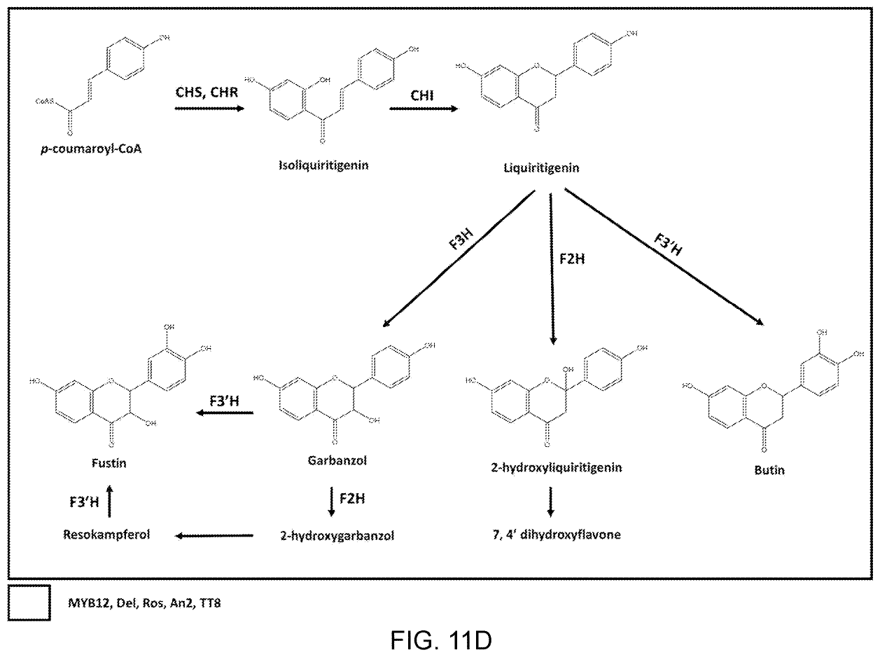

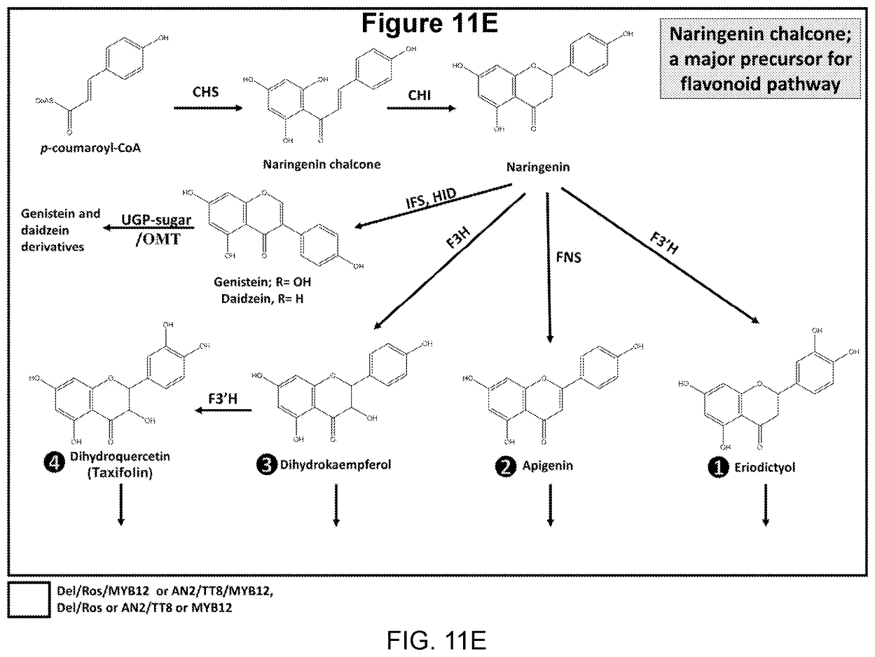

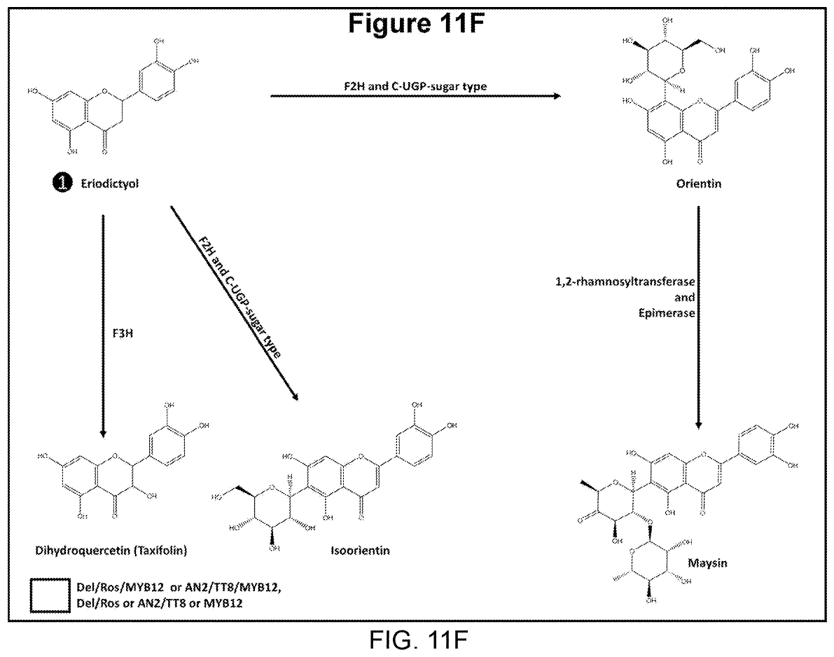

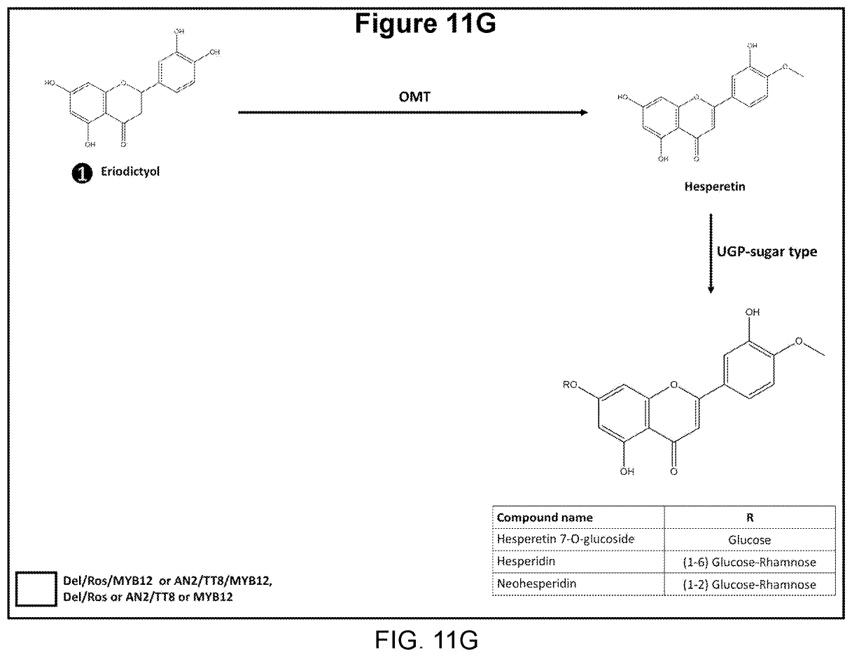

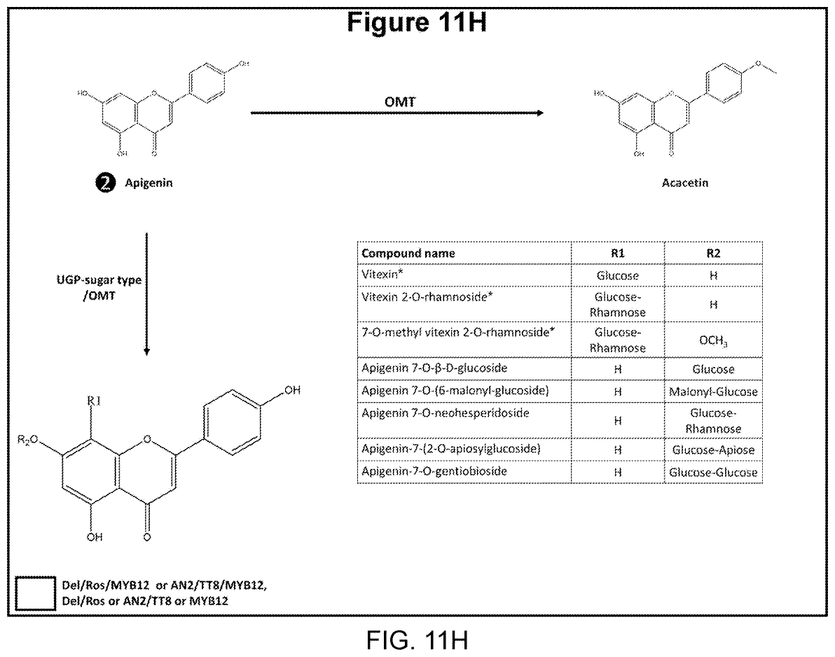

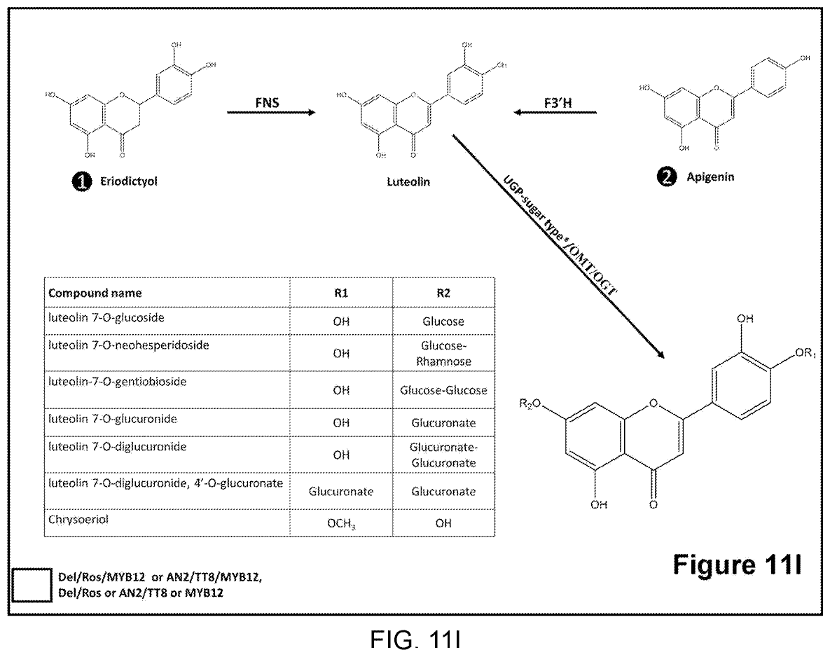

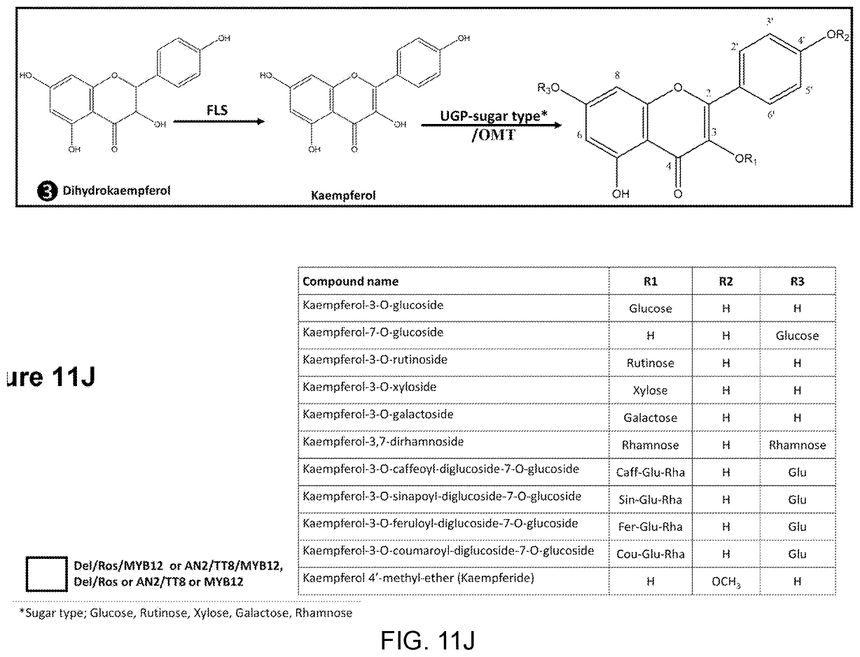

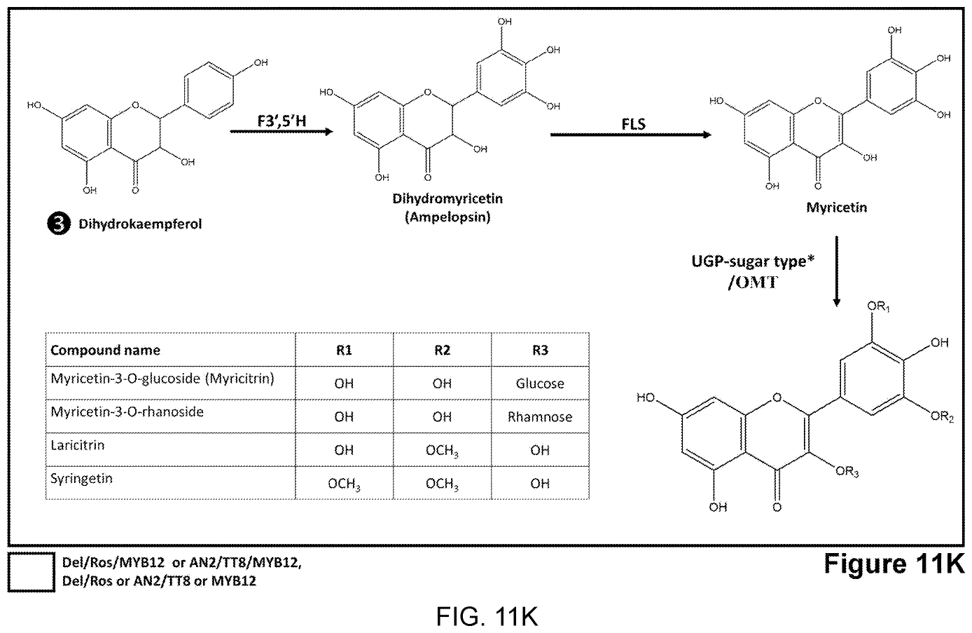

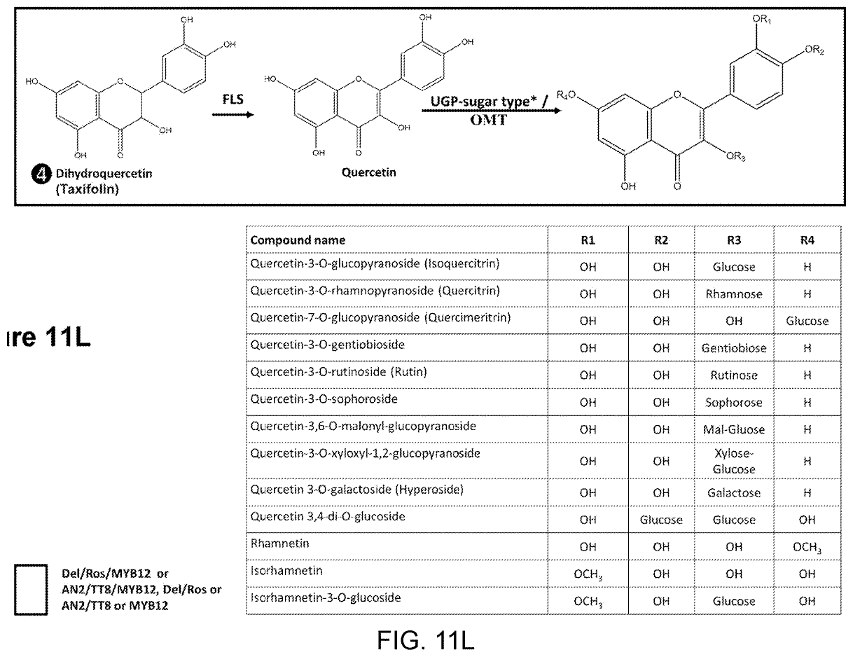

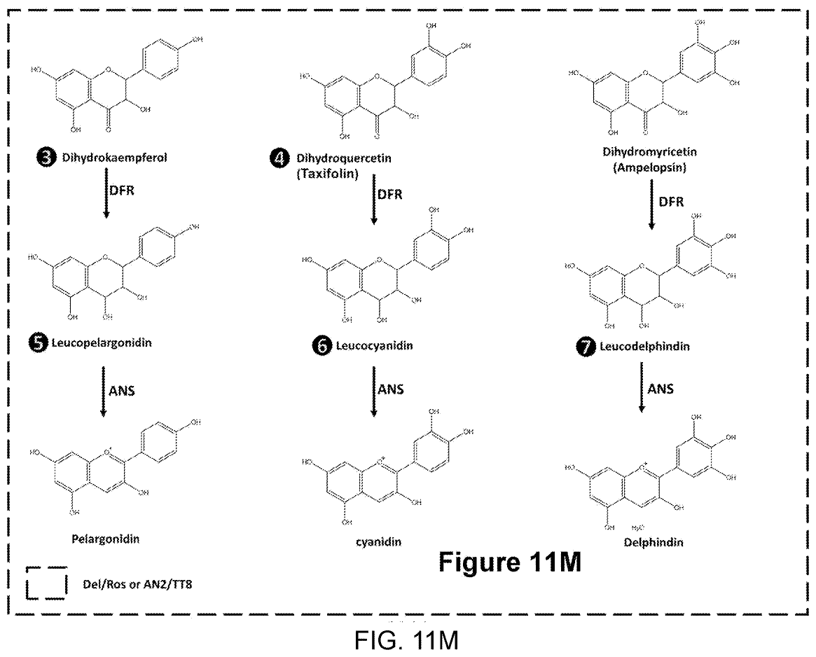

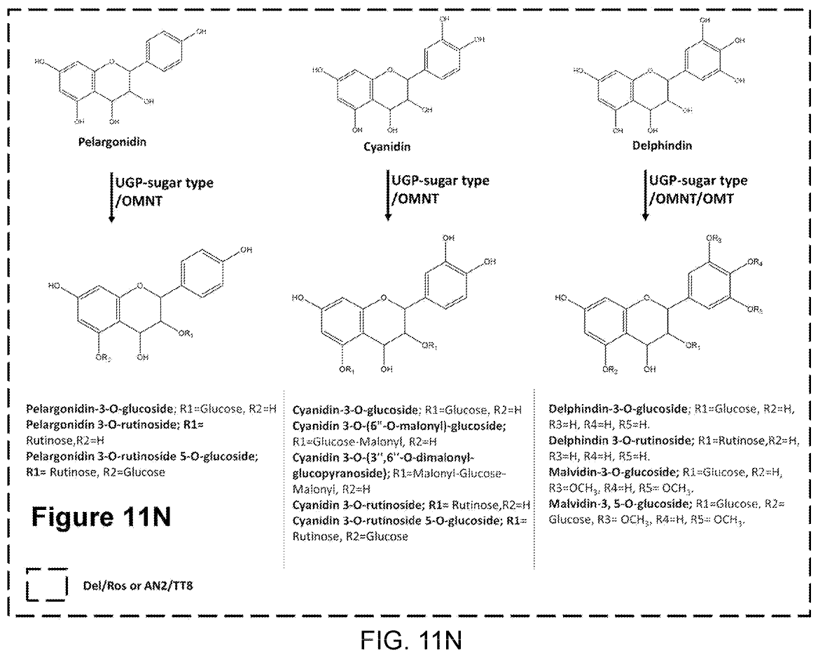

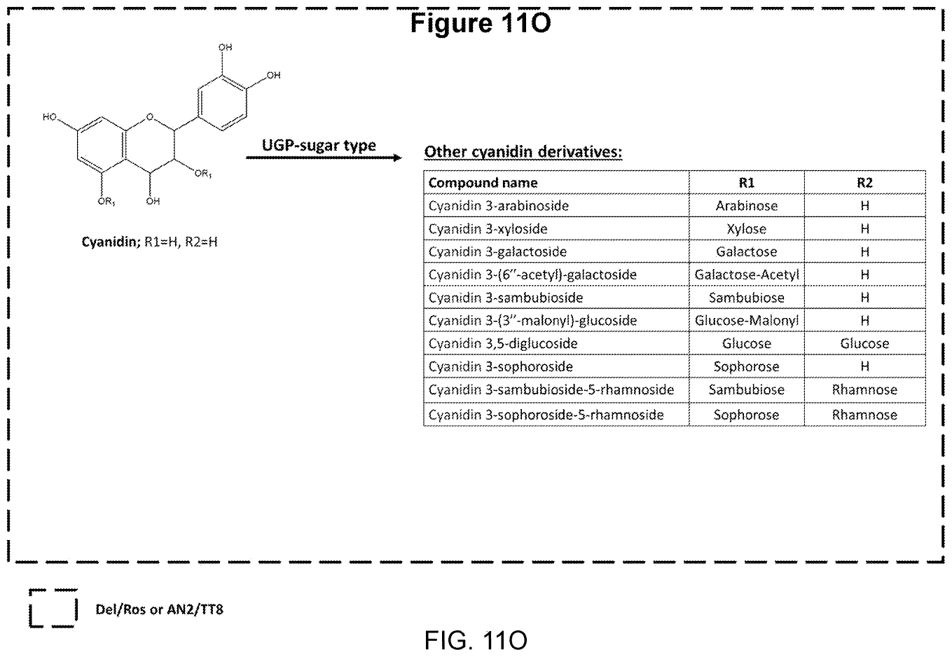

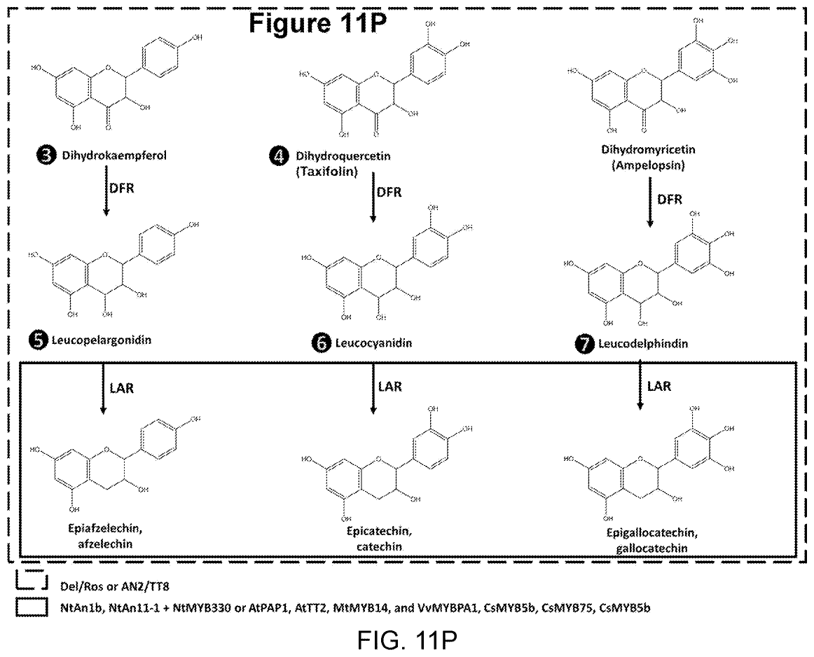

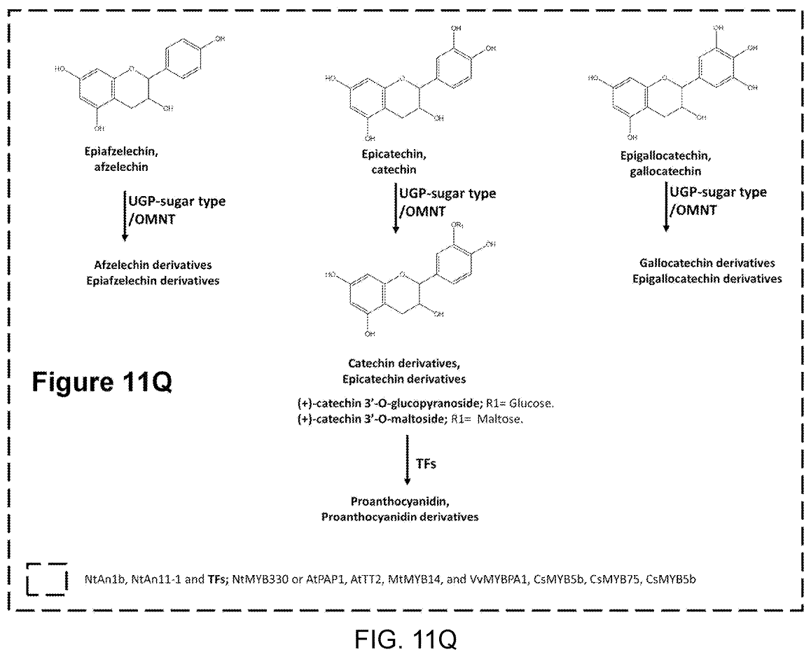

SEQ ID NO. 8 corresponds to the nucleic acid sequence of the pCAmbia2300 vector of FIG. 10, which encodes AtTT8.

### DEFINITIONS

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. Accordingly, the terms defined immediately below are more fully defined by reference to the specification as a whole.

It is to be understood that the term “polyphenol” is interchangeable with the term “polyphenolic compound”. “Polyphenols” and/or “polyphenolic compounds” are a class of naturally occurring organic compounds characterised by multiples of phenol units.

“Flavonoids” are a class of polyphenol secondary metabolites. Therefore, the skilled person would understand that the term “polyphenols” encompasses “flavonoids”. Therefore, the term “polyphenol(s)” may be replaced by the term “flavonoid(s)” herein.

Where “transcription factors present in the flavonoid pathway” or “enzymes present in the flavonoid pathway” are disclosed, the skilled person will understand that the flavonoid pathway may encompass all of the pathway from the phenylalanine starting point through to a flavonoid end product. The flavonoid pathway may be divided into three subdivisions. The first stage being the phenylpropanoid synthesis pathway, the second stage being early flavonoid biosynthesis, and the third stage being late flavonoid biosynthesis.

It is to be understood that a polypeptide (synonymous with “protein” and/or “enzyme”) may be coded for by more than one nucleotide sequence due to the degeneracy of the genetic code. Therefore, a polynucleotide encoding the first and/or second vector components of the invention may have at least 70%, 75%, 80%, 85%, 90%, 95%, 96%, 97%, 98% or 99% sequence identity to the disclosed nucleotide sequences (SEQ ID NO: 1, 2, 3, 4, 5, 6, 7, or 8).

Other definitions of terms may appear throughout the specification. Before the exemplary aspects and embodiments are described in more detail, it is to understand that this disclosure is not limited to particular aspects or embodiments described, as such may, of course, vary. It is also to be understood that the terminology used herein is for the purpose of describing particular aspects or embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an enzyme”, “a polyphenol” or “a flavonoid” includes a plurality of such candidate agents and equivalents thereof known to those skilled in the art, and so forth.

### DETAILED DESCRIPTION OF THE INVENTION

Polyphenols present a diverse family of compounds found in plant-based foods and beverages that elicit a number of beneficial effects on human health. Polyphenols can be divided into different groups according to the phenol rings such as, phenolic acids, flavonoids, stilbenes and lignans. They mostly occur in conjugated forms, with one or more sugar molecules that bind to hydroxyl groups of the aromatic ring. However, in some cases the sugar may bind directly to the aromatic carbon instead. Other linkages to compounds such as amines, carboxylic and organic acids, lipids or other phenols may also occur.

Polyphenols have shown numerous biological activities and health benefits for prevention and treatment of age-related diseases and they are very popular for their antioxidant and anti-inflammatory effects, which may be beneficial in the prevention and treatment of neurodegenerative diseases (Li et al., Nutrients. 2014, 6(12): 6020-6047). Due to this, and the potential for polyphenols to be isolated and added to food, beverages and pharmaceuticals, such as supplements, it would be advantageous to develop methods to promote expression of polyphenols of interest, such that they can be extracted for further use.

In one aspect, there is provided a composition for promoting the expression of one or more polyphenols in a cell, wherein the composition comprises:
a) one or more first vector components encoding one or more transcription factors present in the flavonoid pathway; and optionally
b) one or more second vector components encoding one or more enzymes present in the flavonoid pathway.

In one embodiment, the one or more first vector components are provided in a first vector and optionally the one or more second vector components are provided in a second vector.

In one embodiment, the one or more first vector components and optionally the one or more second vector components are provided in a single vector.

The skilled person will understand that the number of vectors which comprise the vector components of the composition, will depend on the transcription factors and optionally the enzymes present in the flavonoid pathway that they wish to introduce to a cell. The claimed composition provides flexibility to tailor the encoded vector components to the suit the desired promotion of polyphenols.

It will be understood that in some embodiments, the one or more first vector components may be present in a single vector. In other embodiments, the one or more first vector components may be present in more than one vector, such as two, three, four, five, six or more vectors.

It will be understood that in some embodiments, the one or more second vector components may be present in a single vector. In other embodiments the one or more second vector components may be present in more than one vector, such as two, three, four, five, six or more vectors.

The number of vectors used for each vector component is independently selected such that the one or more first vector components may be present in a single vector whilst the one or more second vector components may be present in more than one vector, and vice versa.

It will also be understood that the invention contemplates all combinations of the vector components disclosed herein. For example, the invention contemplates a first vector comprising one or more first vector components, a second vector comprising one or more second vector components and a third vector comprising one or more first vector components and one or more second vector components.

In one embodiment, the total nucleotide sequence length encoded by the first and/or second vector components is 200 KB or less.

In one embodiment, the total nucleotide sequence length encoded by the first and/or second vector components is 190 KB or less.

In one embodiment, the total nucleotide sequence length encoded by the first and/or second vector components is 180 KB or less.

In one embodiment, the total nucleotide sequence length encoded by the first and/or second vector components is 170 KB or less.

In one embodiment, the total nucleotide sequence length encoded by the first and/or second vector components is 160 KB or less.

In one embodiment, the total nucleotide sequence length encoded by the first and/or second vector components is 150 KB or less.

In one embodiment, the total nucleotide sequence length encoded by the first and/or second vector components is 140 KB, 130 KB, 120 KB, 110 KB, 100 KB, 90 KB, 80 KB, 70 KB, 60 KB, 50 KB, 40 KB, 30 KB, 20 KB, 10 KB or less.

In one embodiment, the total nucleotide sequence length encoded by the first and/or second vector components is 10 KB or more.

In one embodiment, the total nucleotide sequence length encoded by the first and/or second vector components is 20 KB or more.

In one embodiment, the total nucleotide sequence length encoded by the first and/or second vector components is 30 KB or more.

In one embodiment, the total nucleotide sequence length encoded by the first and/or second vector components is 40 KB or more.

In one embodiment, the total nucleotide sequence length encoded by the first and/or second vector components is 50 KB or more.

In one embodiment, the total nucleotide sequence length encoded by the first and/or second vector components is 60 KB, 70 KB, 80 KB, 90 KB, 100 KB, 110 KB, 120 KB, 130 KB, 140 KB, 150 KB, 160 KB, 170 KB, 180 KB, 190 KB, 200 KB or more.

In one embodiment, the total nucleotide sequence length encoded by the first and/or second vector components is between 100 KB and 150 KB.

The total nucleotide sequence length of the first and/or second vector components will depend on whether they are to be inserted independently into two or more vectors, or in combination in a single vector. The skilled person will understand that the total nucleotide sequence length of the vector components is dependent on the vector construct to which they are to be inserted and thus could be greater than 200 KB in certain circumstances.

The skilled person will also understand that the vector construct in which the first vector component and/or second vector component are contained will be selected based on the skilled persons knowledge of suitable vectors for the species of interest. The vector constructs disclosed herein are examples of suitable constructs for algae, moss and plant species. However, other vector constructs may also be appropriate and the skilled person would be aware how to select an appropriate vector construct for their intended species.

Therefore, the skilled person will use their knowledge and the information disclosed herein to determine the total sequence length and the vector construct required for efficient transformation into the species of interest.

In one embodiment, one or more vectors comprising the first vector component and/or second vector component are added to a cell.

In one embodiment, two or more vectors comprising the first vector component and/or second vector component are added to a cell.

In one embodiment, three or more vectors comprising the first vector component and/or second vector component are added to a cell.

In one embodiment, four or more vectors comprising the first vector component and/or second vector component are added to a cell.

In one embodiment, five or more vectors comprising the first vector component and/or second vector component are added to a cell.

In one embodiment, six or more vectors comprising the fi first vector component and/or second vector component are added to a cell.

In one embodiment, seven or more vectors comprising the first vector component and/or second vector component are added to a cell.

In one embodiment, eight or more vectors comprising the first vector component and/or second vector component are added to a cell.

In one embodiment, nine or more vectors comprising the first vector component and/or second vector component are added to a cell.

In one embodiment, ten or more vectors comprising the first vector component and/or second vector component are added to a cell.

In one embodiment, up to ten vectors comprising the first vector component and/or second vector component are added to a cell.

In one embodiment, the cell is a plant cell.

In a further embodiment, the plant cell is an algae cell, moss cell, or any cell or cell line derived from the genus Nicotiana.

In yet a further embodiment, the cell or cell line derived from the genus Nicotiana is selected from the group comprising or consisting of Nicotiana tabacum, Nicotiana benthamiana, or BY-2 cells.

In an alternative embodiment, the cell is a yeast cell or a bacterial cell.

The skilled person will understand that the type of vector selected will depend on its intended purpose. The vector may be any suitable type of vector such as a plasmid, cosmid, phage, or viral vector.

In one embodiment, the vector comprising the first vector component and/or second vector component is a plasmid vector.

In a further embodiment, the plasmid vector is a plasmid binary vector.

We have found that either transcription factors alone, or a combination of enzymes and transcription factors leads to promotion of the expression of desired polyphenol compounds.

We have also discovered that particular combinations of transcription factors, disclosed herein, drive the expression of polyphenols from unexpected branches of the synthesis pathway.

For ease of reference, these and further aspects of the present invention are now discussed under appropriate section headings. However, the teachings under each section are not necessarily limited to each particular section.

Transcription factors present in the flavonoid pathway Flavonoids are plant secondary polyphenol metabolites, which can be divided into six major subgroups according to their molecular structures. They are endogenously expressed in higher plants, however, the early biosynthetic steps of the pathway have also been found in the bryophytes Physcomitrella patens and Marchantia polymorpha, suggesting that flavonoids may have originally evolved as chemical messengers. Flavonoid biosynthesis is principally modulated at the transcriptional level, thus there are a variety of transcription factors associated with the various biosynthetic steps (Li, Plant Signal Behav. 2014, 9: e27522).

As described herein, the composition for promoting the expression of one or more polyphenols comprises one or more first vector components encoding one or more transcription factors present in the flavonoid pathway.

In one embodiment, the one or more transcription factors present in the flavonoid pathway are selected from the group comprising or consisting of myeloblastosis (MYB) type transcription factors and/or basic helix-loop-helix (bHLH) type transcription factors.

In a further embodiment, the one or more transcription factors present in the flavonoid pathway are independently derived from a plant species.

The skilled person will understand that the transcription factors listed above and below may be derived from different plant species. Therefore, the listed transcription factors are intended to encompass those derived from any species including, but not necessarily limited to the prefixes At, Vv, Ib, Gh, Gm, Md, Gt, Ms, Co, Ej, Mg, Pc, Ppy, Pb, Par, Pi, Pdm, Pav, Pcr, Pcf, Pd, Ppr, Ps, Fa, Fv, Rh, Ri, Hv, Bo, Mr, Eg, Es, In, Dk, Mt, Lh, Lj, Zm, Pp, Cs, Og, Py, Pt, Ptr, Pto, St, Ta, Nt, and Le.

The skilled person will also understand that different plant species have their own transcription factors present in the flavonoid pathway. Therefore, the lists of transcription factors present in the flavonoid pathway provided above are non-limiting and the skilled person would select the most appropriate transcription factors based on the plant species of interest.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the one or more first vector components encode two or more transcription factors present in the flavonoid pathway.

In one embodiment, the two or more transcription factors present in the flavonoid pathway are selected from the group comprising or consisting of myeloblastosis (MYB) type transcription factors and/or basic helix-loop-helix (bHLH) type transcription factors.

In a further embodiment, the two or more transcription factors present in the flavonoid pathway are independently derived from a plant species.

In one embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the one or more vector components encode three or more transcription factors present in the flavonoid pathway.

In one embodiment, the three or more transcription factors present in the flavonoid pathway are selected from the group comprising or consisting of myeloblastosis (MYB) type transcription factors and/or basic helix-loop-helix (bHLH) type transcription factors.

In a further embodiment, the three or more transcription factors present in the flavonoid pathway are independently derived from a plant species.

In one embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes four or more transcription factors present in the flavonoid pathway.

In one embodiment, the four or more transcription factors present in the flavonoid pathway are selected from the group comprising or consisting of myeloblastosis (MYB) type transcription factors and/or basic helix-loop-helix (bHLH) type transcription factors.

In a further embodiment, the four or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes five or more transcription factors present in the flavonoid pathway.

In one embodiment, the five or more transcription factors present in the flavonoid pathway are selected from the group comprising or consisting of myeloblastosis (MYB) type transcription factors and/or basic helix-loop-helix (bHLH) type transcription factors.

In a further embodiment, the five or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes six or more transcription factors present in the flavonoid pathway.

In one embodiment, the six or more transcription factors present in the flavonoid pathway are selected from the group comprising or consisting of myeloblastosis (MYB) type transcription factors and/or basic helix-loop-helix (bHLH) type transcription factors.

In a further embodiment, the six or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes 7, 8, 9, 10, 11, 12, 13, 14, 15, 16, 17, 18, 19, 20, 21, 22, 23, 24, or 25 or more transcription factors present in the flavonoid pathway.

In one embodiment, the 7, 8, 9, 10, 11, 12, 13, 14, 15, 16, 17, 18, 19, 20, 21, 22, 23, 24, or 25 or more transcription factors present in the flavonoid pathway are selected from the group comprising or consisting of myeloblastosis (MYB) type transcription factors and/or basic helix-loop-helix (bHLH) type transcription factors.

In a further embodiment, the 7, 8, 9, 10, 11, 12, 13, 14, 15, 16, 17, 18, 19, 20, 21, 22, 23, 24, or 25 or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

The skilled person will be aware that the maximum number of transcription factors encoded by the second vector component will be dependent on the constraints of the vector construct chosen. E.g. the total sequence length that the vector construct can support.

In embodiments where each transcription factor may be independently derived from a plant species, it should be noted that each transcription factor need not necessarily be derived from the same plant species.

MYB proteins are transcription factors found in all eukaryotes. However, whilst only a few unique MYBs are encoded in unikonts (the phylogenetic group comprising animals and fungi), there are up to ~200 unique MYB genes found in the plant lineage. Of the MYB proteins present in plants, R2R3 MYB transcription factors are the most widely encoded, with many plant species encoding >100 unique R2R3 MYB.

In one embodiment, the MYB type transcription factor is a member of the R2R3 MYB transcription factor family.

In one embodiment, the MYB type transcription factor is one or more of MYB0, MYB1, MYB10, MYB100, MYB101, MYB102, MYB103, MYB104, MYB105, MYB106, MYB107, MYB108, MYB109, MYB11, MYB110, MYB111, MYB112, MYB113, MYB114, MYB115, MYB116, MYB117, MYB118, MYB119, MYB12, MYB120, MYB121, MYB122, MYB123 (TT2), MYB13, MYB14, MYB15, MYB16, MYB17, MYB18, MYB19, MYB2, MYB20, MYB21, MYB22, MYB23, MYB24, MYB25, MYB26, MYB27, MYB28, MYB29, MYB3, MYB30, MYB31, MYB32, MYB33, MYB330, MYB34, MYB35, MYB36, MYB37, MYB38, MYB39, MYB3R, MYB3R1, MYB3R2, MYB3R3, MYB3R4, MYB3R5, MYB4, MYB40, MYB41, MYB42, MYB43, MYB44, MYB45, MYB46, MYB47, MYB48, MYB49, MYB5, MYB50, MYB51, MYB52, MYB53, MYB54, MYB55, MYB56, MYB57, MYB58, MYB59, MYB6, MYB60, MYB61, MYB62, MYB63, MYB64, MYB65, MYB66, MYB67, MYB68, MYB69, MYB7, MYB70, MYB71, MYB72, MYB73, MYB74, MYB75 (PAP1 or PAPA1), MYB76, MYB77, MYB78, MYB79, MYB8, MYB80, MYB81, MYB82, MYB83, MYB84, MYB85, MYB86, MYB87, MYB88, MYB89, MYB9, MYB90 (PAP2 or PAPA2), MYB91, MYB92, MYB93, MYB94, MYB95, MYB96, MYB97, MYB98, MYB99, ANT1, AN2, A, MYB1a, P, C1, ROSEA1 (ROS1), ROSEA2 (ROS2), VENOSA, MBF1, MYBA, MYB, MYB110a, PURPLE (PR), GMYB10, VIMYBA1-1, VIMYBA1-2, VIMYBA1-3, VIMYBA2, MYBA1, MYBA2, MYBF1, MYBPA1, MYBPA2, MYB5a, MYB5b, MYBP3, MYBP4, LEGUME ANTHOCYANIN PRODUCTION1 (LAP1), PAR, MYB12-Lat, TT2a, TT2b, TT2c, PL, PL-BLOTCHED1 (PL-BH), P1, MYB-IF35, RUBY, DP, L, PHZ, MYB134, MYB216, AN1, MTF1, Y1, MYB176, MYB-G20-1, MYB12B2.

In one embodiment, the MYB type transcription factor is one or more of MYB1, MYB3, MYB10, MYB110a, MYBA, MYB11, MYB12, MYB14, MYB15, MYB46, MYB58, MYB61, MYB63, MYB75 (PAP1), MYB83, MYB85, MYB90 (PAP2), MYB111, MYB113, MYB114, MYB123 (TT2), MYB2, PURPLE (PR), GMYB10, VIMYBA1-1, VIMYBA1-2, VIMYBA1-3, VIMYBA2, MYBA1, MYBA2, MYBF1, MYBPA1, MYBPA2, MYB5a, MYB5b, MYBP3, MYBP4, MYB4, LEGUME ANTHOCYANIN PRODUCTION1 (LAP1), PAR, MYB6, MYB12-Lat, TT2a, TT2b, TT2c, C1, PL, PL-BLOTCHED1 (PL-BH), P1, MYB-IF35, RUBY, AN2, DP, L, PHZ, MYB20, MYB134, MYB216, AN1, MTF1, ROS1, ROS2, VENOSA, Y1, MYB176, MYB-G20-1, MYB12B2, MYB9, A, ANT1.

In one embodiment, the MYB type transcription factor is one or more of MYB0, MYB1, MYB10, MYB100, MYB101, MYB102, MYB103, MYB104, MYB105, MYB106, MYB107, MYB108, MYB109, MYB11, MYB110, MYB111, MYB112, MYB113, MYB114, MYB115, MYB116, MYB117, MYB118, MYB119, MYB12, MYB120, MYB121, MYB122, MYB123 (TT2), MYB13, MYB14, MYB15, MYB16, MYB17, MYB18, MYB19, MYB2, MYB20, MYB21, MYB22, MYB23, MYB24, MYB25, MYB26, MYB27, MYB28, MYB29, MYB3, MYB30, MYB31, MYB32, MYB33, MYB34, MYB35, MYB36, MYB37, MYB38, MYB39, MYB3R, MYB3R1, MYB3R2, MYB3R3, MYB3R4, MYB3R5, MYB4, MYB40, MYB41, MYB42, MYB43, MYB44, MYB45, MYB46, MYB47, MYB48, MYB49, MYB5, MYB50, MYB51, MYB52, MYB53, MYB54, MYB55, MYB56, MYB57, MYB58, MYB59, MYB6, MYB60, MYB61, MYB62, MYB63, MYB64, MYB65, MYB66, MYB67, MYB68, MYB69, MYB7, MYB70, MYB71, MYB72, MYB73, MYB74, MYB75 (PAP1 or PAPA1), MYB76, MYB77, MYB78, MYB79, MYB8, MYB80, MYB81, MYB82, MYB83, MYB84, MYB85, MYB86, MYB87, MYB88, MYB89, MYB9, MYB90 (PAP2 or PAPA2), MYB91, MYB92, MYB93, MYB94, MYB95, MYB96, MYB97, MYB98, MYB99, ANT1, AN2, A, MYB1a, P, C1, ROSEA1 (ROS1), ROSEA2 (ROS2), VENOSA, MBF1, MYBA, MYB, MYB110a, PURPLE (PR), GMYB10, VIMYBA1-1, VIMYBA1-2, VIMYBA1-3, VIMYBA2, MYBA1, MYBA2, MYBF1, MYBPA1, MYBPA2, MYB5a, MYB5b, MYBP3, MYBP4, LEGUME ANTHOCYANIN PRODUCTION1 (LAP1), PAR, MYB12-Lat, TT2a, TT2b, TT2c, PL, PL-BLOTCHED1 (PL-BH), P1, MYB-IF35, RUBY, DP, L, PHZ, MYB134, MYB216, AN1, MTF1, Y1, MYB176, MYB-G20-1, MYB12B2.

In one embodiment, the MYB type transcription factor is one or more of MYB1, MYB3, MYB10, MYB110a, MYBA, MYB11, MYB12, MYB14, MYB15, MYB46, MYB58, MYB61, MYB63, MYB75 (PAP1), MYB83, MYB85, MYB90 (PAP2), MYB111, MYB113, MYB114, MYB123 (TT2), MYB2, PURPLE (PR), GMYB10, VIMYBA1-1, VIMYBA1-2, VIMYBA1-3, VIMYBA2, MYBA1, MYBA2, MYBF1, MYBPA1, MYBPA2, MYB5a, MYB5b, MYBP3, MYBP4, MYB4, LEGUME ANTHOCYANIN PRODUCTION1 (LAP1), PAR, MYB6, MYB12-Lat, TT2a, TT2b, TT2c, C1, PL, PL-BLOTCHED1 (PL-BH), P1, MYB-IF35, RUBY, AN2, DP, L, PHZ, MYB20, MYB134, MYB216, AN1, MTF1, ROS1, ROS2, VENOSA, Y1, MYB176, MYB-G20-1, MYB12B2, MYB9, A, ANT1.

In one embodiment, the MYB is one or more of MYB75 (PAP1), MYB90 (PAP2), MYB113, MYB114, ANT1, AN2, A, MYB1a, P, C1, MYB1, ROSEA1 (ROS1), ROSEA2 (ROS2), VENOSA, MYB10, MBF1, MYBA, MYB330, TT2, MYB14, MYBPA1, MYB5b, and MYB.

In one embodiment, the MYB is one or more of MYB330, MYB12, MYB5a, AN2, and ROS1.

In one embodiment, the MYB is one or more of MYB330, MYB12, MYB5a, and AN2.

In one embodiment, the MYB type transcription factor is MYB12.

In one embodiment, the MYB type transcription factor is AtMYB12.

In one embodiment, the MYB type transcription factor is ROS1.

In one embodiment, the MYB type transcription factor is AmROS1.

In one embodiment, the MYB type transcription factor is AN2.

In one embodiment, the MYB type transcription factor is NtAN2.

In one embodiment, the MYB type transcription factor is MYB330.

In one embodiment, the MYB type transcription factor is NtMYB330.

In one embodiment, the MYB type transcription factor is PAP1.

In one embodiment, the MYB type transcription factor is AtPAP1.

In one embodiment, the MYB type transcription factor is TT2.

In one embodiment, the MYB type transcription factor is AtTT2.

In one embodiment, the MYB type transcription factor is MYB14.

In one embodiment, the MYB type transcription factor is MtMYB14.

In one embodiment, the MYB type transcription factor is MYBPA1.

In one embodiment, the MYB type transcription factor is VvMYBPA1.

In one embodiment, the MYB type transcription factor is MYB5b.

In one embodiment, the MYB type transcription factor is CsMYB5b.

In one embodiment, the MYB type transcription factor is MYB75.

In one embodiment, the MYB type transcription factor is CsMYB75.

In one embodiment, the MYB type transcription factor is MYB5a.

In one embodiment, the MYB type transcription factor is at least one of MYB12, AN2, ROS1, MYB330, PAP1, TT2, MYB14, MYBPA1, MYB5a, MYB5b, and MYB75.

In one embodiment, the MYB type transcription factor is at least one of MYB12, AN2, ROS1, MYB330, PAP1, TT2, MYB14, MYBPA1, MYB5b, and MYB75.

In one embodiment, the MYB type transcription factor is at least one of MYB12, MYB330, MYB14, MYBPA1, MYB5a, MYB5b, and MYB75.

In one embodiment, the MYB type transcription factor is at least one of MYB12, MYB330, MYB14, MYBPA1, MYB5b, and MYB75.

In one embodiment, the MYB type transcription factor is at least one of AtMYB12, NtAN2, AmROS1, NtMYB330, AtPAP1, AtTT2, MtMYB14, VvMYBPA1, CsMYB5b, and CsMYB75. bHLH type transcription factors are so named due to their highly conserved alkaline/helix-loop-helix domain. Originally identified in animals, they have also been found in plants and are involved in diverse functions including plant growth and development, via the functions of DNA binding and dimerization.

In one embodiment, the bHLH type transcription factor is one or more of Delila (DEL), Lc, TT8 (bHLH042), GL3 (bHLH001), EGL3 (bHLH002), MYC1 (bHLH012), MYC3 (bHLH005), MYC4 (bHLH004), bHLH1, bHLH003, bHLH007, MYC1, TT8, SIAN1, SIGL3, DEL, PPLS1, bHLH33, MYC2, bHLH25, bHLH29, bHLH80, bHLH98, MYC1, Ppb1, bHLH1, bHLH3, bHLH13, bHLH2, bHLH42.

In one embodiment, the bHLH type transcription factor is one or more of DEL, TT8, or TT9.

In a further embodiment, DEL, TT8, and TT9 are AmDEL, AtTT8, and AtTT9.

In one embodiment, the bHLH type transcription factor is DEL.

In one embodiment, the bHLH type transcription factor is AmDEL.

In one embodiment, the bHLH type transcription factor is TT8.

In one embodiment, the bHLH type transcription factor is AtTT8.

In one embodiment, the bHLH type transcription factor is TT9.

In one embodiment, the bHLH type transcription factor is AtTT9.

In one embodiment, the one or more transcription factors present in the flavonoid pathway are selected from the group comprising or consisting of TT8, TT9, AN2, MYB12, DEL, and ROS1.

In a further embodiment, TT8, TT9, AN2, MYB12, DEL, and ROS1 are AtTT8, AtTT9, NtAN2, AtMYB12, AmDEL, and AmROS1.

In one embodiment, the one or more transcription factors present in the flavonoid pathway are AN2, TT8, and MYB12.

In a further embodiment, AN2, TT8, and MYB12 are NtAN2, AtTT8, and AtMYB12.

In one embodiment, the one or more transcription factors present in the flavonoid pathway are AN2, TT9, and MYB12.

In a further embodiment, AN2, TT8, and MYB12 are NtAN2, AtTT9, and AtMYB12.

In one embodiment, the one or more transcription factors present in the flavonoid pathway are MYB12, DEL, and ROS1.

In a further embodiment, MYB12, DEL, and ROS1 are AtMYB12, AmDEL, and AmROS1.

In one embodiment, the one or more transcription factors present in the flavonoid pathway are DEL and ROS1.

In a further embodiment, DEL and ROS1 are AmDEL and AmROS1.

In one embodiment, the one or more transcription factors present in the flavonoid pathway are AN2 and TT8.

In a further embodiment, AN2 and TT8 are NtAN2 and AtTT8.

In one embodiment, the one or more transcription factors present in the flavonoid pathway are AN2 and TT9.

In a further embodiment, AN2 and TT9 are NtAN2 and AtTT9.

In one embodiment, the one or more transcription factors present in the flavonoid pathway are MYB12, DEL, ROS1, MYB330, PAP1, TT2, MYB14, MYBPA1, MYB5a, MYB5b, and MYB75.

In one embodiment, the one or more transcription factors present in the flavonoid pathway are MYB12, DEL, ROS1, MYB330, PAP1, TT2, MYB14, MYBPA1, MYB5b, and MYB75.

In a further embodiment, MYB12, DEL, ROS1, MYB330, PAP1, TT2, MYB14, MYBPA1, MYB5b, and MYB75, are AtMYB12, AmDEL, AmROS1, NtMYB330, AtPAP1, AtTT2, MtMYB14, VvMYBPA1, CsMYB5b, and CsMYB75.

In one embodiment, the one or more transcription factors present in the flavonoid pathway is MYB330.

In one embodiment, the one or more transcription factors present in the flavonoid pathway are MYB330 and MYB12.

In one embodiment, the one or more transcription factors present in the flavonoid pathway are MYB330, MYB12, and MYB5a.

In one embodiment, the one or more transcription factors present in the flavonoid pathway are MYB330, MYB12, MYB5a, and AN2.

In one embodiment, the one or more transcription factors present in the flavonoid pathway are MYB330, MYB12, MYB5a, AN2, DEL and ROS1.

The skilled person will understand that different plant species have their own transcription factors present in the flavonoid pathway. Therefore, the lists of transcription factors present in the flavonoid pathway provided above are non-limiting and the skilled person would select the most appropriate transcription factors based on the plant species of interest.

### Enzymes Present in the Flavonoid Pathway

As described herein, the composition for promoting the expression of one or more polyphenols optionally comprises one or more second vector components encoding one or more enzymes present in the flavonoid pathway.

In a further embodiment, the one or more enzymes present in the flavonoid pathway comprise one or more of the group comprising or consisting of a flavonoid pathway hydroxylase, O-methyltransferase (OMT), O-malonyltransferase, prenyltransferase, glucosyltransferase, lyase, ligase, synthase, isomerase, or dehydratase, reductase.

In one embodiment, the one or more second vector components encode two or more enzymes present in the flavonoid pathway.

In a further embodiment, the two or more enzymes present in the flavonoid pathway comprise two or more of the group comprising or consisting of a flavonoid pathway hydroxylase, O-methyltransferase (OMT), O-malonyltransferase, prenyltransferase, glucosyltransferase, lyase, ligase, synthase, isomerase, or dehydratase, reductase.

In one embodiment, the one or more second vector components encode three or more enzymes present in the flavonoid pathway.

In a further embodiment, the three or more enzymes present in the flavonoid pathway comprise three or more of the group comprising or consisting of a flavonoid pathway hydroxylase, O-methyltransferase (OMT), O-malonyltransferase, prenyltransferase, glucosyltransferase, lyase, ligase, synthase, isomerase, dehydratase, or reductase.

In one embodiment, the one or more second vector components encode four or more enzymes present in the flavonoid pathway.

In a further embodiment, the four or more enzymes present in the flavonoid pathway comprise four or more of the group comprising or consisting of a flavonoid pathway hydroxylase, O-methyltransferase (OMT), O-malonyltransferase, prenyltransferase, glucosyltransferase, lyase, ligase, synthase, isomerase, dehydratase, or reductase.

In one embodiment, the one or more second vector components encode five or more enzymes present in the flavonoid pathway.

In a further embodiment, the five or more enzymes present in the flavonoid pathway comprise five or more of the group comprising or consisting of a flavonoid pathway hydroxylase, O-methyltransferase (OMT), O-malonyltransferase, prenyltransferase, glucosyltransferase, lyase, ligase, synthase, isomerase, dehydratase, or reductase.

In one embodiment, the one or more second vector components encode 6, 7, 8, 9, 10, 11, 12, 13, 14, 15, 16, 17, 18, 19, 20, 21, 22, 23, 24, or 25 or more enzymes present in the flavonoid pathway.

In a further embodiment, the 6, 7, 8, 9, 10, 11, 12, 13, 14, 15, 16, 17, 18, 19, 20, 21, 22, 23, 24, or 25 or more enzymes present in the flavonoid pathway comprise 6, 7, 8, 9, 10, 11, 12, 13, 14, 15, 16, 17, 18, 19, 20, 21, 22, 23, 24, or 25 or more of the group comprising or consisting of a flavonoid pathway hydroxylase, O-methyltransferase (OMT), prenyltransferase, glucosyltransferase, lyase, ligase, synthase, isomerase, dehydratase, and/or reductase.

In one embodiment the hydroxylase is one or more of the group comprising or consisting of cinnamate 4-hydroxylase (C4H), flavanone 2-hydroxylase (F2H), flavanone 3-hydroxylase (F3H), flavanone 3′-hydroxylase (F3′H), flavonoid 3′,5′-hydroxylase (F3′,5′H), flavanone 3b-hydroxylase (FHT), and/or Flavone 8-hydroxylase (F8H).

In one embodiment, the OMT is one or more of the group comprising or consisting of kava O-methyltransferase 1 or 2 (KOMT1/KOMT2), OMT1, 8-OMT, OMT2884, OMT2, and/or OMT5.

In one embodiment, the prenyltransferase is one or more of the group comprising or consisting of APT(Orf2), NovQ, and/or naringenin 8-dimethylallyltransferase (N8DT-1).

In one embodiment, the glucosyltransferase is one or more of the group comprising or consisting of flavonoid 3-O-glucosyltransferase (3GT) and/or one or more of the UDP-glycosyltransferase (UGT) superfamily.

In a further embodiment, the UGT is either O, C or N linked.

In yet a further embodiment, the UGT is one or more of UDP-Glucose, UDP-Rutinose, UDP-Sophorose, UDP-Galactose, UDP-Xylose, UDP-Rhamnose, UDP-Sambubiose, UDP-Arabinose, UDP-Rhamnose, and/or UDP-Apiose.

In a further embodiment, the UGT is one or more of the group comprising or consisting of UGT30, UGT134, UGT73B2, UGT1A3, UGT1A4, UGT1A8, UGT2, UGT2B7, UGT3, UGT5, UGT71A8, UGT71A9, UGT71A10, UGT71B1, UGT71B6, UGT71K3, UGT72B14, UGT72E1, UGT72E2, UGT72E3, UGT72L1, UGT73B1, UGT73B6, UGT73G1, UGT73J1, UGT74F2, UGT74H6, UGT74S1, UGT75B1, UGT75B2, UGT76G1, UGT78D1, UGT78D2, UGT79B1, UGT84A2, UGT84A13, UGT84B2, UGT85C2, UGT85K14, UGT89C1, UGT94D1, UGT708C1, UGT709C2, UGT75C1, UGT81A1, UGT84A23 and/or UGT84A24.

In one embodiment, the lyase is one or more of the group comprising or consisting of phenylalanine ammonia lyase (PAL), and/or tyrosine ammonia lyase (TAL).

In one embodiment, the ligase is one or more of the group comprising or consisting of 4-coumarate: CoA ligase (4CL) and/or CoA ligase 7 (CLL-7).

In one embodiment, the synthase is one or more of the group comprising or consisting of chalcone synthase (CHS), anthocyanidin synthase (ANS), isoflavone synthase (IFS), styrylpyrone synthase (SPS), soluble flavone synthase (FNSI), membrane-bound flavone synthase (FNSII), and/or flavonol synthase (FLS).

In a further embodiment, CHS is CHS-2.

In a further embodiment, ANS is ANS1 or ANS2.

In a further embodiment, FNSII is FNSII-2.

In one embodiment, the isomerase is one or more of the group comprising or consisting of chalcone isomerase (CHI).

In one embodiment, the dehydratase is 2-hydroxyisoflavanone dehydratase (HID) In one embodiment, the reductase is one or more of the group comprising or consisting of chalcone reductase (CHR), dihydroflavonol 4-reductase (DFR), anthocyanidin reductase (ANR), and/or leucoanthocyanidin reductase (LAR).

The skilled person understands that enzymes present in the flavonoid pathway may be isolated from a variety of different plant species. Thus, a plurality of different isoforms may exist for each enzyme described herein.

In a further embodiment, the one or more flavonoid pathway enzymes comprise one or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the two or more flavonoid pathway enzymes comprise two or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the three or more flavonoid pathway enzymes comprise three or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the four or more flavonoid pathway enzymes comprise four or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24). In a further embodiment, the five or more flavonoid pathway enzymes comprise five or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the six or more flavonoid pathway enzymes comprise six or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the seven or more flavonoid pathway enzymes comprise seven or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the eight or more flavonoid pathway enzymes comprise eight or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the nine or more flavonoid pathway enzymes comprise nine or more of the group comprising or consisting of SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the ten or more flavonoid pathway enzymes comprise ten or more of the group comprising or consisting of SPS (1 and/or 2), SPS (1 and/or 2), KOMT (e.g. KOMT1), F3′,5′H, F3H, F2H, ANR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the 11, 12, 13, 14, 15, 16, 17, 18, 19, 20, 21, 22, 23, 24, or 25 or more flavonoid pathway enzymes comprise 11, 12, 13, 14, 15, 16, 17, 18, 19, 20, 21, 22, 23, 24, or 25 or more of the group comprising or consisting of SPS (1 and/or 2), KOMT (e.g. KOMT1 and/or KOMT2), F3′,5′H, F3H, F2H, ANR, IFS, HID, ANS (e.g. ANS1 and/or ANS2), OMT (e.g. OMT1, OMT1A, OMT1B, OMT2, OMT3, OMT4, OMT5, OMT6, OMT7, OMT8, OMT9, OMT10, OMT11, OMT12A, OMT12B, OMT13, OMT14, OMT2′, OMT3′, OMT5′, OMT6′, and/or OMT4″), CoA ligase (e.g. CLL-7), CHS (e.g. CHS-2), FNSII (e.g. FNSII-2), F8H, FNSI, or UGT (e.g. UGT84A23, UGT84A24. UGT30, UGT134, UGT73B2, UGT1A3, UGT1A4, UGT1A8, UGT2, UGT2B7, UGT3, UGT5, UGT71A8, UGT71A9, UGT71A10, UGT71B1, UGT71B6, UGT71K3, UGT72B14, UGT72E1, UGT72E2, UGT72E3, UGT72L1, UGT73B1, UGT73B6, UGT73G1, UGT73J1, UGT74F2, UGT74H6, UGT74S1, UGT75B1, UGT75B2, UGT76G1, UGT78D1, UGT78D2, UGT79B1, UGT84A2, UGT84A13, UGT84B2, UGT85C2, UGT85K14, UGT89C1, UGT94D1, UGT708C1, UGT709C2, UGT75C1, or UGT81A1).

In a further embodiment, the one or more flavonoid pathway enzymes comprise one or more of the group comprising or consisting of SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the two or more flavonoid pathway enzymes comprise two or more of the group comprising or consisting of SPS (1 and/or 2), KOMT (e.g. KOMT1), F3′,5′H, F3H, F2H, ANR, LAR IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the three or more flavonoid pathway enzymes comprise three or more of the group comprising or consisting of SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the four or more flavonoid pathway enzymes comprise four or more of the group comprising or consisting of SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the five or more flavonoid pathway enzymes comprise five or more of the group comprising or consisting of SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the six or more flavonoid pathway enzymes comprise six or more of the group comprising or consisting of SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the seven or more flavonoid pathway enzymes comprise seven or more of the group comprising or consisting of SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the eight or more flavonoid pathway enzymes comprise eight or more of the group comprising or consisting of SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the nine or more flavonoid pathway enzymes comprise nine or more of the group comprising or consisting of SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the ten or more flavonoid pathway enzymes comprise ten or more of the group comprising or consisting of SPS (1 and/or 2), SPS (1 and/or 2), KOMT (e.g. KOMT1), F3′,5′H, F3H, F2H, ANR, LAR, IFS, HID, ANS (e.g. ANS2), OMT (e.g. OMT1 and/or OMT5), CoA ligase (e.g. CLL-7), CHS (e.g. CHS-2), FNSII (e.g. FNSII-2), F8H, FNSI, UGT (e.g. UGT84A23, or UGT84A24).

In a further embodiment, the 11, 12, 13, 14, 15, 16, 17, 18, 19, 20, 21, 22, 23, 24, or 25 or more flavonoid pathway enzymes comprise 11, 12, 13, 14, 15, 16, 17, 18, 19, 20, 21, 22, 23, 24, or 25 or more of the group comprising or consisting of SPS (1 and/or 2), KOMT (e.g. KOMT1 and/or KOMT2), F3′,5′H, F3H, F2H, ANR, LAR, IFS, HID, ANS (e.g. ANS1 and/or ANS2), OMT (e.g. OMT1, OMT1A, OMT1B, OMT2, OMT3, OMT4, OMT5, OMT6, OMT7, OMT8, OMT9, OMT10, OMT11, OMT12A, OMT12B, OMT13, OMT14, OMT2′, OMT3′, OMT5′, OMT6′, and/or OMT4″), CoA ligase (e.g. CLL-7), CHS (e.g. CHS-2), FNSII (e.g. FNSII-2), F8H, FNSI, or UGT (e.g. UGT84A23, UGT84A24. UGT30, UGT134, UGT73B2, UGT1A3, UGT1A4, UGT1A8, UGT2, UGT2B7, UGT3, UGT5, UGT71A8, UGT71A9, UGT71A10, UGT71B1, UGT71B6, UGT71K3, UGT72B14, UGT72E1, UGT72E2, UGT72E3, UGT72L1, UGT73B1, UGT73B6, UGT73G1, UGT73J1, UGT74F2, UGT74H6, UGT74S1, UGT75B1, UGT75B2, UGT76G1, UGT78D1, UGT78D2, UGT79B1, UGT84A2, UGT84A13, UGT84B2, UGT85C2, UGT85K14, UGT89C1, UGT94D1, UGT708C1, UGT709C2, UGT75C1, or UGT81A1).

In one embodiment, the one or more flavonoid pathway enzymes is F3′,5′H.

In one embodiment, the one or more flavonoid pathway enzymes is F3H.

In one embodiment, the one or more flavonoid pathway enzymes is F2H.

In one embodiment, the one or more flavonoid pathway enzymes is ANR.

In one embodiment, the one or more flavonoid pathway enzymes is IFS.

In one embodiment, the one or more flavonoid pathway enzymes is HID.

In one embodiment, the one or more flavonoid pathway enzymes is ANS2.

In one embodiment, the one or more flavonoid pathway enzymes is OMT.

In a further embodiment, OMT is one or more of OMT1, OMT1A, OMT1B, OMT2, OMT3, OMT4, OMT5, OMT6, OMT7, OMT8, OMT9, OMT10, OMT11, OMT12A, OMT12B, OMT13, OMT14, OMT2′, OMT3′, OMT5′, OMT6′, and/or OMT4″.

In one embodiment, the one or more flavonoid pathway enzymes are SbCLL-7, SbCHS-2, FNSII-2, SbF8H, and SbOMT5.

In one embodiment, the one or more flavonoid pathway enzymes are FNSI, UGT84A23, and UGT84A24.

In one embodiment, the one or more flavonoid pathway enzymes are SPS (1 and/or 2) and KOMT1.

In one embodiment, the one or more flavonoid pathway enzymes are ANR and LAR.

In selecting the combination of flavonoid pathway enzymes, the skilled person will use their knowledge of the flavonoid pathway to select appropriate enzymes to reach the desired polyphenol compound.

Polyphenols Obtainable from the Composition

As has already been disclosed, polyphenols are a diverse group plant compounds that have bioactive activity, acting as antioxidants that limit damage caused to cells by free radicals. Polyphenols can be divided into a number of sub-groups, of which flavonoids are the largest group. Flavonoids can be further divided into isoflavones, neoflavonoids, chalcones, flavones, flavonols, flavanones, flavanonols, proanthocyanidins and anthocyanidins (Tsao. Nutrients. 2010, 2(12): 1231-1246). Therefore, polyphenols present a diverse range of commercially attractive compounds.

As described herein, the composition promotes the expression of one or more polyphenols.

In one embodiment, the one or more polyphenols comprise one or more flavonoids.

In a further embodiment, the one or more flavonoids comprise one or more plant-derived flavonoids.

In one embodiment, the one or more polyphenols comprise one or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the one or more polyphenols comprise one or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the one or more polyphenols comprise one or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the one or more polyphenols is kaempferol, or a derivative thereof.

In one embodiment, the one or more polyphenols is quercetin, or a derivative thereof.

In one embodiment, the one or more polyphenols is cyanidin, or a derivative thereof.

In one embodiment, the one or more polyphenols is catechin, or a derivative thereof.

In one embodiment, the one or more polyphenols is dieckol, or a derivative thereof.

In one embodiment, the one or more polyphenols is amento flavon, or a derivative thereof.

In one embodiment, the one or more polyphenols is isorhamnetin, or a derivative thereof.

In one embodiment, the one or more polyphenols is rhamnetin, or a derivative thereof.

In one embodiment, the one or more polyphenols is myricitrin, or a derivative thereof.

In one embodiment, the one or more polyphenols is apigenin-7-O-glucoside, or a derivative thereof.

In one embodiment, the one or more polyphenols is wogonin, or a derivative thereof.

In one embodiment, the one or more polyphenols is epigallocatechin, or a derivative thereof.

In one embodiment, the composition promotes the expression of two or more polyphenols.

In one embodiment, the two or more polyphenols comprise two or more flavonoids.

In a further embodiment, the two or more flavonoids comprise two or more plant-derived flavonoids.

In one embodiment, the two or more polyphenols comprise two or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the two or more polyphenols comprise two or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the two or more polyphenols comprise two or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the two or more polyphenols is kaempferol, or a derivative thereof and at least one other polyphenol.

In one embodiment, the two or more polyphenols is quercetin, or a derivative thereof and at least one other polyphenol.

In one embodiment, the two or more polyphenols is cyanidin, or a derivative thereof and at least one other polyphenol.

In one embodiment, the two or more polyphenols is catechin, or a derivative thereof and at least one other polyphenol.

In one embodiment, the two or more polyphenols is dieckol, or a derivative thereof and at least one other polyphenol.

In one embodiment, the two or more polyphenols is amento flavon, or a derivative thereof and at least one other polyphenol.

In one embodiment, the two or more polyphenols is isorhamnetin, or a derivative thereof and at least one other polyphenol.

In one embodiment, the two or more polyphenols is rhamnetin, or a derivative thereof and at least one other polyphenol.

In one embodiment, the two or more polyphenols is myricitrin, or a derivative thereof and at least one other polyphenol.

In one embodiment, the two or more polyphenols is apigenin-7-O-glucoside, or a derivative thereof and at least one other polyphenol.

In one embodiment, the two or more polyphenols is wogonin, or a derivative thereof and at least one other polyphenol.

In one embodiment, the two or more polyphenols is epigallocatechin, or a derivative thereof and at least one other polyphenol.

In one embodiment, the composition promotes the expression of three or more polyphenols.

In one embodiment, the three or more polyphenols comprise three or more flavonoids.

In a further embodiment, the three or more flavonoids comprise three or more plant-derived flavonoids.

In one embodiment, the three or more polyphenols comprise three or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the three or more polyphenols comprise three or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the three or more polyphenols comprise three or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the three or more polyphenols is kaempferol, or a derivative thereof and at least two other polyphenols.

In one embodiment, the three or more polyphenols is quercetin, or a derivative thereof and at least two other polyphenols.

In one embodiment, the three or more polyphenols is cyanidin, or a derivative thereof and at least two other polyphenols.

In one embodiment, the three or more polyphenols is catechin, or a derivative thereof and at least two other polyphenols.

In one embodiment, the three or more polyphenols is dieckol, or a derivative thereof and at least two other polyphenols.

In one embodiment, the three or more polyphenols is amento flavon, or a derivative thereof and at least two other polyphenols.

In one embodiment, the three or more polyphenols is isorhamnetin, or a derivative thereof and at least two other polyphenols.

In one embodiment, the three or more polyphenols is rhamnetin, or a derivative thereof and at least two other polyphenols.

In one embodiment, the three or more polyphenols is myricitrin, or a derivative thereof and at least two other polyphenols.

In one embodiment, the three or more polyphenols is apigenin-7-O-glucoside, or a derivative thereof and at least two other polyphenols.

In one embodiment, the three or more polyphenols is wogonin, or a derivative thereof and at least two other polyphenols.

In one embodiment, the three or more polyphenols is epigallocatechin, or a derivative thereof and at least two other polyphenols.

In one embodiment, the composition promotes the expression of four or more polyphenols.

In one embodiment, the four or more polyphenols comprise four or more flavonoids.

In a further embodiment, the four or more flavonoids comprise four or more plant-derived flavonoids.

In one embodiment, the four or more polyphenols comprise four or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the four or more polyphenols comprise four or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the four or more polyphenols comprise four or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the four or more polyphenols is kaempferol, or a derivative thereof and at least three other polyphenols.

In one embodiment, the four or more polyphenols is quercetin, or a derivative thereof and at least three other polyphenols.

In one embodiment, the four or more polyphenols is cyanidin, or a derivative thereof and at least three other polyphenols.

In one embodiment, the four or more polyphenols is catechin, or a derivative thereof and at least three other polyphenols.

In one embodiment, the four or more polyphenols is dieckol, or a derivative thereof and at least three other polyphenols.

In one embodiment, the four or more polyphenols is amento flavon, or a derivative thereof and at least three other polyphenols.

In one embodiment, the four or more polyphenols is isorhamnetin, or a derivative thereof and at least three other polyphenols.

In one embodiment, the four or more polyphenols is rhamnetin, or a derivative thereof and at least three other polyphenols.

In one embodiment, the four or more polyphenols is myricitrin, or a derivative thereof and at least three other polyphenols.

In one embodiment, the four or more polyphenols is apigenin-7-O-glucoside, or a derivative thereof and at least three other polyphenols.

In one embodiment, the four or more polyphenols is wogonin, or a derivative thereof and at least three other polyphenols.

In one embodiment, the four or more polyphenols is epigallocatechin, or a derivative thereof and at least three other polyphenols.

In one embodiment, the composition promotes the expression of five or more polyphenols.

In one embodiment, the five or more polyphenols comprise five or more flavonoids.

In a further embodiment, the five or more flavonoids comprise five or more plant-derived flavonoids.

In one embodiment, the five or more polyphenols comprise five or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the five or more polyphenols comprise five or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the five or more polyphenols comprise five or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the five or more polyphenols is kaempferol, or a derivative thereof and at least four other polyphenols.

In one embodiment, the five or more polyphenols is quercetin, or a derivative thereof and at least four other polyphenols.

In one embodiment, the five or more polyphenols is cyanidin, or a derivative thereof and at least four other polyphenols.

In one embodiment, the five or more polyphenols is catechin, or a derivative thereof and at least four other polyphenols.

In one embodiment, the five or more polyphenols is dieckol, or a derivative thereof and at least four polyphenols.

In one embodiment, the five or more polyphenols is amento flavon, or a derivative thereof and at least four other polyphenols.

In one embodiment, the five or more polyphenols is isorhamnetin, or a derivative thereof and at least four other polyphenols.

In one embodiment, the five or more polyphenols is rhamnetin, or a derivative thereof and at least four other polyphenols.

In one embodiment, the five or more polyphenols is myricitrin, or a derivative thereof and at least four other polyphenols.

In one embodiment, the five or more polyphenols is apigenin-7-O-glucoside, or a derivative thereof and at least four other polyphenols.

In one embodiment, the five or more polyphenols is wogonin, or a derivative thereof and at least four other polyphenols.

In one embodiment, the five or more polyphenols is epigallocatechin, or a derivative thereof and at least four other polyphenols.

In one embodiment, the composition promotes the expression of 6, 7, 8, 9, 10, 11, 12, 13, 14, 15, 16, 17 or more polyphenols.

In one embodiment, the 6, 7, 8, 9, 10, 11, 12, 13, 14, 15, 16, 17 or more polyphenols are 6, 7, 8, 9, 10, 11, 12, 13, 14, 15, 16, 17 or more flavonoids.

In a further embodiment, the 6, 7, 8, 9, 10, 11, 12, 13, 14, 15, 16, 17 or more flavonoids comprise 6, 7, 8, 9, 10, 11, 12, 13, 14, 15, 16, 17 or more plant-derived flavonoids.

In one embodiment, the 6, 7, 8, 9, 10, 11, 12, 13, 14, 15, 16, 17 or more polyphenols comprise 6, 7, 8, 9, 10, 11, 12, 13, 14, 15, 16, 17 or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the 6, 7, 8, 9, 10, 11, 12, 13, 14, 15, 16, 17 or more polyphenols comprise 6, 7, 8, 9, 10, 11, 12, 13, 14, 15, 16, 17 or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof and at least 0, 1, 2, 3, 4, 5 other polyphenols.

In one embodiment, the 6, 7, 8, 9, 10, 11, 12, 13, 14, 15, 16, 17 or more polyphenols comprise 6, 7, 8, 9, 10, 11, or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof and at least 0, 1, 2, 3, 4, 5, 6 other polyphenols.

In one embodiment, the 6, 7, 8, 9, 10, 11, 12, 13, 14, 15, 16, 17 or more polyphenols is kaempferol, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is quercetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is cyanidin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is catechin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is dieckol, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is amento flavon, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is isorhamnetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is rhamnetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is myricitrin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is apigenin-7-O-glucoside, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is wogonin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is epigallocatechin, or a derivative thereof and at least 5, 6, 7, 8, 9, 10, 11, 12, 13, 14, 15, 16 other polyphenols.

The skilled person will understand that due to the nature of catalysis reactions, not all intermediate metabolites will be catalysed to form the next metabolite. The selected combinations of flavonoid pathway enzymes and transcription factors present in the flavonoid pathway will promote the expression of particular polyphenols and/or flavonoids. However, intermediate metabolites of the pathway may also be present. Therefore, a plurality of polyphenols and/or flavonoid metabolites may be expressed in the cell transfected with the vector described herein.

In certain embodiments, the polyphenol obtained and the combination of enzymes and transcription factors present in the flavonoid pathway to obtain the polyphenol are as recited in Table 1.

TABLE 1
provides non-limiting examples of combinations
of enzymes and transcription factors present in the
flavonoid pathway, encoded by the vector components as
described herein, used to obtain polyphenols and/or flavonoids.

| | Example enzyme-transcription |
|---|---|
| | factor combinations |
| Polyphenol obtained | encoded by the vector(s) |
| Helichrysin A | SPS(1 and/or 2), KOMT1, MYB12, |
| | Del, ROS or |
| | MYB12, SPS2, KOMT1 |
| Neochanin (Formononetin) | SPS(1 and/or 2), KOMT1, MYB12, |
| | Del, ROS or |
| | MYB12, SPS1, KOMT1 |
| Pinocembrin | SPS(1 and/or 2), KOMT1, MYB12, |
| | Del, ROS or |
| | MYB12, SPS1, KOMT1 |
| Sissotrin | SPS(1 and/or 2), KOMT1, MYB12, |
| | Del, ROS or |
| | MYB12, Del, Ros, SPS2, KOMT1 |
| Fustin | MYB12, SPS2, KOMT1 |
| genistein 7-O-beta-D-glucoside | SPS1, KOMT1, MYB12, Del, ROS |
| myricitrin | MYB12, SPS2, KOMT1 |
| Myricitrin 2′-O-gallate | AN2 |
| neohesperidin | Del, Ros |
| Neoeriocitrin | Del, Ros, SPS2, KOMT1 |
| Naringenin | MYB12, Del, Ros, SPS2, KOMT1 |
| Ononin | MYB12, SPS2, KOMT1 |
| Salipurposid | MYB12, SPS1, KOMT1 |
| Scutellarein | MYB12, SPS2, KOMT1 |
| Vicenin-2 | MYB12, SPS2, KOMT1 |
| quercetin 3-O-(6-O-malonyl- | MYB12, SPS2, KOMT1 |
| beta-D-glucoside) | |
| quercetin | MYB12, DEL, ROS1 |
| | or |
| | MYB12, AN2, TT8 |
| | or |
| | MYB12, SPS2, KOMT1 |
| Taxifolin (dihydroquercetin) | MYB12, SPS2, KOMT1 |
| Quercetin 3-O-glucoside | MYB12, SPS2, KOMT1 |
| Quercetin-3-methyl ether | MYB12, Del, Ros |
| trifolin | MYB12, Del, Ros |
| Kaempferol-3-O-rutinoside | MYB12, Del, Ros |
| (nicotiflorin) | |
| ramnazin-3-O-rutinoside | MYB12 |
| viscidulin I | MYB12, Del, Ros |
| isoquercitrin | MYB12, Del, Ros |
| Dihydrokaempferol | SPS2, KOMT1, MYB12, Del, ROS |
| kaempferol | MYB12, DEL, ROS1 |
| | or |
| | MYB12, AN2, TT9 |
| Kaempferol-3-O-galactoside | Del, Ros, MYB12 |
| quercitrin | MYB12, Del, Ros |
| isorhamnetin | OMT1, MYB12, Del, ROS |
| wogonin | SbCLL-7, SbCHS-2, FNSII-2, SbF8H, |
| | SbOMT5, MYB12, Del, ROS |
| apigenin-7-O-glucoside | FNSI, UGT84A23, UGT84A24, |
| | MYB12, Del, ROS or |
| | FNSI, UGT84A23, UGT84A24, |
| cyanidin | MYB12 DEL, ROS1 |
| | or |
| | AN2, TT8 |
| catechin | MYB12, Del, ROS, NtMYB330, |
| | AtPAP1, AtTT2, MtMYB14, |
| | VvMYBPA1, CsMYB5b, CsMYB75, |
| | VvMYBPA1 |
| | or |
| | ANR, LAR, DEL, ROS |
| | or |
| | ANR, LAR, DEL, ROS, MYB5a |
| | or |
| | ANR, LAR, DEL, ROS, MYB330 |
| | or |
| | ANR, LAR, DEL, ROS, AN2 |
| | or |
| | ANR, LAR, DEL, ROS, MYB330, |
| | MYB12, MYB5a, AN2 |

Phenylpropanoids

| | |
|---|---|
| 3,4-Dicaffeoylquinic acid | MYB12, SPS2, KOMT1 |
| 4-Hydroxy-3- | MYB12, Del, Ros |
| methoxycinnamaldehyde | |
| 5-O-Caffeoylshikimic acid | Del, Ros, SPS1, KOMT1 |
| Aesculin \| Esculin | MYB12 |
| Caffeic acid | AN2, TT8 |
| Chlorogenic acid | Del, Ros, SPS1, KOMT1 |
| Coniferin | Del, Ros, SPS1, KOMT1 |
| Coumarinic acid-beta-D- | MYB12, AN2 |
| glucoside | |
| Esculetin | MYB12, Del, Ros |
| Ferulic acid | MYB12 |
| Fraxetin | MYB12 |
| Fraxin | MYB12 |
| Neochlorogenic acid | Del, Ros, SPS1, KOMT1 |
| o-Coumaric acid | MYB12, Del, Ros, SPS1, KOMT1 |
| p-Coumaric acid | MYB12, Del, Ros, SPS1, KOMT1 |
| Phlorizin | MYB12, SPS1, KOMT1 |
| Rutarin | Del, Ros, SPS2, KOMT1 |
| Scoparone | Del, Ros |
| Scopoletin | Del, Ros |
| Sinapic acid | Del, Ros, SPS1, KOMT1 |
| Sinapoyl aldehyde | AN2 |
| Syringin | MYB12, TT8 |
| Umbelliferone | Del, Ros, SPS1, KOMT1 |
| Epigallocatechin | Del, Ros, ANR, LAR |
| | or |
| | Del, Ros, ANR, LAR, MYB330, |
| | MYB12, MYB5a, AN2 |

As is also exemplified in table 1, it is clear that there are combinations of enzymes and transcription factors present in the flavonoid pathway that are common to multiple polyphenols. Thus, a selected combination of enzymes and transcription factors present in the flavonoid pathway encoded by the vector components, has the potential to promote the expression of a plurality of polyphenols.

### Plant Products

As described herein, in one aspect there is provided a plant, plant part or plant cell transformed with the vector components according to any one of the composition embodiments described herein.

In one embodiment, the plant, plant part or plant cell is algae, moss or any plant, plant part or plant cell derived from the genus Nicotiana.

In a further embodiment, the plant, plant part or plant cell derived from the genus Nicotiana is selected from the group comprising or consisting of Nicotiana tabacum, Nicotiana benthamiana, or BY-2 cells.

### USE

As described herein, in one aspect there is provided use of the composition according to any one of the composition embodiments described herein for promoting the expression of one or more polyphenols in algae, moss, or any plant, plant part, or plant cells.

In one embodiment, the plant cells are a cell line derived from plants.

In one embodiment, the plant, plant part, or plant cells are derived from the genus Nicotiana.

In yet a further embodiment, the plant, plant part or plant cells derived from the genus Nicotiana are selected from the group comprising or consisting of Nicotiana tabacum, Nicotiana benthamiana, or BY-2 cells.

In an alternative embodiment, the expression of one or more polyphenols is promoted in yeast cells and/or bacterial cells.

In one embodiment, the one or more polyphenols comprise one or more flavonoids.

In a further embodiment, the one or more flavonoids comprise one or more plant-derived flavonoids.

In one embodiment, the one or more polyphenols comprise one or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the one or more polyphenols comprise one or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the one or more polyphenols comprise one or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the one or more polyphenols is kaempferol, or a derivative thereof.

In one embodiment, the one or more polyphenols is quercetin, or a derivative thereof.

In one embodiment, the one or more polyphenols is cyanidin, or a derivative thereof.

In one embodiment, the one or more polyphenols is catechin, or a derivative thereof.

In one embodiment, the one or more polyphenols is dieckol, or a derivative thereof.

In one embodiment, the one or more polyphenols is amento flavon, or a derivative thereof.

In one embodiment, the one or more polyphenols is isorhamnetin, or a derivative thereof.

In one embodiment, the one or more polyphenols is rhamnetin, or a derivative thereof.

In one embodiment, the one or more polyphenols is myricitrin, or a derivative thereof.

In one embodiment, the one or more polyphenols is apigenin-7-O-glucoside, or a derivative thereof.

In one embodiment, the one or more polyphenols is wogonin, or a derivative thereof.

In one embodiment, the one or more polyphenols is epigallocatechin, or a derivative thereof.

In one embodiment, the composition promotes the expression of two or more polyphenols.

In one embodiment, the two or more polyphenols comprise two or more flavonoids.

In a further embodiment, the two or more flavonoids comprise two or more plant-derived flavonoids.

In one embodiment, the two or more polyphenols comprise two or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the two or more polyphenols comprise two or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the two or more polyphenols comprise two or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the two or more polyphenols is kaempferol, or a derivative thereof and at least one other polyphenol.

In one embodiment, the two or more polyphenols is quercetin, or a derivative thereof and at least one other polyphenol.

In one embodiment, the two or more polyphenols is cyanidin, or a derivative thereof and at least one other polyphenol.

In one embodiment, the two or more polyphenols is catechin, or a derivative thereof and at least one other polyphenol.

In one embodiment, the two or more polyphenols is dieckol, or a derivative thereof and at least one other polyphenol.

In one embodiment, the two or more polyphenols is amento flavon, or a derivative thereof and at least one other polyphenol.

In one embodiment, the two or more polyphenols is isorhamnetin, or a derivative thereof and at least one other polyphenol.

In one embodiment, the two or more polyphenols is rhamnetin, or a derivative thereof and at least one other polyphenol.

In one embodiment, the two or more polyphenols is myricitrin, or a derivative thereof and at least one other polyphenol.

In one embodiment, the two or more polyphenols is apigenin-7-O-glucoside, or a derivative thereof and at least one other polyphenol.

In one embodiment, the two or more polyphenols is wogonin, or a derivative thereof and at least one other polyphenol.

In one embodiment, the two or more polyphenols is epigallocatechin, or a derivative thereof and at least one other polyphenols.

In one embodiment, the composition promotes the expression of three or more polyphenols.

In one embodiment, the three or more polyphenols comprise three or more flavonoids.

In a further embodiment, the three or more flavonoids comprise three or more plant-derived flavonoids.

In one embodiment, the three or more polyphenols comprise three or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the three or more polyphenols comprise three or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the three or more polyphenols comprise three or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the three or more polyphenols is kaempferol, or a derivative thereof and at least two other polyphenols.

In one embodiment, the three or more polyphenols is quercetin, or a derivative thereof and at least two other polyphenols.

In one embodiment, the three or more polyphenols is cyanidin, or a derivative thereof and at least two other polyphenols.

In one embodiment, the three or more polyphenols is catechin, or a derivative thereof and at least two other polyphenols.

In one embodiment, the three or more polyphenols is dieckol, or a derivative thereof and at least two other polyphenols.

In one embodiment, the three or more polyphenols is amento flavon, or a derivative thereof and at least two other polyphenols.

In one embodiment, the three or more polyphenols is isorhamnetin, or a derivative thereof and at least two other polyphenols.

In one embodiment, the three or more polyphenols is rhamnetin, or a derivative thereof and at least two other polyphenols.

In one embodiment, the three or more polyphenols is myricitrin, or a derivative thereof and at least two other polyphenols.

In one embodiment, the three or more polyphenols is apigenin-7-O-glucoside, or a derivative thereof and at least two other polyphenols.

In one embodiment, the three or more polyphenols is wogonin, or a derivative thereof and at least two other polyphenols.

In one embodiment, the three or more polyphenols is epigallocatechin, or a derivative thereof and at least two other polyphenols.

In one embodiment, the composition promotes the expression of four or more polyphenols.

In one embodiment, the four or more polyphenols comprise four or more flavonoids.

In a further embodiment, the four or more flavonoids comprise four or more plant-derived flavonoids.

In one embodiment, the four or more polyphenols comprise four or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the four or more polyphenols comprise four or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the four or more polyphenols comprise four or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the four or more polyphenols is kaempferol, or a derivative thereof and at least three other polyphenols.

In one embodiment, the four or more polyphenols is quercetin, or a derivative thereof and at least three other polyphenols.

In one embodiment, the four or more polyphenols is cyanidin, or a derivative thereof and at least three other polyphenols.

In one embodiment, the four or more polyphenols is catechin, or a derivative thereof and at least three other polyphenols.

In one embodiment, the four or more polyphenols is dieckol, or a derivative thereof and at least three other polyphenols.

In one embodiment, the four or more polyphenols is amento flavon, or a derivative thereof and at least three other polyphenols.

In one embodiment, the four or more polyphenols is isorhamnetin, or a derivative thereof and at least three other polyphenols.

In one embodiment, the four or more polyphenols is rhamnetin, or a derivative thereof and at least three other polyphenols.

In one embodiment, the four or more polyphenols is myricitrin, or a derivative thereof and at least three other polyphenols.

In one embodiment, the four or more polyphenols is apigenin-7-O-glucoside, or a derivative thereof and at least three other polyphenols.

In one embodiment, the four or more polyphenols is wogonin, or a derivative thereof and at least three other polyphenols.

In one embodiment, the four or more polyphenols is epigallocatechin, or a derivative thereof and at least three other polyphenols.

In one embodiment, the composition promotes the expression of five or more polyphenols.

In one embodiment, the five or more polyphenols comprise five or more flavonoids.

In a further embodiment, the five or more flavonoids comprise five or more plant-derived flavonoids.

In one embodiment, the five or more polyphenols comprise five or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the five or more polyphenols comprise five or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the five or more polyphenols comprise five or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the five or more polyphenols is kaempferol, or a derivative thereof and at least four other polyphenols.

In one embodiment, the five or more polyphenols is quercetin, or a derivative thereof and at least four other polyphenols.

In one embodiment, the five or more polyphenols is cyanidin, or a derivative thereof and at least four other polyphenols.

In one embodiment, the five or more polyphenols is catechin, or a derivative thereof and at least four other polyphenols.

In one embodiment, the five or more polyphenols is dieckol, or a derivative thereof and at least four polyphenols.

In one embodiment, the five or more polyphenols is amento flavon, or a derivative thereof and at least four other polyphenols.

In one embodiment, the five or more polyphenols is isorhamnetin, or a derivative thereof and at least four other polyphenols.

In one embodiment, the five or more polyphenols is rhamnetin, or a derivative thereof and at least four other polyphenols.

In one embodiment, the five or more polyphenols is myricitrin, or a derivative thereof and at least four other polyphenols.

In one embodiment, the five or more polyphenols is apigenin-7-O-glucoside, or a derivative thereof and at least four other polyphenols.

In one embodiment, the five or more polyphenols is wogonin, or a derivative thereof and at least four other polyphenols.

In one embodiment, the five or more polyphenols is epigallocatechin, or a derivative thereof and at least four other polyphenols.

In one embodiment, the composition promotes the expression of 6, 7, 8, 9, 10, 11, 12, 13, 14, 15, 16, 17 or more polyphenols.

In one embodiment, the 6, 7, 8, 9, 10, 11, 12, 13, 14, 15, 16, 17 or more polyphenols comprise 6, 7, 8, 9, 10, 11, 12, 13, 14, 15, 16, 17 or more flavonoids.

In a further embodiment, the 6, 7, 8, 9, 10, 11, 12, 13, 14, 15, 16, 17 or more flavonoids comprise 6, 7, 8, 9, 10, 11, 12, 13, 14, 15, 16, 17 or more plant-derived flavonoids.

In one embodiment, the 6, 7, 8, 9, 10, 11, 12, 13, 14, 15, 16, 17 or more polyphenols comprise 6, 7, 8, 9, 10, 11, 12, 13, 14, 15, 16, 17 or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the 6, 7, 8, 9, 10, 11, 12, 13, 14, 15, 16, 17 or more polyphenols comprise 6, 7, 8, 9, 10, 11, 12, 13, 14, 15, 16, 17 or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof and at least 0, 1, 2, 3, 4, 5 other polyphenols.

In one embodiment, the 6, 7, 8, 9, 10, 11, 12, 13, 14, 15, 16, 17 or more polyphenols comprise 6, 7, 8, 9, 10, 11, or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof and at least 0, 1, 2, 3, 4, 5, 6 other polyphenols.

In one embodiment, the 6, 7, 8, 9, 10, 11, 12, 13, 14, 15, 16, 17 or more polyphenols is kaempferol, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is quercetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is cyanidin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is catechin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is dieckol, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is amento flavon, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is isorhamnetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is rhamnetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is myricitrin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is apigenin-7-O-glucoside, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is wogonin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is epigallocatechin, or a derivative thereof and at least 5, 6, 7, 8, 9, 10, 11, 12, 13, 14, 15, 16 other polyphenols.

### METHOD

As described herein, in one aspect there is provided a method of promoting the expression of one or more polyphenols in a cell, comprising the step of:
a) introducing the composition according to any one of the composition embodiments described herein into a cell.

In one embodiment, the method further comprises:
b) extracting the one or more polyphenols from the cell to produce an extract.

In an additional embodiment, the method further comprises:
c) detecting the one or more polyphenols in the extract.

In an additional embodiment, the method further comprises:
d) purifying the one or more polyphenols from the extract.

In one embodiment, the method further comprises one or more of:
b) extracting the one or more polyphenols from the cell to produce an extract;
c) detecting the one or more polyphenols in the extract; and
d) purifying the one or more polyphenols from the extract.

In one embodiment, the method further comprises:
b) extracting the one or more polyphenols from the plant to produce an extract;
c) detecting the one or more polyphenols in the extract; and
d) purifying the one or more polyphenols from the extract.

In one embodiment, wherein the cell is part of a plant, the method further comprises: between step (a) and step (b), harvesting one or more leaves from the plant and treating the one or more leaves by curing and/or hydrolysis.

In a further embodiment, the hydrolysis is acid hydrolysis and/or enzymatic hydrolysis.

In yet a further embodiment, the enzymatic hydrolysis uses Pectinase, β-Glucosidase and/or Glucosidase.

The inventors have found that curing and/or treating the harvested leaves by hydrolysis, the amount of some polyphenols (such as kaempferol or quercetin) extracted from the cell is increased.

In a further embodiment, the one or more polyphenols are purified using Flash and preparative MPLC, HPTLC or LC/MS.

In one embodiment, the cell is a plant cell, yeast cell and/or bacteria cell.

In a further embodiment, the plant cell is an algae cell, moss cell, or any cell or cell line derived from the genus Nicotiana.

In yet a further embodiment, the cell or cell line derived from the genus Nicotiana is selected from the group comprising or consisting of Nicotiana tabacum, Nicotiana benthamiana, or BY-2 cells.

In one embodiment, the one or more polyphenols comprise one or more flavonoids.

In a further embodiment, the one or more flavonoids comprise one or more plant-derived flavonoids.

In one embodiment, the one or more polyphenols comprise one or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the one or more polyphenols comprise one or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the one or more polyphenols comprise one or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the one or more polyphenols is kaempferol, or a derivative thereof.

In one embodiment, the one or more polyphenols is quercetin, or a derivative thereof.

In one embodiment, the one or more polyphenols is cyanidin, or a derivative thereof.

In one embodiment, the one or more polyphenols is catechin, or a derivative thereof.

In one embodiment, the one or more polyphenols is dieckol, or a derivative thereof.

In one embodiment, the one or more polyphenols is amento flavon, or a derivative thereof.

In one embodiment, the one or more polyphenols is isorhamnetin, or a derivative thereof.

In one embodiment, the one or more polyphenols is rhamnetin, or a derivative thereof.

In one embodiment, the one or more polyphenols is myricitrin, or a derivative thereof.

In one embodiment, the one or more polyphenols is apigenin-7-O-glucoside, or a derivative thereof.

In one embodiment, the one or more polyphenols is wogonin, or a derivative thereof.

In one embodiment, the one or more polyphenols is epigallocatechin, or a derivative thereof.

In one embodiment, the composition promotes the expression of two or more polyphenols.

In one embodiment, the two or more polyphenols comprise two or more flavonoids.

In a further embodiment, the two or more flavonoids comprise two or more plant-derived flavonoids.

In one embodiment, the two or more polyphenols comprise two or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the two or more polyphenols comprise two or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the two or more polyphenols comprise two or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the two or more polyphenols is kaempferol, or a derivative thereof and at least one other polyphenol.

In one embodiment, the two or more polyphenols is quercetin, or a derivative thereof and at least one other polyphenol.

In one embodiment, the two or more polyphenols is cyanidin, or a derivative thereof and at least one other polyphenol.

In one embodiment, the two or more polyphenols is catechin, or a derivative thereof and at least one other polyphenol.

In one embodiment, the two or more polyphenols is dieckol, or a derivative thereof and at least one other polyphenol.

In one embodiment, the two or more polyphenols is amento flavon, or a derivative thereof and at least one other polyphenol.

In one embodiment, the two or more polyphenols is isorhamnetin, or a derivative thereof and at least one other polyphenol.

In one embodiment, the two or more polyphenols is rhamnetin, or a derivative thereof and at least one other polyphenol.

In one embodiment, the two or more polyphenols is myricitrin, or a derivative thereof and at least one other polyphenol.

In one embodiment, the two or more polyphenols is apigenin-7-O-glucoside, or a derivative thereof and at least one other polyphenol.

In one embodiment, the two or more polyphenols is wogonin, or a derivative thereof and at least one other polyphenol.

In one embodiment, the two or more polyphenols is epigallocatechin, or a derivative thereof and at least one other polyphenol.

In one embodiment, the composition promotes the expression of three or more polyphenols.

In one embodiment, the three or more polyphenols comprise three or more flavonoids.

In a further embodiment, the three or more flavonoids comprise three or more plant-derived flavonoids.

In one embodiment, the three or more polyphenols comprise three or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the three or more polyphenols comprise three or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the three or more polyphenols comprise three or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the three or more polyphenols is kaempferol, or a derivative thereof and at least two other polyphenols.

In one embodiment, the three or more polyphenols is quercetin, or a derivative thereof and at least two other polyphenols.

In one embodiment, the three or more polyphenols is cyanidin, or a derivative thereof and at least two other polyphenols.

In one embodiment, the three or more polyphenols is catechin, or a derivative thereof and at least two other polyphenols.

In one embodiment, the three or more polyphenols is dieckol, or a derivative thereof and at least two other polyphenols.

In one embodiment, the three or more polyphenols is amento flavon, or a derivative thereof and at least two other polyphenols.

In one embodiment, the three or more polyphenols is isorhamnetin, or a derivative thereof and at least two other polyphenols.

In one embodiment, the three or more polyphenols is rhamnetin, or a derivative thereof and at least two other polyphenols.

In one embodiment, the three or more polyphenols is myricitrin, or a derivative thereof and at least two other polyphenols.

In one embodiment, the three or more polyphenols is apigenin-7-O-glucoside, or a derivative thereof and at least two other polyphenols.

In one embodiment, the three or more polyphenols is wogonin, or a derivative thereof and at least two other polyphenols.

In one embodiment, the three or more polyphenols is epigallocatechin, or a derivative thereof and at least two other polyphenols.

In one embodiment, the composition promotes the expression of four or more polyphenols.

In one embodiment, the four or more polyphenols comprise four or more flavonoids.

In a further embodiment, the four or more flavonoids comprise four or more plant-derived flavonoids.

In one embodiment, the four or more polyphenols comprise four or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the four or more polyphenols comprise four or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the four or more polyphenols comprise four or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the four or more polyphenols is kaempferol, or a derivative thereof and at least three other polyphenols.

In one embodiment, the four or more polyphenols is quercetin, or a derivative thereof and at least three other polyphenols.

In one embodiment, the four or more polyphenols is cyanidin, or a derivative thereof and at least three other polyphenols.

In one embodiment, the four or more polyphenols is catechin, or a derivative thereof and at least three other polyphenols.

In one embodiment, the four or more polyphenols is dieckol, or a derivative thereof and at least three other polyphenols.

In one embodiment, the four or more polyphenols is amento flavon, or a derivative thereof and at least three other polyphenols.

In one embodiment, the four or more polyphenols is isorhamnetin, or a derivative thereof and at least three other polyphenols.

In one embodiment, the four or more polyphenols is rhamnetin, or a derivative thereof and at least three other polyphenols.

In one embodiment, the four or more polyphenols is myricitrin, or a derivative thereof and at least three other polyphenols.

In one embodiment, the four or more polyphenols is apigenin-7-O-glucoside, or a derivative thereof and at least three other polyphenols.

In one embodiment, the four or more polyphenols is wogonin, or a derivative thereof and at least three other polyphenols.

In one embodiment, the four or more polyphenols is epigallocatechin, or a derivative thereof and at least three other polyphenols.

In one embodiment, the composition promotes the expression of five or more polyphenols.

In one embodiment, the five or more polyphenols comprise five or more flavonoids.

In a further embodiment, the five or more flavonoids comprise five or more plant-derived flavonoids.

In one embodiment, the five or more polyphenols comprise five or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the five or more polyphenols comprise five or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the five or more polyphenols comprise five or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof.

In one embodiment, the five or more polyphenols is kaempferol, or a derivative thereof and at least four other polyphenols.

In one embodiment, the five or more polyphenols is quercetin, or a derivative thereof and at least four other polyphenols.

In one embodiment, the five or more polyphenols is cyanidin, or a derivative thereof and at least four other polyphenols.

In one embodiment, the five or more polyphenols is catechin, or a derivative thereof and at least four other polyphenols.

In one embodiment, the five or more polyphenols is dieckol, or a derivative thereof and at least four polyphenols.

In one embodiment, the five or more polyphenols is amento flavon, or a derivative thereof and at least four other polyphenols.

In one embodiment, the five or more polyphenols is isorhamnetin, or a derivative thereof and at least four other polyphenols.

In one embodiment, the five or more polyphenols is rhamnetin, or a derivative thereof and at least four other polyphenols.

In one embodiment, the five or more polyphenols is myricitrin, or a derivative thereof and at least four other polyphenols.

In one embodiment, the five or more polyphenols is apigenin-7-O-glucoside, or a derivative thereof and at least four other polyphenols.

In one embodiment, the five or more polyphenols is wogonin, or a derivative thereof and at least four other polyphenols.

In one embodiment, the five or more polyphenols is epigallocatechin, or a derivative thereof and at least four other polyphenols.

In one embodiment, the composition promotes the expression of 6, 7, 8, 9, 10, 11, 12, 13, 14, 15, 16, 17 or more polyphenols.

In one embodiment, the 6, 7, 8, 9, 10, 11, 12, 13, 14, 15, 16, 17 or more polyphenols are 6, 7, 8, 9, 10, 11, 12, 13, 14, 15, 16, 17 or more flavonoids.

In a further embodiment, the 6, 7, 8, 9, 10, 11, 12, 13, 14, 15, 16, 17 or more flavonoids comprise 6, 7, 8, 9, 10, 11, 12, 13, 14, 15, 16, 17 or more plant derived flavonoids.

In one embodiment, the 6, 7, 8, 9, 10, 11, 12, 13, 14, 15, 16, 17 or more polyphenols comprise 6, 7, 8, 9, 10, 11, 12, 13, 14, 15, 16, 17 or more of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the 6, 7, 8, 9, 10, 11, 12, 13, 14, 15, 16, 17 or more polyphenols comprise 6, 7, 8, 9, 10, 11, 12, 13, 14, 15, 16, 17 or more of the group comprising or consisting of kaempferol, quercetin, cyanidin, catechin, dieckol, amento flavon, isorhamnetin, rhamnetin, myricitrin, apigenin-7-O-glucoside, wogonin, and epigallocatechin, or derivatives thereof.

In one embodiment, the 6, 7, 8, 9, 10, 11, 12, 13, 14, 15, 16, 17 or more polyphenols comprise 6, 7, 8, 9, 10, 11, or more of the group comprising or consisting of kaempferol, quercetin, cyanidin, catechin, dieckol, amento flavon, isorhamnetin, rhamnetin, myricitrin, apigenin-7-O-glucoside, and wogonin, or derivatives thereof and at least 0, 1, 2, 3, 4, 5, 6 other polyphenols.

In one embodiment, the 6, 7, 8, 9, 10, 11, 12, 13, 14, 15, 16, 17 or more polyphenols is kaempferol, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is quercetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is cyanidin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is catechin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is dieckol, or a derivative thereof and at least 5, 6, 7, 8, 9, 10, 11, 12, 13, 14, 15, 16 other polyphenols. In one embodiment, the 6, 7, 8, 9, 10, 11, 12, 13, 14, 15, 16, 17 or more polyphenols is amento flavon, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is isorhamnetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is rhamnetin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is myricitrin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is apigenin-7-O-glucoside, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is wogonin, or a derivative thereof and at least 5, 6, 7, 8, 9, 10, 11, 12, 13, 14, 15, 16 other polyphenols.

In one embodiment, the 6, 7, 8, 9, 10, 11, 12, 13, 14, 15, 16, 17 or more polyphenols is epigallocatechin, or a derivative thereof and at least 5, 6, 7, 8, 9, 10, 11, 12, 13, 14, 15, 16 other polyphenols.

### Products

As described herein, in one aspect there is provided a polyphenol obtainable from the method according to any one of the method embodiments described herein.

In one embodiment, the polyphenol is a flavonoid.

In a further embodiment, the flavonoid is a plant-derived flavonoid.

In one embodiment, the polyphenol is any one of the group comprising or consisting of myricitrin, neohesperidin, quercetin 3-O-(6-O-malonyl-beta-D-glucoside), quercetin, trifolin, nicotiflorin, ramnazin-3-O-rutinoside, viscidulin I, isoquercitrin, dihydrokaempferol, kaempferol, quercitrin, isorhamnetin, wogonin, apigenin-7-O-glucoside, cyanidin, catechin, pinocembrin chalcone, eriodictyol chalcone, hydroxy-methoxy chalcone, 2-hydroxy-methoxy chalcone, Hydroxy-2 methoxy chalcone, naringenin chalcone, isoliquiritigenin, genistein, daidzein, eriodictyol, luteolin, apigenin, myricetin, pelargonidin, delphindin, afzelechin, gallocatechin, epiafzelechin, epigallocatechin, epicatechin, proanthocyanidins, dieckol, amento flavon, rhamnetin, apigenin-7-O-glucoside, or derivatives thereof.

In one embodiment, the polyphenol is kaempferol, or a derivative thereof.

In one embodiment, the polyphenol is quercetin, or a derivative thereof.

In one embodiment, the polyphenol is cyanidin, or a derivative thereof.

In one embodiment, the polyphenol is catechin, or a derivative thereof.

In one embodiment, the polyphenol is myricitrin, or a derivative thereof.

In one embodiment, the polyphenol is neohesperidin, or a derivative thereof.

In one embodiment, the polyphenol is quercetin 3-O-(6-O-malonyl-beta-D-glucoside), or a derivative thereof.

In one embodiment, the polyphenol is trifolin, or a derivative thereof.

In one embodiment, the polyphenol is nicotiflorin, or a derivative thereof.

In one embodiment, the polyphenol is ramnazin-3-O-rutinoside, or a derivative thereof. In one embodiment, the polyphenol is viscidulin I, or a derivative thereof.

In one embodiment, the polyphenol is isoquercitrin, or a derivative thereof.

In one embodiment, the polyphenol is dihydrokaempferol, or a derivative thereof.

In one embodiment, the polyphenol is quercitrin, or a derivative thereof.

In one embodiment, the polyphenol is isorhamnetin, or a derivative thereof.

In one embodiment, the polyphenol is wogonin, or a derivative thereof.

In one embodiment, the polyphenol is apigenin-7-O-glucoside, or a derivative thereof.

In one embodiment, the polyphenol is pinocembrin chalcone, or a derivative thereof.

In one embodiment, the polyphenol is eriodictyol chalcone, or a derivative thereof.

In one embodiment, the polyphenol is hydroxy-methoxy chalcone, or a derivative thereof.

In one embodiment, the polyphenol is 2-hydroxy-methoxy chalcone, or a derivative thereof.

In one embodiment, the polyphenol is Hydroxy-2 methoxy chalcone, or a derivative thereof.

In one embodiment, the polyphenol is naringenin chalcone, or a derivative thereof.

In one embodiment, the polyphenol is isoliquiritigenin, or a derivative thereof.

In one embodiment, the polyphenol is genistein, or a derivative thereof.

In one embodiment, the polyphenol is daidzein, or a derivative thereof.

In one embodiment, the polyphenol is eriodictyol, or a derivative thereof.

In one embodiment, the polyphenol is luteolin, or a derivative thereof.

In one embodiment, the polyphenol is apigenin, or a derivative thereof.

In one embodiment, the polyphenol is myricetin, or a derivative thereof.

In one embodiment, the polyphenol is pelargonidin, or a derivative thereof.

In one embodiment, the polyphenol is delphindin, or a derivative thereof.

In one embodiment, the polyphenol is afzelechin, or a derivative thereof.

In one embodiment, the polyphenol is gallocatechin, or a derivative thereof.

In one embodiment, the polyphenol is epiafzelechin, or a derivative thereof.

In one embodiment, the polyphenol is epigallocatechin, or a derivative thereof.

In one embodiment, the polyphenol is epicatechin, or a derivative thereof.

In one embodiment, the polyphenol is proanthocyanidins, or a derivative thereof.

In one embodiment, the polyphenol is dieckol, or a derivative thereof.

In one embodiment, the polyphenol is amento flavon, or a derivative thereof.

In one embodiment, the polyphenol is rhamnetin, or a derivative thereof.

In one embodiment, the polyphenol is apigenin-7-O-glucoside, or a derivative thereof.

### Vector Combinations

As described herein, different combinations of transcription factors present in the flavonoid pathway and optionally enzymes present in the flavonoid pathway result in increased expression of different polyphenols.

There now follows non-limiting examples of different compositions designed to promote the expression of one or more polyphenols.

In one embodiment, there is provided a composition for promoting the expression of one or more polyphenols in a cell, wherein the composition comprises:
a) a first vector component encoding MYB12

In a further embodiment, the composition comprising a first vector component encoding MYB12 promotes the expression of ramnazin-3-O-rutinoside, aesculin/esculin, ferulic acid, fraxetin, and fraxin.

In yet a further embodiment MYB12 is AtMYB12.

In one embodiment, there is provided a composition for promoting the expression of one or more polyphenols in a cell, wherein the composition comprises:
a) a first vector component encoding MYB12, DEL and ROS

In a further embodiment, the composition comprising a first vector component encoding MYB12, DEL and ROS promotes the expression of quercetin, kaempferol, quercetin-3-methyl ether, trifolin, kaempferol-3-O-rutinoside (nicotiflorin), viscidulin I, isoquercitrin, kaempferol-3-O-galactoside, quercitrin, 4-Hydroxy-3-methoxycinnamaldehyde, and esculetin.

In yet a further embodiment MYB12, DEL and ROS are AtMYB12, AmDEL and AmROS1.

In one embodiment, there is provided a composition for promoting the expression of one or more polyphenols in a cell, wherein the composition comprises:
a) a first vector component encoding MYB12, TT8, and AN2.

In a further embodiment, the composition comprising a first vector component encoding MYB12, TT8 and AN2, promotes the expression of quercetin.

In yet a further embodiment, MYB12, TT8 and AN2 are AtMYB12, AtTT8 and NtAN2.

In one embodiment, there is provided a composition for promoting the expression of one or more polyphenols in a cell, wherein the composition comprises:
a) a first vector component encoding MYB12, TT9, and AN2.

In a further embodiment, the composition comprising a first vector component encoding MYB12, TT9 and AN2, promotes the expression of kaempferol.

In yet a further embodiment, MYB12, TT9 and AN2 are AtMYB12, AtTT9 and NtAN2.

In one embodiment, there is provided a composition for promoting the expression of one or more polyphenols in a cell, wherein the composition comprises:
a) a first vector component encoding DEL and ROS.

In a further embodiment, the composition comprising a first vector component encoding DEL and ROS promotes the expression of catechin, cyanidin, scoparone, and scopoletin.

In yet a further embodiment, DEL and ROS are AmDEL and AmROS1.

In one embodiment, there is provided a composition for promoting the expression of one or more polyphenols in a cell, wherein the composition comprises:
a) a first vector component encoding TT8, and AN2.

In a further embodiment, the composition comprising a first vector component encoding TT8 and AN2 promotes the expression of cyanidin and caffeic acid.

In yet a further embodiment, TT8 and AN2 are AtTT8 and NtAN2.

In one embodiment, there is provided a composition for promoting the expression of one or more polyphenols in a cell, wherein the composition comprises:
a) a first vector component encoding TT9, and AN2.

In a further embodiment, the composition comprising a first vector component encoding TT9 and AN2 promotes the expression of catechin.

In yet a further embodiment, TT9 and AN2 are AtTT9 and NtAN2.

In one embodiment, there is provided a composition for promoting the expression of one or more polyphenols in a cell, wherein the composition comprises:
a) a first vector component encoding MYB12, DEL, ROS; and
b) a second vector component encoding OMT1.

In a further embodiment, the composition comprising a first vector component encoding MYB12, DEL, ROS and a second vector component encoding OMT1 promotes the expression of Isorhamnetin.

In yet a further embodiment MYB12, DEL, ROS, and OMT1 are AtMYB12, AmDEL, AmROS1, and OMT1.

In one embodiment, there is provided a composition for promoting the expression of one or more polyphenols in a cell, wherein the composition comprises:
a) a first vector component encoding MYB12, DEL, ROS; and
b) a second vector component encoding SbCLL-7, SbCHS-2, FNSII-2, SbF8H, SbOMT5.

In a further embodiment, the composition comprising a first vector component encoding MYB, DEL, ROS and a second vector component encoding SbCLL-7, SbCHS-2, FNSII-2, SbF8H, and SbOMT5 promotes the expression of wogonin.

In yet a further embodiment MYB12, DEL, ROS and CLL-7, CHS-2, FNSII-2, F8H, OMT5 are AtMYB12, AmDEL, AmROS1, and SbCLL-7, SbCHS-2, (any one of ObFNSII-2, GFNSII-2, OsFNSII-2), SbF8H, SbOMT5.

In one embodiment, there is provided a composition for promoting the expression of one or more polyphenols in a cell, wherein the composition comprises:
a) a first vector component encoding MYB12, DEL, ROS; and
b) a second vector component encoding FNSI, UGT84A23, UGT84A24.

In a further embodiment, the composition comprising a first vector component encoding MYB12, DEL, ROS and a second vector component encoding FNSI, UGT84A23, UGT84A24 promotes the expression of Apigenin-7-O-glucoside.

In yet a further embodiment MYB12, DEL, ROS and FNSI, UGT84A23, UGT84A24 are AtMYB12, AmDEL, AmROS1 and FNSI, UGT84A23, UGT84A24.

In one embodiment, there is provided a composition for promoting the expression of one or more polyphenols in a cell, wherein the composition comprises:
a) a first vector component encoding MYB12, DEL, ROS; and
b) a second vector component encoding SPS1, OMT1

In a further embodiment, the composition comprising a first vector component encoding MYB12, DEL, ROS and a second vector component encoding SPS1, OMT1 promotes the expression of helichrysin A, neochanin (formononetin), pinocembrin, sissotrin, genistein 7-O-beta-D-glucoside, o-coumaric acid, and p-coumaric acid.

In yet a further embodiment MYB12, DEL, ROS and SPS1, OMT1 are AtMYB12, AmDEL, AmROS1 and SPS1, KOMT1.

In one embodiment, there is provided a composition for promoting the expression of one or more polyphenols in a cell, wherein the composition comprises:
a) a first vector component encoding MYB12, DEL, ROS; and
b) a second vector component encoding SPS2, OMT1

In a further embodiment, the composition comprising a first vector component encoding MYB12, DEL, ROS and a second vector component encoding SPS2, OMT1, promotes the expression of helichrysin A, neochanin (formononetin), pinocembrin, sissotrin, naringenin, and dihydrokaempferol.

In yet a further embodiment MYB12, DEL, ROS and SPS2, OMT1 are AtMYB12, AmDEL, AmROS1 and SPS2, KOMT1.

In one embodiment, there is provided a composition for promoting the expression of one or more polyphenols in a cell, wherein the composition comprises:
a) a first vector component encoding MYB12; and
b) a second vector component encoding SPS1, OMT1.

In a further embodiment, the composition comprising a first vector component encoding MYB12 and a second vector component encoding SPS1, OMT1 promotes the expression of neochanin (formononetin), pinocembrin, salipurposid, and phlorizin.

In yet a further embodiment MYB12 and SPS1, OMT1 are AtMYB12 and SPS1, KOMT1.

In one embodiment, there is provided a composition for promoting the expression of one or more polyphenols in a cell, wherein the composition comprises:
a) a first vector component encoding MYB12; and
b) a second vector component encoding SPS2, OMT1.

In a further embodiment, the composition comprising a first vector component encoding MYB12 and a second vector component encoding SPS2, OMT1 promotes the expression of helichrysin A, fustin, myricitrin, ononin, scutellarein, vicenin-2, quercetin 3-O-(6-O-malonyl-beta-D-glucoside), quercetin, taxifolin (dihydroquercetin), quercetin 3-O-glucoside, and 3,4-dicaffeoylquinic acid.

In yet a further embodiment, MYB12 and SPS2, OMT1 are AtMYB12 and SPS2, KOMT1.

In one embodiment, there is provided a composition for promoting the expression of one or more polyphenols in a cell, wherein the composition comprises:
a) a first vector component encoding AN2

In a further embodiment, the composition comprising a first vector component encoding AN2 promotes the expression of myricitrin 2′-O-gallate and sinapoyl aldehyde.

In yet a further embodiment, AN2 is NtAN2.

In one embodiment, there is provided a composition for promoting the expression of one or more polyphenols in a cell, wherein the composition comprises:
a) a first vector component encoding DEL, ROS; and
b) a second vector component encoding SPS1, OMT1.

In a further embodiment, the composition comprises a first vector component encoding DEL, ROS and a second vector component encoding SPS1, OMT1 promotes the expression of 5-O-caffeoylshikimic acid, chlorogenic acid, coniferin, neochlorogenic acid, sinapic acid, and umbelliferone.

In yet a further embodiment, DEL, ROS and SPS1, OMT1 are AmDEL, AmROS1 and SPS1, KOMT1.

In one embodiment, there is provided a composition for promoting the expression of one or more polyphenols in a cell, wherein the composition comprises:
a) a first vector component encoding DEL, ROS; and
b) a second vector component encoding SPS2, OMT1.

In a further embodiment, the composition comprises a first vector component encoding DEL, ROS and a second vector component encoding SPS2, OMT1 promotes the expression of neoeriocitrin and rutarin.

In yet a further embodiment, DEL, ROS and SPS2, OMT1 are AmDEL, AmROS1 and SPS2, KOMT1.

In one embodiment, there is provided a composition for promoting the expression of one or more polyphenols in a cell, wherein the composition comprises:
a) a first vector component encoding MYB12, DEL, ROS, MYB330, PAP1, TT2, MYB14, MYBPA1, MYB5b, and MYB75.

In a further embodiment, the composition comprises a first vector component encoding MYB12, DEL, ROS, MYB330, PAP1, TT2, MYB14, MYBPA1, MYB5b, and MYB75 promotes the expression of catechin.

In yet a further embodiment, MYB12, DEL, ROS, MYB330, PAP1, TT2, MYB14, MYBPA1, MYB5b, and MYB75 are AtMYB12, AmDEL, AmROS, NtMYB330, AtPAP1, AtTT2, MtMYB14, VvMYBPA1, CsMYB5b, and CsMYB75.

In one embodiment, there is provided a composition for promoting the expression of one or more polyphenols in a cell, wherein the composition comprises:
a) a first vector component encoding MYB12 and AN2.

In a further embodiment, the composition comprises a first vector component encoding MYB12 and AN2 promotes the expression of coumarinic acid-beta-D-glucoside.

In yet a further embodiment, MYB12 and AN2 are AtMYB12 and NtAN2.

In one embodiment, there is provided a composition for promoting the expression of one or more polyphenols in a cell, wherein the composition comprises:
a) a first vector component encoding MYB12 and TT8.

In a further embodiment, the composition comprises a first vector component encoding MYB12 and TT8 promotes the expression of syringin.

In yet a further embodiment, MYB12 and TT8 are AtMYB12 and AtTT8.

In one embodiment, there is provided a composition for promoting the expression of one or more polyphenols in a cell, wherein the composition comprises:
a) a first vector component encoding DEL and ROS; and
b) a second vector component encoding ANR and LAR.

In a further embodiment, the composition comprising a first vector component encoding DEL and ROS, and a second vector component encoding ANR and LAR, promotes the expression of one or more of epicatechin, catechin, epigallocatechin and pelargonidin, or a derivative thereof.

In one embodiment, there is provided a composition for promoting the expression of one or more polyphenols in a cell, wherein the composition comprises:
a) a first vector component encoding MYB330 and MYB12.

In a further embodiment, the composition comprising a first vector component encoding MYB330 and MYB12 promotes the expression of pelargonidin, or a derivative thereof.

In one embodiment, there is provided a composition for promoting the expression of one or more polyphenols in a cell, wherein the composition comprises:
a) a first vector component encoding MYB330, MYB12, and MYB5a.

In a further embodiment, the composition comprising a first vector component encoding MYB330, MYB12, and MYB5a, promotes the expression of pelargonidin, or a derivative thereof.

In one embodiment, there is provided a composition for promoting the expression of one or more polyphenols in a cell, wherein the composition comprises:
a) a first vector component encoding MYB330, MYB12, MYB5a, and AN2.

In a further embodiment, the composition comprising a first vector component encoding MYB330, MYB12, MYB5a, and AN2, promotes the expression of pelargonidin, or a derivative thereof.

In one embodiment, there is provided a composition for promoting the expression of one or more polyphenols in a cell, wherein the composition comprises:
a) a first vector component encoding MYB330, MYB12, MYB5a, and AN2; and
b) a second vector component encoding ANR and LAR.

In a further embodiment, the composition comprising a first vector component encoding MYB330, MYB12, MYB5a, and AN2 and a second vector component encoding ANR and LAR, promotes the expression of pelargonidin, or a derivative thereof.

In one embodiment, there is provided a composition for promoting the expression of one or more polyphenols in a cell, wherein the composition comprises:
a) a first vector component encoding DEL, ROS, MYB330, MYB12, MYB5a, and AN2; and
b) a second vector component encoding ANR and LAR.

In a further embodiment, the composition comprising a first vector component encoding DEL, ROS, MYB330, MYB12, MYB5a, and AN2, and a second vector component encoding ANR and LAR, promotes the expression of epigallocatechin, or a derivative thereof.

In one embodiment, there is provided a composition for promoting the expression of one or more polyphenols in a cell, wherein the composition comprises:
a) a first vector component encoding DEL, ROS, and MYB330; and
b) a second vector component encoding ANR and LAR.

In a further embodiment, the composition comprising a first vector component encoding DEL, ROS, and MYB330, and a second vector component encoding ANR and LAR, promotes the expression of catechin, epicatechin, and epigallocatechin, or a derivative thereof.

In one embodiment, there is provided a composition for promoting the expression of one or more polyphenols in a cell, wherein the composition comprises:
a) a first vector component encoding DEL, ROS, and MYB12; and
b) a second vector component encoding ANR and LAR.

In a further embodiment, the composition comprising a first vector component encoding DEL, ROS, and MYB12 and a second vector component encoding ANR and LAR, promotes the expression of epigallocatechin, or a derivative thereof.

In one embodiment, there is provided a composition for promoting the expression of one or more polyphenols in a cell, wherein the composition comprises:
a) a first vector component encoding DEL, ROS, and MYB5a; and
b) a second vector component encoding ANR and LAR.

In a further embodiment, the composition comprising a first vector component encoding DEL, ROS, and MYB5a, and a second vector component encoding ANR and LAR, promotes the expression of catechin, epicatechin, and epigallocatechin, or a derivative thereof.

In one embodiment, there is provided a composition for promoting the expression of one or more polyphenols in a cell, wherein the composition comprises:
a) a first vector component encoding DEL, ROS, and AN2; and
b) a second vector component encoding ANR and LAR.

In a further embodiment, the composition comprising a first vector component encoding DEL, ROS, and AN2, and a second vector component encoding ANR and LAR, promotes the expression of epigallocatechin, or a derivative thereof.

In one embodiment, there is provided a composition for promoting the expression of one or more polyphenols in a cell, wherein the composition comprises:
a) a first vector component encoding MYB12; and
b) a second vector component encoding FNSI, UGT84A23, and UGT84A24.

In a further embodiment, the composition comprising a first vector component encoding MYB12 and a second vector component encoding FNSI, UGT84A23, and UGT84A24, promotes the expression of apigenin-7-O-glucoside, or a derivative thereof.

In one embodiment, there is provided a composition for promoting the expression of one or more polyphenols in a cell, wherein the composition comprises:
a) a first vector component encoding MYB12, DEL, and ROS; and
b) a second vector component encoding FNSI, UGT84A23, and UGT84A24.

In a further embodiment, the composition comprising a first vector component encoding MYB12, DEL, and ROS, and a second vector component encoding FNSI, UGT84A23, and UGT84A24, promotes the expression of apigenin-7-O-glucoside, or a derivative thereof.

The skilled person will understand that the lists of polyphenols, the expression of which may be promoted by the disclosed combinations of enzymes and/or transcription factors, are not limiting and may, for example, also include polyphenols that are promoted by one, two, three or more components of the disclosed combination. For example: MYB12 promotes the expression of fraxin. Therefore, the combination of MYB12, DEL and ROS may also promote fraxin, in addition to the list of polyphenols disclosed for that specific combination.

The expressed polyphenols, may or may not be endogenously present in the cell, cell line, yeast, bacteria, algae, moss, or plant in which the composition of the invention is used. It will be understood by the skilled person that where a particular polyphenol is not endogenously produced, there is considered to be an increase in this polyphenol if it can be detected after the cell, cell line, yeast, bacteria, algae, moss or plant has been transfected with the composition of the invention.

In situations where particular polyphenols are endogenously expressed in the cell, cell line, yeast, bacteria, algae, moss, or plant before the composition of the invention is used, a 1-fold, 2-fold, 3-fold, 4-fold, 5-fold, 10-fold, 20-fold, 40-fold, 60-fold, 80-fold, 100-fold, 150-fold, 200-fold, 300-fold, 400-fold, 600-fold, 800-fold, 1000-fold, or more increase in that particular polyphenol may be observed.

The total amount of polyphenols present in a cell, cell line, yeast, bacteria, algae, moss, or plant in which the composition of the invention is used may be increased by 1-fold, 2-fold, 3-fold, 4-fold, 5-fold, 10-fold, 20-fold, 40-fold, 60-fold, 80-fold, 100-fold, 150-fold, 200-fold, 250-fold, or more.

The various aspects and/or embodiments described herein are presented only to assist in understanding and teaching the claimed features. These aspects and/or embodiments are provided as a representative sample of aspects and/or embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

The invention will now be described with reference to the following non-limiting examples.

### EXAMPLES

### Example 1: Promoting the Expression of Polyphenols in Nicotiana tabacum

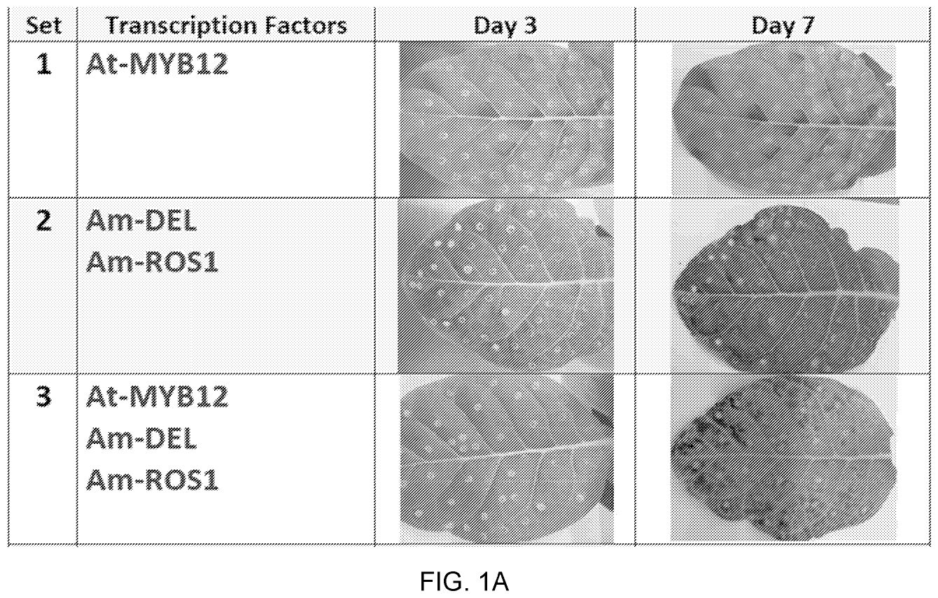

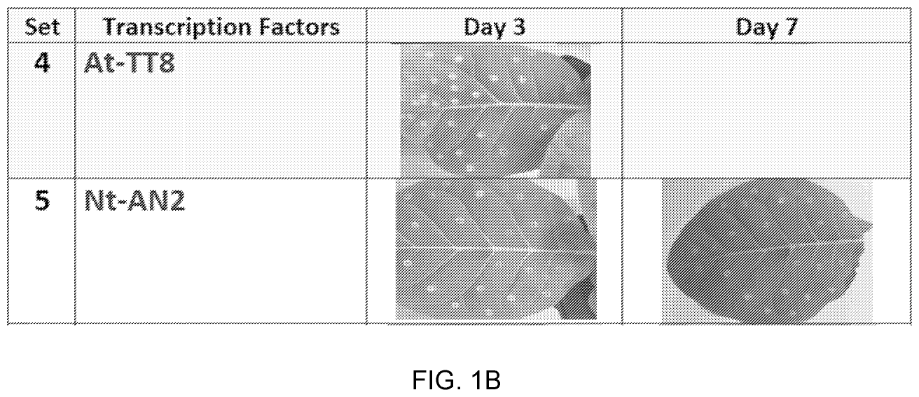

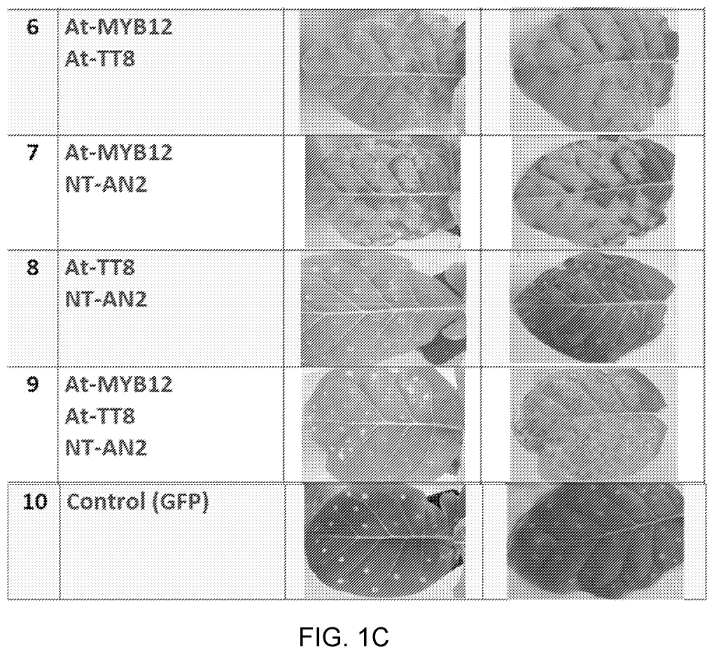

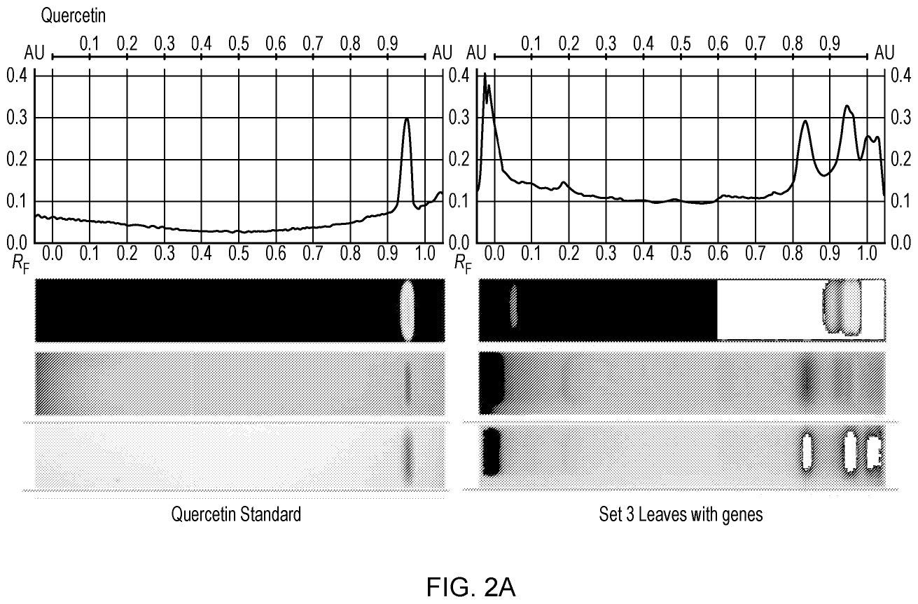

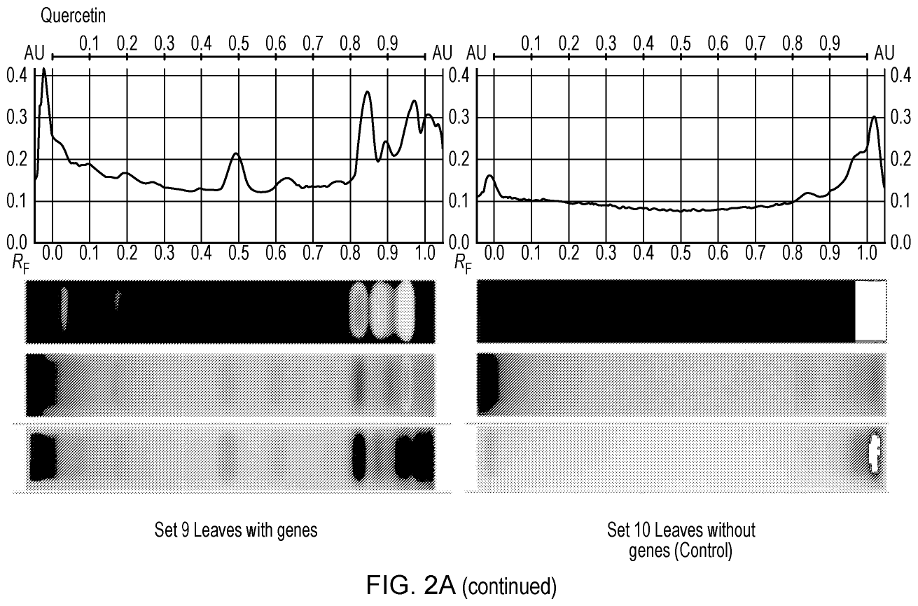

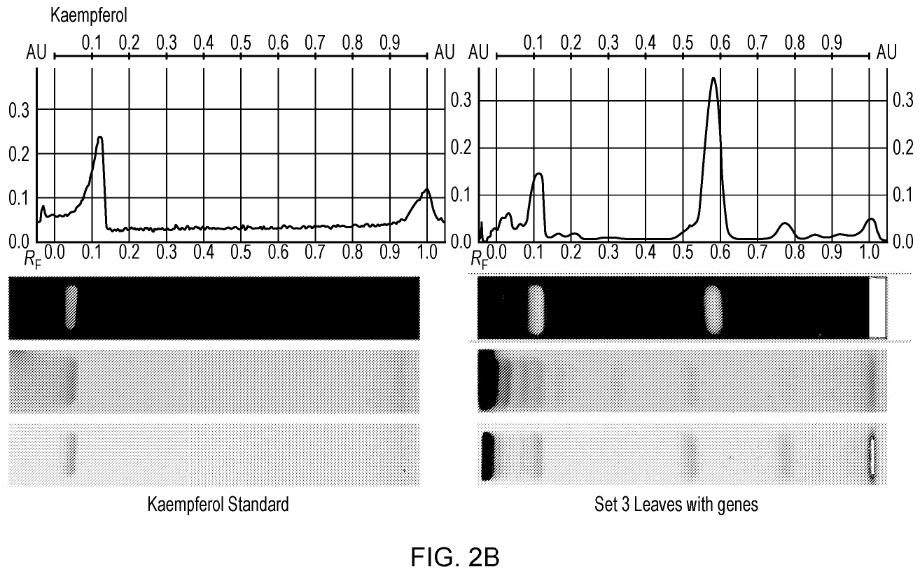

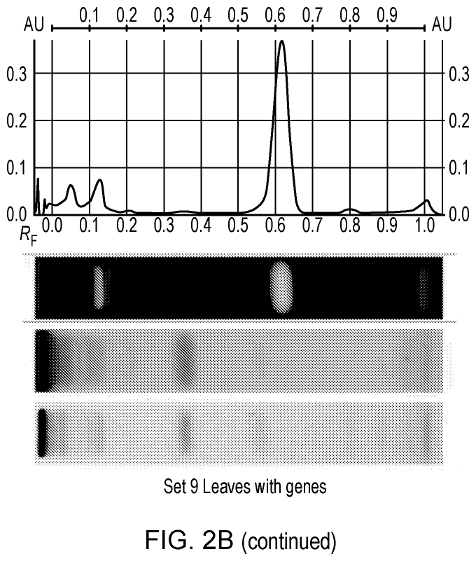

Plasmids comprising combinations of AtTT8, AmDEL, AmROS1, NtAN2, and AtMYB12 were generated and used to transfect Nicotiana tabacum. The combinations were as set out in table 2 below, which corresponds to the transfected leaves in FIG. 1 and the HPTLC results in FIG. 2.

### Table 2: Combinations of Transcription Factors in the Vectors of Sets 1-9.

TABLE 2
Combinations of transcription factors in the vectors of sets 1-9.

| Set | Transcription Factors |
|---|---|
| 1 | AtMYB12 |
| 2 | AmDEL, AmROS1 |
| 3 | AtMYB12, AmDEL, AmROS1 |
| 4 | AtTT8 |
| 5 | NtAN2 |
| 6 | AtTT8, AtMYB12 |
| 7 | AtMYB12, NtAN2 |
| 8 | AtTT8, NtAN2 |
| 9 | AtTT9, AtMYB12, NtAN2 |
| 10 | Control GFP |

An Agrobacterium culture (Agrobacterium tumefaciens) was used to deliver the genetic material into the Nicotiana tabacum cells. The plasmids containing the genes of interest were introduced into separate Agrobacterium cultures and cultured overnight to reach specific density.

Once the specific density was reached (OD600 of ~1-2), it was resuspended at a density of OD600 0.2 and prepared as an infiltration solution. Infiltration solutions were prepared for each of the plasmid types (see table 2).

The infiltration solutions were then individually infiltrated into the leaves of the Nicotiana tabacum plants, using a syringe or vacuum infiltration. Separate plants were used for each set of transcription factors.

Following infiltration, the plants were incubated under controlled conditions (22 °C, ~250 μmol/m2/s (light), 70% humidity and 16 hr day/night cycle) in a growth chamber for 7 days. Following this, the infiltrated leaves were removed from the plant and frozen instantly.

The dried leaves were then ground and extraction was preformed using methanol. Extracts were then filtered and placed in vials to be analysed by HPTLC. Stationary Phase; Merck, HPTLC Silica gel 60 F₂₅₄, Mobile phase; Chloroform:Methanol 9:1 (v/v), Chloroform:Methanol:Water 80:20:2 (v/v/v), Chloroform:Methanol:Water 61:32:5 (v/v/v), Toluene:Ethyl acetate:Acetic acid:Chloroform 5:5:1:2 (v/v/v/v). After separation, the plates were derivatized with 10% H2SO4 in methanol and then visualized by normal light or UV lamp.

Table 3: Fold Change in Polyphenols after Transfection with the Vectors According to Table 2.

TABLE 3
fold change in polyphenols after transfection with the vectors according to
table 2.

| | Set | | | | | | | | |
|---|---|---|---|---|---|---|---|---|---|
| | fold change [set/control] | | | | | | | | |
| Compound | 1 | 2 | 3 | 4 | 5 | 6 | 7 | 8 | 9 |
| Quercetin 3-O-(6- | 919.82 | 143.92 | 1600.95 | 9.22 | 4.46 | 290.02 | 749.48 | 939.49 | 1313.19 |
| O-malonyl-beta-D- | | | | | | | | | |
| glucoside) | | | | | | | | | |
| Kaempferol-3-O- | 346.89 | 19.66 | 407.24 | 6.13 | 2.50 | 265.88 | 299.28 | 20.17 | 217.29 |
| galactoside | | | | | | | | | |
| Quercitrin/ | 593.97 | 85.04 | 471.22 | 2.61 | 2.76 | 378.61 | 440.03 | 47.80 | 486.12 |
| Kaempferol 3-O- | | | | | | | | | |
| glucoside | | | | | | | | | |
| Neohesperidin | 1.09 | 1.78 | 1.56 | 1.33 | 0.57 | 0.66 | 1.52 | 1.61 | 1.43 |
| Quercetin 3-O- | 401.01 | 74.21 | 339.77 | 5.91 | 2.26 | 181.95 | 364.34 | 244.67 | 374.70 |
| glucoside | | | | | | | | | |
| Myricitrin | 149.92 | 38.81 | 196.43 | 13.95 | 3.40 | 105.60 | 163.67 | 66.57 | 112.57 |
| Ramnazin-3-O- | 836.89 | 350.05 | 729.22 | 15.46 | 3.87 | 424.10 | 747.45 | 697.13 | 544.32 |
| rutinoside | | | | | | | | | |
| Myricitrin 2′-O- | 0.11 | 0.33 | 0.10 | 0.26 | 1.13 | 0.72 | 0.14 | | 0.31 |
| gallate | | | | | | | | | |
| Dihydrokaempferol | ↑ | ↑ | ↑ | | | | | ↑ | ↑ |
| Fustin | ↑ | ↑ | ↑ | | | | ↑ | ↑ | |
| Genistein 7-O- | ↑ | | ↑ | | | | ↑ | | ↑ |
| beta-D-glucoside | | | | | | | | | |
| Helichrysin A | | | | | | | | | |
| Kaempferol | ↑ | ↑ | ↑ | ↑ | | ↑ | ↑ | ↑ | ↑ |
| Naringenin/ | ↑ | | ↑ | | | | ↑ | | |
| Naringenin | | | | | | | | | |
| chalcone | | | | | | | | | |
| Neochanin | | | | | | | | | |
| Neoeriocitrin | ↑ | ↑ | ↑ | | | ↑ | ↑ | ↑ | ↑ |
| Ononin | ↑ | | ↑ | | | | ↑ | | ↑ |
| Pinocembrin | | | | | | | | | |
| Quercetin-3-methyl | ↑ | ↑ | ↑ | ↑ | | ↑ | ↑ | ↑ | ↑ |
| ether | | | | | | | | | |
| Salipurposid | ↑ | | ↑ | | | ↑ | ↑ | | ↑ |
| Scutellarein | ↑ | | ↑ | | | | ↑ | | ↑ |
| Sissotrin | | | | | | | | | |
| Taxifolin | | ↑ | ↑ | | | | | ↑ | ↑ |
| (dihydroquercetin) | | | | | | | | | |
| Vicenin-2 | ↑ | | ↑ | | | ↑ | ↑ | | ↑ |
| Total polyphenols | 240.23 | 66.89 | 228.92 | 5.44 | 2.04 | 131.05 | 211.53 | 140.35 | 185.57 |

↑—denotes that the polyphenol was detected in Nicotiana tabacum after transfection with the vector according to table 2, where the polyphenol wasn't present in the control. Blank cells mean that no expression was observed.

↑—denotes that the polyphenol was detected in Nicotiana tabacum after transfection with the vector according to table 2, where the polyphenol wasn't present in the control. Blank cells mean that no expression was observed.

TABLE 4
fold change in polyphenols (phenylpropanoid pathway) after
transfection with the vectors according to table 2.

| | Set | | | | | | | | |
|---|---|---|---|---|---|---|---|---|---|
| | fold change [set/control] | | | | | | | | |
| Compound | 1 | 2 | 3 | 4 | 5 | 6 | 7 | 8 | 9 |
| Neochlorogenic acid | 0.76 | 0.87 | 0.71 | 0.67 | 0.93 | 0.81 | 0.69 | 0.73 | 0.73 |
| Chlorogenic acid | 2.54 | 2.11 | 2.51 | 2.53 | 1.58 | 1.80 | 2.16 | 3.04 | 2.50 |
| Fraxin | 2.96 | 1.48 | 1.63 | 1.71 | 1.23 | 1.83 | 2.07 | 2.38 | 2.41 |
| 5-O-Caffeoylshikimic | 1.88 | 2.41 | 1.31 | 1.24 | 0.79 | 1.73 | 1.11 | 2.08 | 1.46 |
| acid | | | | | | | | | |
| Caffeic acid | 3.90 | 3.16 | 3.43 | 0.51 | 0.73 | 2.41 | 2.87 | 6.13 | 2.13 |
| 3,4-Dicaffeoylquinic | 7.58 | 3.36 | 3.31 | 1.36 | 1.33 | 2.84 | 6.16 | 4.99 | 4.49 |
| acid | | | | | | | | | |
| Scopoletin | 8.19 | 87.66 | 50.87 | 7.11 | 2.36 | 1.42 | 3.88 | 34.43 | 7.83 |
| Sinapoyl aldehyde | 1.32 | 1.45 | 1.17 | 1.44 | 2.22 | 1.74 | 1.47 | 1.36 | 1.34 |
| 5-Hydroxyferulic acid | 0.06 | 0.10 | | 0.08 | 0.92 | 0.26 | | | 0.07 |
| Aesculin \| Esculin | 6.79 | 6.35 | 6.55 | 2.52 | 2.90 | 4.30 | 6.67 | 4.34 | 5.02 |
| Coniferin | 32.96 | 43.19 | 30.45 | 2.90 | 1.93 | 17.67 | 26.98 | 9.23 | 20.12 |
| Ferulic acid | 13.08 | | | 2.14 | 1.30 | 3.47 | 6.02 | | 9.57 |
| Syringin | 3.21 | 5.61 | 4.69 | 1.15 | 2.04 | 7.65 | 4.17 | 1.77 | 3.52 |
| Fraxetin | 5.23 | 3.33 | 3.98 | 4.90 | 4.33 | 3.69 | 3.04 | 4.05 | 5.00 |
| 4-Hydroxy-3- | 1.40 | 1.40 | 1.83 | 0.78 | 0.81 | 1.13 | 1.37 | 1.16 | 1.63 |
| methoxycinnamaldehyde | | | | | | | | | |
| Esculetin | 2.24 | 4.64 | 5.35 | 2.92 | 1.49 | 1.50 | 3.33 | 4.84 | 3.91 |
| Umbelliferone | 1.44 | | | | | | 1.31 | | 1.39 |
| Coumarinic acid-beta- | ↑ | ↑ | ↑ | ↑ | ↑ | ↑ | ↑ | ↑ | ↑ |
| D-glucoside | | | | | | | | | |
| o-Coumaric acid | | | | | | | | | |
| p-Coumaric acid | ↑ | ↑ | ↑ | ↑ | | | ↑ | ↑ | ↑ |
| Phlorizin | ↑ | ↑ | ↑ | | | ↑ | ↑ | ↑ | ↑ |
| Rutarin | | | | | | | | | |
| Scoparone | ↑ | ↑ | ↑ | | ↑ | ↑ | ↑ | ↑ | ↑ |
| Sinapic acid | ↑ | ↑ | ↑ | | ↑ | | ↑ | | ↑ |
| Total polyphenols | 1.59 | 1.66 | 1.63 | 1.47 | 1.21 | 1.26 | 1.36 | 1.83 | 1.53 |

↑ - denotes that the polyphenol was detected in Nicotiana tabacum after transfection with the vector according to table 2, where the polyphenol wasn't present in the control. Blank cells mean that no expression was observed.

Polyphenols were detected using HPTLC, in freeze dried leaves of the transfected Nicotiana tabacum plants.

The different transcription factor combinations, listed in table 2, had variable effects on the expression of different polyphenols and the different transcription factor combinations were also able to promote expression of many polyphenols that are not usually expressed in native N. tabacum.

The overall level of polyphenol expression was promoted by all vectors of sets 1-9 for polyphenols that occur at all stages of the flavonoid pathway, including the phenylpropanoid pathway stage which precedes the flavonoid pathway.

It was also found that both quercetin and kaempferol could be generated by including an additional step or steps, in the preparation of the transfected N. tabacum leaves, of treating the leaves by acid hydrolysis or curing of the leaves before running the HPTLC analysis.

It is clear from the HPTLC results presented above and in FIGS. 2A and 2B that the expression of multiple different compounds are promoted in plants transfected with the vector components of the composition described herein. The quercetin standard (FIG. 2A) and kaempferol standard (FIG. 2B) confirm that the expression of both quercetin and kaempferol is promoted in leaves transfected according to set 3 and set 9 of the table above. The additional peaks seen in the HPTLC indicate that the expression of further compounds is also promoted, many of which are listed in tables 3 and 4.

The increased expression and/or generation of the listed polyphenols was a surprising finding, as it was expected that the transcription factors MYB12, DEL and ROS1, or AN2, TT8 and MYB12, would drive the generation of anthocyanin's. Instead, it was found that the pathway diverted towards the expression of quercetin, kaempferol and associated polyphenols.

### Example 2: Promoting the Expression of Polyphenols in Nicotiana benthamiana

Plasmids comprising combinations of SPS1, SPS2, AmDEL, AmROS1, and AtMYB12 were generated and used to transfect Nicotiana benthamiana. The combinations were as set out in table 5 below.

TABLE 5
Combinations of transcription factors in the vectors of sets 2-7.

| Set | Enzymes/Transcription Factors |
|---|---|
| 2 | SPS2, AtMYB12 |
| 3 | SPS2, AmDEL, AmROS1 |
| 4 | SPS2, AtMYB12, AmDEL, AmROS1 |
| 5 | SPS1, AtMYB12 |
| 6 | SPS1, AmDEL, AmROS1 |
| 7 | SPS1, AtMYB12, AmDEL, AmROS1 |
| 8 | control |

An Agrobacterium culture (Agrobacterium tumefaciens) was used to deliver the genetic material into the Nicotiana benthamiana cells. The plasmids containing the genes of interest were introduced into separate Agrobacterium cultures and cultured to a specific density.

Once the specific density was reached (OD600 of ~1-2), it was resuspended at a density of OD600 0.2 and prepared as an infiltration solution. Infiltration solutions were prepared for each of the plasmid types (see table 5).

The infiltration solutions were then individually infiltrated into the leaves of the Nicotiana benthamiana plants, using a syringe or vacuum infiltration. Separate plants were used for each set.

Following infiltration, the plants were incubated under controlled conditions (22 °C, ~250 μmol/m2/s (light), 70% humidity and 16 hr day/night cycle) in a growth chamber for 7 days. Following this, the infiltrated leaves were frozen instantly.

The dried leaves were then ground and extraction was preformed using methanol. Extracts were then filtered and placed in vials to be analysed by HPTLC. Stationary Phase; Merck, HPTLC Silica gel 60 F₂₅₄, Mobile phase; Chloroform:Methanol 9:1 (v/v), Chloroform:Methanol:Water 80:20:2 (v/v/v), Chloroform:Methanol:Water 61:32:5 (v/v/v), Toluene:Ethyl acetate:Acetic acid:Chloroform 5:5:1:2 (v/v/v/v). After separation, the plates were derivatized with 10% H2SO4 in methanol and then visualized by normal light or UV lamp.

TABLE 6
fold change in polyphenols after transfection
with the vectors according to table 5.

| | Set | | | | | |
|---|---|---|---|---|---|---|
| | fold change [set/control] | | | | | |
| Compound | 2 | 3 | 4 | 5 | 6 | 7 |
| Quercetin 3-O-(6- | 387.31 | 0.42 | 12.37 | 79.15 | 0.29 | 11.94 |
| O-malonyl-beta-D- | | | | | | |
| glucoside) | | | | | | |
| Kaempferol-3-O- | 63.96 | 0.69 | 19.61 | 60.55 | 0.47 | 28.99 |
| galactoside | | | | | | |
| Quercitrin/ | 731.60 | 0.96 | 15.10 | 157.08 | 1.00 | 19.21 |
| Kaempferol 3-O- | | | | | | |
| glucoside | | | | | | |
| Neohesperidin | | ↑ | ↑ | | ↑ | |
| Quercetin 3-O- | 1335.95 | 1.01 | 17.56 | 344.11 | 0.75 | 22.27 |
| glucoside | | | | | | |
| Myricitrin | 77.98 | 0.41 | 12.05 | 63.10 | 0.27 | 14.89 |
| Ramnazin-3-O- | 17.38 | 0.16 | 2.74 | 8.99 | 0.13 | 1.37 |
| rutinoside | | | | | | |
| Myricitrin 2′-O- | ↑ | | | ↑ | | |
| gallate | | | | | | |
| Dihydrokaempferol | ↑ | ↑ | ↑ | ↑ | | ↑ |
| Fustin | ↑ | ↑ | ↑ | ↑ | ↑ | ↑ |
| Genistein 7-O- | ↑ | ↑ | ↑ | ↑ | ↑ | ↑ |
| beta-D-glucoside | | | | | | |
| Helichrysin A | ↑ | ↑ | ↑ | ↑ | | ↑ |
| Kaempferol | ↑ | | ↑ | ↑ | | ↑ |
| Naringenin/ | ↑ | ↑ | ↑ | ↑ | | ↑ |
| Naringenin | | | | | | |
| chalcone | | | | | | |
| Neochanin | ↑ | ↑ | ↑ | ↑ | ↑ | ↑ |
| Neoeriocitrin | ↑ | ↑ | ↑ | ↑ | ↑ | ↑ |
| Ononin | ↑ | ↑ | ↑ | ↑ | ↑ | ↑ |
| Pinocembrin | ↑ | ↑ | | ↑ | ↑ | ↑ |
| Quercetin-3- | ↑ | | | | | |
| methyl ether | | | | | | |
| Salipurposid | ↑ | ↑ | ↑ | ↑ | ↑ | ↑ |
| Scutellarein | ↑ | ↑ | ↑ | ↑ | | ↑ |
| Sissotrin | ↑ | ↑ | ↑ | ↑ | ↑ | ↑ |
| Taxifolin | ↑ | ↑ | ↑ | ↑ | | ↑ |
| (dihydroquercetin) | | | | | | |
| Vicenin-2 | ↑ | ↑ | ↑ | ↑ | | ↑ |
| Total | 413.77 | 6.18 | 49.94 | 132.68 | 1.57 | 28.76 |
| polyphenols | | | | | | |

↑ - denotes that the polyphenol was detected in Nicotiana benthamiana after transfection with the vector according to table 5, where the polyphenol wasn't present in the control. Blank cells mean that no expression was observed.

TABLE 7
fold change in polyphenols (phenylpropanoid pathway) after
transfection with the vectors according to table 5.

| | Set | | | | | |
|---|---|---|---|---|---|---|
| | −fold increase [(set-control)/control] | | | | | |
| Compound | 2 | 3 | 4 | 5 | 6 | 7 |
| Neochlorogenic acid | 1.15 | 0.84 | 0.63 | 0.97 | 1.16 | 0.69 |
| Chlorogenic acid | 1.17 | 0.76 | 0.63 | 1.10 | 1.24 | 0.72 |
| Fraxin | 2.74 | 5.34 | 2.31 | 3.73 | 9.01 | 2.34 |
| 5-O-Caffeoylshikimic | 0.21 | 1.24 | 0.15 | 0.41 | 8.26 | 0.92 |
| acid | | | | | | |
| Caffeic acid | 0.47 | 0.55 | 0.20 | 0.40 | 0.41 | 0.26 |
| 3,4-Dicaffeoylquinic acid | 13.81 | 2.03 | 1.22 | 8.14 | 3.96 | 4.62 |
| Scopoletin | | | | | | |
| Sinapoyl aldehyde | 1.71 | 1.87 | 1.28 | 0.83 | 1.01 | 0.79 |
| 5-Hydroxyferulic acid | 1.15 | 1.41 | 1.54 | 1.06 | 1.69 | 1.26 |
| Aesculin \| Esculin | ↑ | ↑ | ↑ | ↑ | ↑ | ↑ |
| Coniferin | 6.04 | 2.57 | 1.85 | 29.57 | 69.72 | 32.24 |
| Ferulic acid | ↑ | ↑ | ↑ | ↑ | | ↑ |
| Syringin | 0.49 | 0.42 | 0.38 | 0.30 | 0.31 | 0.23 |
| Fraxetin | | | ↑ | | | |
| 4-Hydroxy-3- | 0.43 | 0.55 | 0.45 | 0.54 | | 0.34 |
| methoxycinnamaldehyde | | | | | | |
| Esculetin | 0.27 | 0.38 | | 0.16 | 0.31 | 0.28 |
| Umbelliferone | | ↑ | ↑ | ↑ | ↑ | ↑ |
| Coumarinic acid-beta-D- | | ↑ | ↑ | | | |
| glucoside | | | | | | |
| o-Coumaric acid | ↑ | ↑ | | ↑ | ↑ | ↑ |
| p-Coumaric acid | 3.82 | 12.21 | 3.33 | 1.32 | 1.70 | 15.02 |
| Phlorizin | ↑ | ↑ | ↑ | ↑ | ↑ | ↑ |
| Rutarin | ↑ | ↑ | ↑ | | ↑ | ↑ |
| Scoparone | | ↑ | | | | |
| Sinapic acid | 2.15 | 3.94 | 2.71 | 1.86 | 2.50 | 1.77 |
| Total polyphenols | 1.18 | 0.79 | 0.63 | 1.06 | 1.23 | 0.72 |

↑ - denotes that the polyphenol was detected in Nicotiana benthamiana after transfection with the vector according to table 5, where the polyphenol wasn't present in the control. Blank cells mean that no expression was observed.

Polyphenols were detected using HPTLC, in freeze dried leaves of the transfected Nicotiana benthamiana plants.

The different transcription factor combinations, listed in table 5, had variable effects on the expression of different polyphenols and the different transcription factor combinations were also able to promote expression of many polyphenols that are not usually expressed in native N. benthamiana. The overall level of polyphenol expression was promoted by all vectors of sets 2-7 for the polyphenols that occur after the phenylpropanoid stage of the flavonoid pathway and by the vectors of sets 2, 5 and 6 for polyphenols of the phenylpropanoid pathway.

It was also found that both quercetin and kaempferol could be generated by including an additional step or steps, in the preparation of the transfected N. tabacum leaves, of treating the leaves by acid hydrolysis or curing of the leaves before running the HPTLC analysis.

It is clear from the HPTLC results presented above that the expression of multiple different compounds are promoted in plants transfected with the vector components of the composition described herein.

The increased expression and/or generation of the listed polyphenols was a surprising finding, as it was expected that the transcription factors MYB12, DEL and ROS1, would drive the generation of anthocyanin's. Instead, it was found that the pathway diverted towards the expression of quercetin, kaempferol and associated polyphenols.

### Example 3: Promoting the Expression of Catechin in Nicotiana tabacum and Nicotiana benthamiana

Plasmids comprising combinations of ANR, LAR, DEL, ROS, MYB330, MYB12, MYB5a and AN2 were generated and used to transfect Nicotiana tabacum and Nicotiana benthamiana.

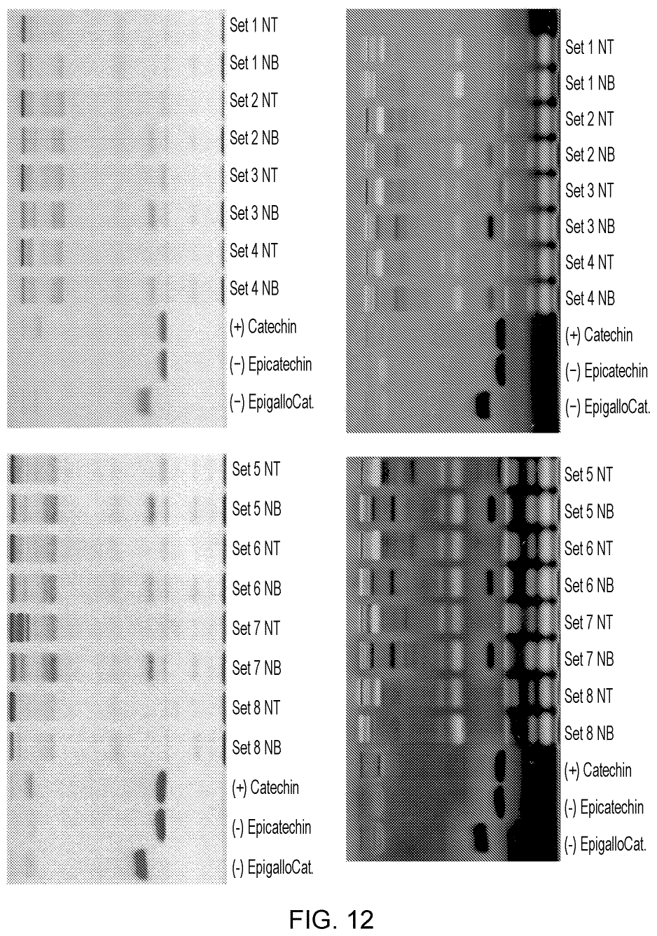

The combinations were as set out in table 8 below, which corresponds to the HPTLC results in FIG. 12.

TABLE 8
Combinations of transcription factors
and enzymes in the vectors of sets 1-8.

| Set | Transcription Factors |
|---|---|
| 1 | ANR, LAR |
| 2 | ANR, LAR, DEL, ROS |
| 3 | ANR, LAR, DEL, ROS, MYB330 |
| 4 | ANR, LAR, DEL, ROS, MYB12 |
| 5 | ANR, LAR, DEL, ROS, MYB5a |
| 6 | ANR, LAR, DEL, ROS, AN2 |
| 7 | ANR, LAR, DEL, ROS, MYB330, MYB12, MYB5a, AN2 |
| 8 | Control GFP |

An Agrobacterium culture (Agrobacterium tumefaciens) was used to deliver the genetic material into the Nicotiana tabacum and Nicotiana benthamiana cells. The plasmids containing the genes of interest were introduced into separate Agrobacterium cultures and cultured overnight to reach specific density.

Once the specific density was reached (OD600 of ~1-2), it was resuspended at a density of OD600 0.2 and prepared as an infiltration solution. Infiltration solutions were prepared for each of the plasmid types (see table 8).

The infiltration solutions were then individually infiltrated into the leaves of the Nicotiana tabacum and Nicotiana benthamiana plants, using a syringe or vacuum infiltration. Separate plants were used for each set of transcription factors.

Following infiltration, the plants were incubated under controlled conditions (22 °C, ~250 μmol/m2/s (light), 70% humidity and 16 hr day/night cycle) in a growth chamber for 7 days. Following this, the infiltrated leaves were removed from the plant and frozen instantly.

The dried leaves were then ground and extraction was preformed using 100% methanol. Extracts were then filtered and placed in vials to be analysed by HPTLC. Stationary Phase; Merck, HPTLC Silica gel 60 F₂₅₄, glass plates 20×10 cm, Mobile phase; Cyclopentyl Methyl Ether, Tetrahydrofuran, Water, Formic acid 40:24:1:1 (V/V). After separation, the plates were derivatized with 10% H₂SO₄ in methanol and then visualized by normal light or UV lamp.

It is clear from the HPTLC results presented in FIG. 12 that the various combinations of enzymes and transcription factors listed in table 8 promote the expression of various compounds compared to the GFP control. In particular, the expression of catechin is promoted using the combinations defined by sets 2, 3, 5, 6, and 7] compared to the control, with set 5 resulting in the greatest expression.

### Example 4: Promoting the Expression of Apigenin-7-O-Glucoside in Nicotiana tabacum and Nicotiana benthamiana

Plasmids comprising combinations of AtMYB12, FNSI, UGT84A23, UGT84A24, DEL, and ROS, were generated and used to transfect Nicotiana tabacum and Nicotiana benthamiana.

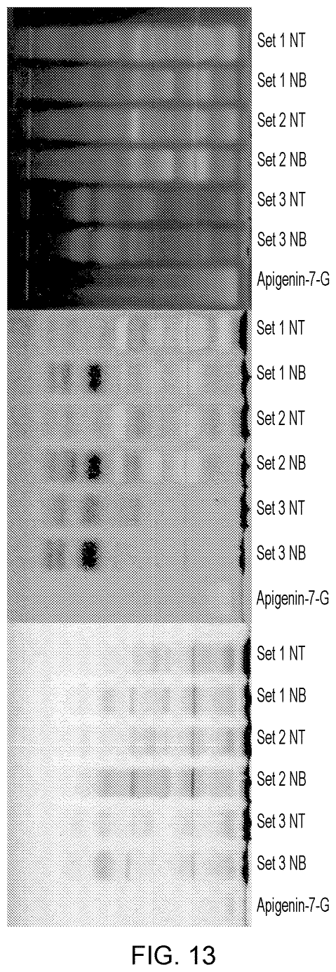

The combinations were as set out in table 9 below, which corresponds to the HPTLC results in FIG. 13.

TABLE 9
Combinations of transcription factors
and enzymes in the vectors of sets 1-3.

| Set | Transcription Factors |
|---|---|
| 1 | AtMYB12, FNSI, UGT84A23, UGT84A24 |
| 2 | AtMYB12, DEL, ROS, FNSI, UGT84A23, UGT84A24 |
| 3 | Control GFP |

An Agrobacterium culture (Agrobacterium tumefaciens) was used to deliver the genetic material into the Nicotiana tabacum and Nicotiana benthamiana cells. The plasmids containing the genes of interest were introduced into separate Agrobacterium cultures and cultured overnight to reach specific density.

Once the specific density was reached (OD600 of ~1-2), it was resuspended at a density of OD600 0.2 and prepared as an infiltration solution. Infiltration solutions were prepared for each of the plasmid types (see table 9).

The infiltration solutions were then individually infiltrated into the leaves of the Nicotiana tabacum and Nicotiana benthamiana plants, using a syringe or vacuum infiltration. Separate plants were used for each set of transcription factors.

Following infiltration, the plants were incubated under controlled conditions (22 °C, ~250 μmol/m2/s (light), 70% humidity and 16 hr day/night cycle) in a growth chamber for 7 days. Following this, the infiltrated leaves were removed from the plant and frozen instantly.

The dried leaves were then ground and extraction was preformed using 100% methanol. Extracts were then filtered and placed in vials to be analysed by HPTLC. Stationary Phase; Merck, HPTLC Silica gel 60 F₂₅₄, glass plates 20×10 cm, Mobile phase; Ethanol, Dichloromethne, Water, Formic acid 16:16:4:1 (V/V). After separation, the plates were derivatized with 10% H₂SO₄ in methanol and then visualized by normal light or UV lamp.

It is clear from the HPTLC results presented in FIG. 13 that the combinations of enzymes and transcription factors listed in table 9 promote the expression of various compounds compared to the GFP control. In particular, the expression of apigenin-7-O-glucoside is promoted using the combinations defined by sets 1 and 2 compared to the control, with set 1 resulting in the greatest expression.

### Example 4: Promoting the Expression of Epigallocatechin in Nicotiana tabacum and Nicotiana benthamiana

Plasmids comprising combinations of ANR, LAR, DEL, ROS, MYB330, MYB12, MYB5a and AN2 were generated and used to transfect Nicotiana tabacum and Nicotiana benthamiana.

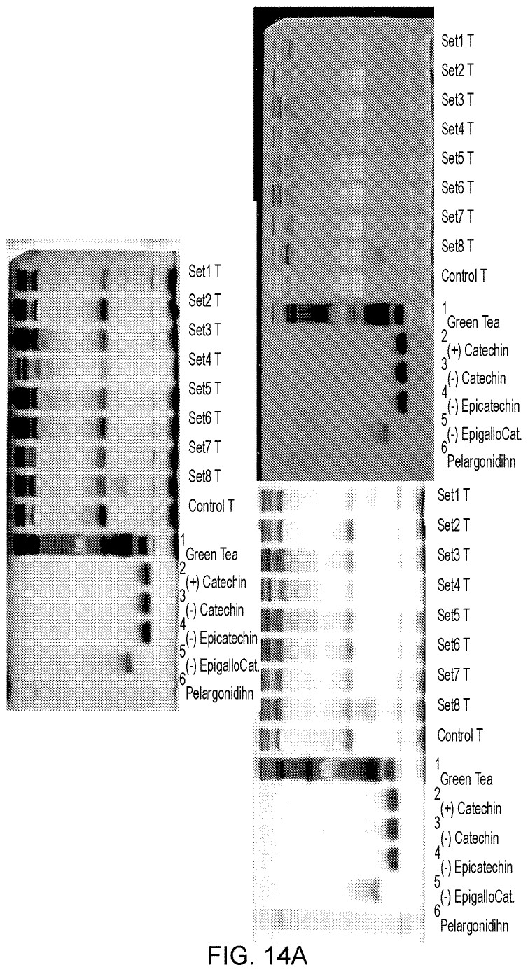

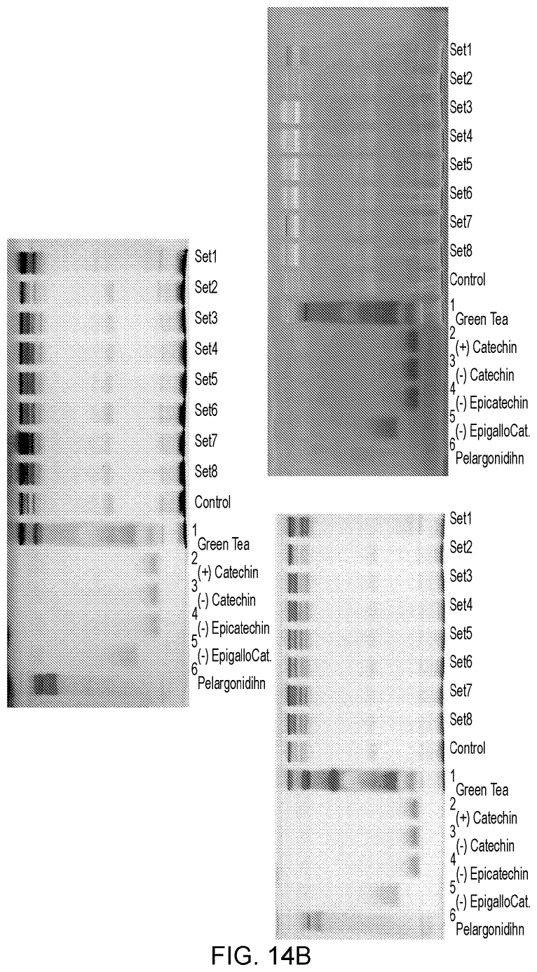

The combinations were as set out in table 10 below, which corresponds to the HPTLC results in FIGS. 14A (Nicotiana tabacum) and 14B (Nicotiana benthamiana).

TABLE 10
Combinations of transcription factors and enzymes
in the vectors of sets 1-8.

| Set | Transcription Factors |
|---|---|
| 1 | ANR, LAR, DEL, ROS |
| 2 | MYB330 |
| 3 | MYB330, MYB12 |
| 4 | MYB330, MYB12, MYB5a |
| 5 | MYB330, MYB12, MYB5a, AN2 |
| 6 | ANR, LAR, MYB330, MYB12, MYB5a, AN2 |
| 7 | DEL, ROS, MYB330, MYB12, MYB5a, AN2 |
| 8 | ANR, LAR, DEL, ROS, MYB330, MYB12, MYB5a, AN2 |

An Agrobacterium culture (Agrobacterium tumefaciens) was used to deliver the genetic material into the Nicotiana tabacum and Nicotiana benthamiana cells. The plasmids containing the genes of interest were introduced into separate Agrobacterium cultures and cultured overnight to reach specific density.

Once the specific density was reached (OD600 of ~1-2), it was resuspended at a density of OD600 0.2 and prepared as an infiltration solution. Infiltration solutions were prepared for each of the plasmid types (see table 10).

The infiltration solutions were then individually infiltrated into the leaves of the Nicotiana tabacum and Nicotiana benthamiana plants, using a syringe or vacuum infiltration. Separate plants were used for each set of transcription factors.

Following infiltration, the plants were incubated under controlled conditions (22 °C, ~250 μmol/m2/s (light), 70% humidity and 16 hr day/night cycle) in a growth chamber for 7 days. Following this, the infiltrated leaves were removed from the plant and frozen instantly.

The dried leaves were then ground and extraction was preformed using 100% methanol. Extracts were then filtered and placed in vials to be analysed by HPTLC. Stationary Phase; Merck, HPTLC Silica gel 60 F₂₅₄, glass plates 20×10 cm, Mobile phase; Cyclopentyl Methyl Ether, Tetrahydrofuran, Water, Formic acid 40:24:1:1 (V/V). After separation, the plates were derivatized with 10% H₂SO₄ in methanol and then visualized by normal light or UV lamp.

It is clear from the HPTLC results presented in FIGS. 14A and 14B that the various combinations of enzymes and transcription factors listed in table 10 promote the expression of various compounds compared to the GFP control. In particular, the expression of epigallocatechin is promoted using the combinations defined by sets 1 and 8 compared to the control, with set 8 resulting in the greatest expression.

1. A composition for promoting the expression of one or more polyphenols in a cell, wherein the composition comprises:
a) one or more first vector components encoding one or more transcription factors present in the flavonoid pathway; and optionally
b) one or more second vector components encoding one or more enzymes present in the flavonoid pathway.

2. The composition according to claim 1, wherein the one or more first vector components are provided in a first vector and the one or more second vector components are provided in a second vector.

3. The composition according to claim 1, wherein the one or more first vector components and the one or more second vector components are provided in a single vector.

4. The composition according to claim 1, wherein the one or more polyphenols comprise one or more flavonoids, and/or wherein the one or more flavonoids comprise one or more plant-derived flavonoids.

5. (canceled)

6. The composition according to claim 1, wherein the one or more transcription factors present in the flavonoid pathway comprise myeloblastosis (MYB) type transcription factors and/or basic helix-loop-helix (bHLH) type transcription factors, and/or wherein the one or more transcription factors present in the flavonoid pathway comprise one or more of MYB, PAP1, TT2, AN2, TT8, TT9, DEL and ROS1.

7. (canceled)

8. The composition according to claim 6, wherein MYB is one or more of MYB12, MYB330, MYB14, MYBPA1, MYB5a, MYB5b, MYB75.

9. The composition according to claim 1, wherein the one or more transcription factors present in the flavonoid pathway are independently derived from a plant species, and/or wherein the plant species is selected from the group comprising At (Arabidopsis thaliana), Am (Antirrhinum majus), Nt (Nicotiana tabacum), Mt (Medicago truncatula), Vv (Vitis vini fera), or Cs (Camellia sinensis).

10. (canceled)

11. A plant, plant part or plant cell transformed with the vector components according to the composition of claim 1.

12. The plant, plant part or plant cell according to claim 11, wherein the plant, plant part or plant cell is algae, moss or any plant, plant part or plant cell derived from the genus Nicotiana.

13. The plant, plant part or plant cell according to claim 12, wherein the plant, plant part or plant cell derived from the genus Nicotiana is selected from group comprising Nicotiana tabacum, Nicotiana benthamiana, or BY-2 cells.

14. (canceled)

15. (canceled)

16. (canceled)

17. (canceled)

18. (canceled)

19. (canceled)

20. A method of promoting the expression of one or more polyphenols in a cell, comprising the step of:
a) introducing the composition according to claim 1 into a cell.

21. The method of claim 20, wherein the method further comprises:
b) extracting the one or more polyphenols from a cell to produce an extract;
c) detecting the one or more polyphenols in the extract; and
d) purifying the one or more polyphenols from the extract.

22. The method according to claim 21, wherein the cell is part of a plant, the method further comprises:
between step (a) and step (b), harvesting one or more leaves from the plant and treating the one or more leaves by curing and/or hydrolysis.

23. The method according to claim 21, wherein the one or more polyphenols are purified using Flash and preparative MPLC, HPTLC or LC/MS.

24. The method according to claim 20, wherein the cell is a plant cell.

25. The method according to claim 24, wherein the plant cell is an algae cell, moss cell, or any cell or cell line derived from the genus Nicotiana.

26. The method according to claim 25, wherein the cell or cell line derived from the genus Nicotiana is selected from the group comprising Nicotiana tabacum, Nicotiana benthamiana, or BY-2 cells.

27. The method according to claim 20, wherein the one or more polyphenols comprise one or more flavonoids, and/or wherein the one or more flavonoids comprise one or more plant-derived flavonoids.

28. (canceled)

29. A polyphenol obtainable from the method according to claim 20.

30. The polyphenol according to claim 29, wherein the polyphenol is a flavonoid, and/or wherein the flavonoid is a plant-derived flavonoid.

31. (canceled)


SEQUENCE LISTING (8 sequences)

SEQ ID NO: 1 - DNA, 14107 bp, synthetic construct
  source 1..14107: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 2 - DNA, 11571 bp, synthetic construct
  source 1..11571: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgggaagagcgccatgttgcgagaaggtcggtatcaagagagggcggtggacggcggaggaggaccagattctctccaactacattcaatccaacggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatgacgaacgcttcttcagcgccaccgccgccgcaggcaaaacgcagacttgggagaacgggtaggtccgctatgaaaccaaaaatccacagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtgatggggagttgcataacataagagactctgaaggagccagagggttctcggatacttggaaccaagggaatctcgactgtcttcttcagtcttgtccatcggtggagtcgtttctcaactacgaccaccaagttaacgatgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctgggccccccctcgaagtcgacggtattgataagcttgatatcgaattcctgcagcccgggggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 3 - DNA, 17068 bp, synthetic construct
  source 1..17068: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgggaagagcgccatgttgcgagaaggtcggtatcaagagagggcggtggacggcggaggaggaccagattctctccaactacattcaatccaacggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatgacgaacgcttcttcagcgccaccgccgccgcaggcaaaacgcagacttgggagaacgggtaggtccgctatgaaaccaaaaatccacagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtgatggggagttgcataacataagagactctgaaggagccagagggttctcggatacttggaaccaagggaatctcgactgtcttcttcagtcttgtccatcggtggagtcgtttctcaactacgaccaccaagttaacgatgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 4 - DNA, 10995 bp, synthetic construct
  source 1..10995: mol_type = other DNA
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgccagtgccaagctaattcgaggggatttaaattggtcgatctggttgtactgaggacttctacaagaaggacagctctgtttgaggtcggattgggatcgcttgaaaagcttgttctgttacgtttgatccctccaccatgagcaacgagctgaattgaaactcagaaaggtcttgctgcatcaactgatgcaggtggcaaagacactgaagggtcgatgaggtgcagagcgaatcgacagcagctgtaagggttcctcttccttctcctcctcctcctcctcttccttcgcagcaccgcattatgcttgaattgtgttgcgctccggcgtcactcccatgaacatgaaaacacggaccagcaccgaacctacgttttcatcgacgatttagtgttgaaacctcacctataatgacttcaatggctttgcaagtgcattagtagtgattacatacaataattttaaatctcaattggcactaaataatgtcaacaagattaaattactaccaaatccagttgggttacaaaatgaaacaaaagcatctgacaatctcaaacatgaaaagactcgtgcacatggtctcaaaaaagttagaaggtaatgattgagcaagaaaatactttatcacattcattgatgattgagtggaatctgaagggagatagcataaaggtcttacataacatttattgataattgagtacaattgaagtaaagcctggacaacaacttcaatacatctccaccgcggtggcggccgctctagaactagtggatcccccgggctgcaggaattagcttatcgataccgtcgacctcgagggggggcccggtaccaagcttgctagcgaattcgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctgaattcgatatcccgggcgcgcggtaccataacttcgtataatgtatgctatacgaagttatcctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcatggagaaccgacctgcaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcagggactcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggacggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccataacttcgtataatgtatgctatacgaagttatgcatgcgatagcagctgtccttctcaagtggaaaggtagtgtgaataggtggagtgagggaagagcttctcctcaagtgctctgacaactttagggtttatttgcaggaagctaccaagtgttgaatgaccaagccatcgctttatgattgcaagagtactccctgacacgcgtggaacaggggattaagtgaatgggagggaatgcgtttctgagaggaatctcatgttctactatgagaatgtagagaaaacaagaggcgtccgtgttgcacttgtgtgggtgcatgtcgtacttgcaagttttgtagaggaaaacttaagttacttatgtttcgcaagagttttaggtttcgagggatcgccactgccagatttagacacacacttttcaaagagtgagagggggcgaggttgaggggagaacgctggcgcgcacagggggcgcctgatggctgatcggcctcaagcacatgtaacttttcatgaaattcgtctggaagtgaaagagccgaagtacaaaagcgtgagtatcatcttaagcggagctggagataagggagggggcgaaagcggggaagaatgctgagtgtagtgcgggtgcacctcccgaccgacattcccattgtaggctgcgagttgtcggcctatgtgctgttgcgacggggagattcgagtctttctcctgaagatgttaccgaggcatcgtctacggatggctattttctccagtgtcgatggtatctgtccttacttctaaacttttctttttgtttattgtggaacattcatgctaacgttcttcttgaaagacgacagtccgtgagttgaatttaaatcccatctcgagatctagcttggc

SEQ ID NO: 5 - DNA, 5340 bp, synthetic construct
  source 1..5340: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggatcccacacacctgcccgtctgcctgacaggaagtgaacgcatgtcgagggaggcctcaccaatcgtcacacgagccctcgtcagaaacacgtctccgccacgctctccctctcacggccgaccccgcagcccttttgccctttcctaggccaccgacaggacccaggcgctctcagcatgcctcaacaacccgtactcgtgccagcggtgcccttgtgctggtgatcgcttggaagcgcatgcgaagacgaaggggcggagcaggcggcctggctgttcgaagggctcgccgccagttcgggtgcctttctccacgcgcgcctccacacctaccgatgcgtgaaggcaggcaaatgctcatgtttgcccgaactcggagtccttaaaaagccgcttcttgtcgtcgttccgagacatgttagcagatcgcagtgccacctttcctgacgcgctcggccccatattcggacgcaattgtcatttgtagcacaattggagcaaatctggcgaggcagtaggcttttaagttgcaaggcgagagagcaaagtgggacgcggcgtgattattggtatttacgcgacggcccggcgcgttagcggcccttcccccaggccagggacgattatgtatcaatattgttgcgttcgggcactcgtgcgagggctcctgcgggctggggagggggatctgggaattggaggtacgaccgagatggcttgctcggggggaggtttcctcgccgagcaagccagggttaggtgttgcgctcttgactcgttgtgcattctaggaccccactgctactcacaacaagccaaaatgcatggccgcgctccctgctgcgagaaggtgggcatcaagcgcggccgctggaccgcggaggaggaccagatcctgagcaactacatccagtcgaacggcgagggctcctggcgcagcctgccaaagaacgccggcctgaagcgctgcggcaagtcgtgccgcctgcgctggatcaactacctgcgctccgacctgaagcgcggcaacattacccccgaggaggaggagctggtggtcaagctgcacagcaccctgggcaaccgctggagcctgatcgccggccacctgcccggccgcaccgacaacgagatcaagaactactggaacagccacctgtcgcgcaagctgcacaacttcatccgcaagccctccatcagccaggacgtgagcgctgtgatcatgaccaacgcctcgagcgcgcctcccccgccccaggccaagcgccgcctgggccgcaccggccgctcggccatgaagcccaagatccaccgcaccaagacccgcaagaccaagaagaccagcgcgccgccggagcccaacgccgacgtggccggcgccgacaaggaggcgctgatggtggagagctcgggcgctgaggcggagctgggccgcccctgcgactactacggcgacgactgcaacaagaacctgatgagcatcaacggcgacaacggcgtgctgacgttcgacgacgacatcatcgacctgctgctggacgagagcgaccccggccacctgtacacgaacaccacctgcggcggcgacggcgagctgcacaacatccgcgacagcgagggcgcccgcggcttcagcgacacgtggaaccagggcaacctggactgcctgctgcagagctgcccctccgtggagagcttcctgaactacgaccaccaggtgaacgacgctagcacggacgagttcatcgactgggactgcgtgtggcaggagggcagcgacaacaacctgtggcacgagaaggagaaccccgacagcatggtgagctggctgctggacggcgacgacgaggcaacgatcggcaacagcaactgcgagaacttcggcgagcccctggaccacgacgacgagagcgccctggtggcctggctgctgagctaaccggtgcgctttcgccatctgcgggggtcgtaggctagaactggggttggggatcgggctgcttgcatagccaagcaattttccatctggccagcaatggcctagcactatgagcggttcaagtgtctcttgtgtgttgtgtcgcattgcatggccgtggtgacctgcaattttctgtaaccggacatgcagaagcttcggttcgcgtcctttcctcgcttgctacgggatcgggagtcggcagggctagaagacttgggtaacacgcgcaattcagcaatacaggccagccagggcagcgaagggggacttcagcaaggacctctcgggaatagtggaagagctaggaggggtacagcagtagagatcgaggggtccagctcacagttctattacgtcgttcgtggggacgaactgggtcgaggcgctacggactcgaaatgacggcagagggtggcaaggaaggggagcaccactgtgagcagttgcagcggcacatacactacgtctcttggccttaagcacagccagcacacttgtacggggcaacagtagccccgaagcagcctgatgcagtcacaccgtgccgggccagtgttaacaaggaagggcaggcaccagggcgagggcaggcgcggcaaaactcgccggttcctgacacggtgacacgcaggtatacggtgacagctcagctagtgataccagctgctccgcgttctgaggagagatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 6 - DNA, 8635 bp, synthetic construct
  source 1..8635: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggatcccacacacctgcccgtctgcctgacaggaagtgaacgcatgtcgagggaggcctcaccaatcgtcacacgagccctcgtcagaaacacgtctccgccacgctctccctctcacggccgaccccgcagcccttttgccctttcctaggccaccgacaggacccaggcgctctcagcatgcctcaacaacccgtactcgtgccagcggtgcccttgtgctggtgatcgcttggaagcgcatgcgaagacgaaggggcggagcaggcggcctggctgttcgaagggctcgccgccagttcgggtgcctttctccacgcgcgcctccacacctaccgatgcgtgaaggcaggcaaatgctcatgtttgcccgaactcggagtccttaaaaagccgcttcttgtcgtcgttccgagacatgttagcagatcgcagtgccacctttcctgacgcgctcggccccatattcggacgcaattgtcatttgtagcacaattggagcaaatctggcgaggcagtaggcttttaagttgcaaggcgagagagcaaagtgggacgcggcgtgattattggtatttacgcgacggcccggcgcgttagcggcccttcccccaggccagggacgattatgtatcaatattgttgcgttcgggcactcgtgcgagggctcctgcgggctggggagggggatctgggaattggaggtacgaccgagatggcttgctcggggggaggtttcctcgccgagcaagccagggttaggtgttgcgctcttgactcgttgtgcattctaggaccccactgctactcacaacaagccaaaatgcatgccaccggcatccagaaccagaagatcgtgcccgagaacctgcgcaagcagctggccattgcggtgcgctctatccagtggtcgtacgcgatcttctggagcaacagcgtggcgcagccgggagtgctggagtggggcgacggcttctacaacggcgacatcaagacgcgcaagaccgtgcagagcgtggagctgaaccaggaccagctgggcctgcagcgcagcgaccagctgcgcgagctgtacgagagcctgagcctgggcgagaccaacacgcaggcgaagcgccccaccgcggccctgagccccgaggacctgaccgacgccgagtggttcttcctggtgtgcatgagcttcatcttcaacatcggccagggcctgccgggccgcaccctggcccgcaaccaggctgtgtggctgtgcaacgcccaccgcgccgacaccaaggtgttctcccgcagcctgctggccaagtccgcgtccatccagaccgtggtgtgcttcccctacagcgagggcgtggtcgagctgggcgccacggagctggtgcccgaggacctgaacctgatccagcacatcaagacctcgttcctggacagccccgccaccgtgcccaagatcccgaactacgtgagcaacagcatcaccaataacaatgacctgatctgcgaggcgctggagcacgccaacatccccgagaacgacctggaccagctgctgaactgccccgacaccaacatctgctcccccgacaactctctggacgacttcgccgacaacctgctgatcgacgagagcaacctggcggagggcatcaacggggaggtgccccagacgcagagctggcccttcatggacgacgccatcagcaactgtctgaacagcagcatgaacagcagcgactgcatcagccagacccacgagaacctggagagcttcgcgccgctgagcgacggcaagggcccccccgagacgaacaactgcatgcacagcacccagaagtgcaaccagcagatcgagaacaccggcgtgcagggcgacgaggtgcactaccagggcgtgctgagcaacctgctgaagtcgtcccaccagctggtgctgggcccgtacttccgcaacggcaaccgcgagagctcgttcgtgagctggaacaaggacggcagctcgggcacccacgtgccgcgctcgggcacctcccagcgcttcctgaagaaggtgctgttcgaggtggcccgcatgcacgagaactcgcgcctggacgcgggcaagcagaagggcaacagcgactgcctcgccaagcccaccgctgacgagatcgaccgcaaccacgtgctgtccgagcgcaagcgccgcgagaagatcaacgagcgcttcatgatcctggcgagcctggtccccagcggcggcaaggtggacaaggtgagcatcctggaccacaccattgactacctgcgcggcctggagcgcaaggtggacgagctggagagcaacaagatggtgaagggccgcggccgcgagagcaccaccaagaccaagctgcacgacgccatcgagcgcacgagcgacaactacggcgcgacccgcacctccaacgtgaagaagcccctgaccaacaagcgcaaggcctccgacaccgacaagatcggcgcggtgaacagccgcggccgcctgaaggactcgctgacggacaacatcaccgtgaacatcaccaacaaggacgtgctgatcgtggtgacatgcagcagcaaggagttcgtcctgctggaggtgatggaggcggtgcgccgcctgtccctggacagcgagaccgtgcagagctccaaccgggacggcatgatcagcatcaccatcaaggctaagtgcaagggcctgaaggtggcctccgccagcgtgatcaagcaggcgctgcagaaggtgaccatgaagagcctgctggcgatccaccccaccgaggcgcgccacaagcagaagattgtggcccccgtgaagcagaccctgaacttcgacctgctgaagctggcgggcgacgtggagtccaacccgggccccatggagaagaactgccgcggcgtgcgcaagggcacctggaccaaggaggaggacaccctgctgcgccagtgcatcgaggagtacggcgagggcaagtggcaccaggtgccccaccgcgccggcctgaaccgctgccgcaagtcctgccgcctgcgctggctgaactacctgcgcccgaacatcaagcgcggccgcttcagccgcgacgaggtggacctgatcgtgcgcctgcacaagctgctgggcaacaagtggagcctgatcgccggccgcatccccggccgcaccgcgaacgacgtgaagaacttctggaacacccacgtgggcaagaacctgggcgaggacggcgagcgctgccgcaagaacgtgatgaacaccaagaccattaagctgaccaacatcgtgcgcccgcgcgcgcgcaccttcaccggcctgcacgtgacctggccccgcgaggtgggcaagacggacgagttctccaacgtgcgcctgaccacggacgagatccccgactgcgagaagcagacgcagttctacaacgacgtggcgtccccgcaggacgaggtggaggactgcatccagtggtggtcgaagctgctggagaccaccgaggacggcgagctgggcaacctgttcgaggaggcgcagcagatcggcaactaaccggtgcgctttcgccatctgcgggggtcgtaggctagaactggggttggggatcgggctgcttgcatagccaagcaattttccatctggccagcaatggcctagcactatgagcggttcaagtgtctcttgtgtgttgtgtcgcattgcatggccgtggtgacctgcaattttctgtaaccggacatgcagaagcttcggttcgcgtcctttcctcgcttgctacgggatcgggagtcggcagggctagaagacttgggtaacacgcgcaattcagcaatacaggccagccagggcagcgaagggggacttcagcaaggacctctcgggaatagtggaagagctaggaggggtacagcagtagagatcgaggggtccagctcacagttctattacgtcgttcgtggggacgaactgggtcgaggcgctacggactcgaaatgacggcagagggtggcaaggaaggggagcaccactgtgagcagttgcagcggcacatacactacgtctcttggccttaagcacagccagcacacttgtacggggcaacagtagccccgaagcagcctgatgcagtcacaccgtgccgggccagtgttaacaaggaagggcaggcaccagggcgagggcaggcgcggcaaaactcgccggttcctgacacggtgacacgcaggtatacggtgacagctcagctagtgataccagctgctccgcgttctgaggagagtcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacatgacacaagaatccctgttacttctcgaccgtattgattcggatgattcctacgcgagcctgcggaacgaccaggaattctgggaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggtgccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggccggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgataaggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 7 - DNA, 11118 bp, synthetic construct
  source 1..11118: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgaatatttgtactaataagtcgtcgtcaggagtgaagaaaggtgcatggactgaagaagaagatgttctattgaaaaaatgcatcgagaaatatggagaaggaaagtggcatcaagttcctcttagagctggtttgaatagatgcagaaagagctgcagattaaggtggctaaattatctaaggccacatataaagagaggagacttctcttttgatgaagtagatctcattttgaggcttcataagctgttaggcaacagatggtcacttattgctggtagacttcctggaaggacggcaaacgatgtcaaaaactactggaacagccatcttcgcaagaagttaattgctcctcatgatcaaaaggagagcaagcaaaaagcaaagaagatcaccatattcagacctcggcctcgaaccttctcaaagacaaatacttgtgttaaaagtaacacaaatactgtagataaggatattgaaggcagcagcgaaataattagattcaacgataatttgaagccaacaactgaagaattgacggatgatggaattcaatggtgggccgatttactagctaacaattacaacaataatgggattgaggaagctgataattcatcaccaactttgttgcatgaggaaatgccacttctcagttgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctgggccccccctcgaagtcgacggtattgataagcttgatatcgaattcctgcagcccgggggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 8 - DNA, 11201 bp, synthetic construct
  source 1..11201: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggccctgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggatgaatcaagtattattccggcagagaaagtggccggagctgagaaaaaagagcttcaagggctgcttaagacggcggttcaatctgtggactggacttatagtgtcttctggcaattttgtcctcaacaacgggtcttggtgtgggggaatggatactacaacggtgcaataaagacgaggaagacaactcaaccagcggaggtgacggcggaagaggctgcgttagagaggagccaacagctcagggagctttatgagacacttttagccggagagtcaacgtcagaagcaagagcatgcaccgcattgtcaccggaggatttgacggagacagaatggttttatctaatgtgtgtgtctttctcttttcctcctccatctgggatgccaggaaaagcgtatgcaaggaggaagcacgtatggctaagtggtgcaaatgaagttgacagtaaaactttttctagagctattctcgctaagagtgctaaaattcagacagtggtttgcattccaatgcttgatggtgttgtggaactaggcacaacgaaaaaggtaagagaagatgtagagtttgttgagctcacaaagagtttcttctatgaccactgcaagacgaacccaaagccggctctttctgaacactccacctacgaagtgcatgaagaagccgaagacgaagaagaagtagaagaagagatgacaatgtcagaggaaatgaggcttggctctcctgatgatgaagatgtttccaatcaaaatctacactctgatcttcatattgaatcaacccatacgttagacacacatatggacatgatgaatctaatggaggaaggtggaaactattctcagacagtaacaacacttctcatgtcacaccccacaagtcttctttcagattcagtttccacatattcttacatccaatcatcgtttgccacgtggagggttgagaatggcaaagagcatcagcaagtgaaaacggcgccgtcgtcacaatgggtgctcaaacaaatgatcttcagagttcctttcctccatgacaacactaaagataagaggctaccgcgggaagatctgagccacgtagtagcagagcgacgcaggagggagaagctgaacgagaaattcataacgttgagatcaatggttccatttgtgaccaagatggataaagtctcaatccttggagacaccattgcgtacgtaaatcatcttcgaaagagggtccatgagcttgagaatactcatcatgagcaacagcataagcggacgcgtacttgtaagagaaaaacatcggaggaggtggaggtttccatcatagagaatgatgttttgttagagatgagatgtgagtaccgagatggtttgttgcttgacattcttcaggttcttcatgagcttggtatagagactacggcagttcatacctcggtgaacgaccatgatttcgaggcggagataagggcgaaagtaagagggaagaaagcaagcatcgctgaggtcaaaagagccatccaccaagtcataatacatgatactaatctatagcccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg